(19) United States
(12) Patent Application Publication
(10) Pub. No.: US 2026/0193331 A1
(43) Pub. Date: Jul. 9, 2026

(54) HUMANIZED ANTI-AGR2 ANTIBODY

(71) Applicant: THABOR THERAPEUTICS, Paris (FR)

(72) Inventors: Stéphanie BEQ, Paris (FR); Olivier LEGER, Saint Sixt (FR)

(73) Assignee: THABOR THERAPEUTICS, Paris (FR)

(21) Appl. No.: 19/131,037

(22) PCT Filed: Nov. 21, 2023
(86) PCT No.: PCT/EP2023/082608

§ 371 Date: May 19, 2025

(30) Foreign Application Priority Data
Nov. 21, 2022 (EP) 22306713.3

(51) Int. Cl. C07K 16/18; A61K 39/00; A61P 1/00; G01N 33/68

(52) U.S. Cl. CPC C07K 16/18; A61P 1/00; G01N 33/6893; A61K 2039/505; C07K 2317/24; C07K 2317/33; C07K 2317/34; C07K 2317/92; C07K 2317/94

(57) ABSTRACT

An antibody, or binding fragment thereof, that specifically binds Anterior Gradient 2 protein (AGR2), and the use of the antibody, or binding fragment thereof in treatment methods. Also, a nucleic acid encoding the antibody, or binding fragment thereof, an expression vector including the nucleic acid, a cell including the nucleic acid, and a pharmaceutical composition including the antibody, or binding fragment thereof, the nucleic acid, the expression vector, or the cell.

### FIELD OF INVENTION

The present invention relates to the field of immunotherapy. In particular, the present invention relates to an antibody, or a binding fragment thereof, that specifically binds Anterior Gradient 2 protein (AGR2). The present invention further relates to the use of said antibody, or binding fragment thereof, in therapy.

### BACKGROUND OF INVENTION

Anterior gradient-2 (AGR2) is an endoplasmic reticulum (ER)-resident protein that belongs to the protein disulfide isomerase superfamily. AGR2 shows two localizations: intracellular and extracellular. In healthy cells that express AGR2, the predominant form is the intracellular form, which resides in the endoplasmic reticulum. In contrast, cancer cells express AGR2 on the cell surface and secrete it in the extracellular environment. Thus, AGR2 is not only found in the ER, but also in other locations such as the nucleus, cytoplasm, plasma membrane and extracellular space.

It has been shown that AGR2 exists not only as a monomer, but it can also form homodimers. Indeed, AGR2 forms dimers through residues E60 and C81.

AGR2 protein has a relatively unique property for a chaperone in that it can bind sequence-specifically to a specific peptide motif (TTIYY—SEQ ID NO: 48). Additionally, AGR2 harbors an ER retention signal sequence (KTEL—SEQ ID NO: 49). In normal tissues, AGR proteins could contribute to the regulation of the total protein load in the cell. AGR2 is also involved in pathways for ER stress, protein folding, transcription regulation, and exosome formation. Basal level of AGR2 expression was observed across different tissue types especially for tissues of epithelial origin. In adult human tissues, the highest level of AGR2 expression was observed in the gastrointestinal tract (from the stomach to rectum) and genitourinary tract (urinary bladder and female and male genitalia), as well as in respiratory epithelia of nasopharynx and bronchus (i.e., all mucosal epithelium). In mammals, AGR2 is generally present in mucus secreting epithelial cells and is highly expressed in Paneth and goblet intestinal cells, with the highest levels in the ileum and colon.

AGR2 is a marker of tumor aggressiveness expressed by many solid tumor types. In prostate, AGR2 is overexpressed in cancer cells compared to normal luminal cells, and a majority of primary prostate tumors are AGR2 positive. This pattern is similarly found in pancreatic, oral, and breast cancers. AGR2 is also highly expressed in non-small cell lung cancer where high expression is associated with poor survival.

Studies in mammals revealed the role of AGR2 as a pro-metastatic protein essential to cancer progression and drug resistance. Recent data have highlighted an extracellular role for AGR2 in promoting cancer growth. Cancer-associated function of AGR2 seems to derive from its ability to promote cell adhesion, stimulate cell migration through an extracellular activity, and catalyze plasma membrane receptor trafficking through an intra-cellular function. Recently, specific protein-protein interaction of AGR2 with the oncogenic membrane receptor EpCAM has been shown.

The role of extracellular AGR2 (eAGR2) in the tumor development clearly shows that eAGR2 protein acts as an extracellular regulator, through gain of extracellular functions, on phenotypes associated with tumor morphogenesis, tumorigenicity, and inflammation. The deregulation of AGR2 localizations, intracellular (iAGR2) and extracellular (eAGR2), could exert different pro-oncogenic gain-of-functions.

Due to almost ubiquitous expression in solid tumors, expression in premalignant lesions and its involvement in metastatic disease, AGR2 protein represents a relevant target for cancer therapy.

Besides, AGR2 is also involved in other diseases such as asthma and inflammatory bowel disease. Indeed, studies in transgenic mice have shown that AGR2-null animals are defective in mucin production, have alterations in asthma incidence, and are primed to develop inflammatory bowel disease. Development of these pathologies in AGR2-null animals is due to the important role of intracellular AGR2, which acts as a chaperone in the folding and trafficking of mucin.

Several anti-AGR2 antibodies are commercially available. The epitopes recognized by the commercially available antibodies are not described. These antibodies are mostly sold for western blot or immunohistochemistry applications with no guarantee to function for ELISA or in vitro functional assays. Moreover, they are not described to cross react with other species preventing their use in animal models. Furthermore, these antibodies are poorly referenced in any publications.

The anti-AGR2 clone «1C3» is commercialized by Abnova (H00010551-M03) and was obtained by immunization of mouse using the AGR2 full-length recombinant protein with GST tag, but its epitope is unknown. This monoclonal antibody is a murine IgG2b kappa.

Liu et al. have produced two anti-human AGR2 antibodies, P1G4 and P3A5, intended for use as therapeutic treatment of cancer. The authors demonstrated in vivo an enhancement of Gemcitabine inhibition of tumor growth by P1G4 monoclonal antibody, but not with P3A5.

A humanized anti-AGR2 antibody (18A4Hu) and its murine version (18A4) were reported to have inhibitory effect on AGR2+ ovarian cancer xenograft SK-OV-3.

It should be noted that all these monoclonal antibodies were developed for oncology indications only. When available, data obtained with these antibodies only exhibit inhibitory effect on tumors.

Besides, AGR2 has been shown to be involved in other diseases such as inflammatory diseases.

AGR2 expression is increased in biopsies taken from patients with active ulcerative colitis disease compared to patients in remission and to non IBD controls. AGR2 has also been found to be associated to homeostasis breakdown in pediatric ulcerative colitis disease. Moreover, in ulcerative colitis surgical specimen, AGR2 is highly expressed in colonic epithelium associated to histological fibrosis evidence, while AGR2 immunohistochemical signal is significantly lower in surgical normal margins isolated in non-IBD colons.

Moreover, immuno-histological staining of large-bowel sections from inflammatory bowel disease patients shows that AGR2 expression is highly upregulated compared to non-inflamed controls and localized to all epithelial surfaces (Al-Shaibi et al., Cell Mol Gastroenterol Hepatol, 2021; 12(5):1809-1830).

In inflammatory bowel disease, and specifically in Crohn's disease, the levels of AGR2 dimerization modulators are selectively deregulated, and this correlates with disease severity. AGR2 dimers act as sensors of ER homeostasis, which are disrupted upon ER stress and promote the secretion of AGR2 monomers. The latter might represent systemic alarm signals for pro-inflammatory responses (Maurel et al., EMBO Mol Med, 2019, 11(6):e10120).

AGR2 release in the extracellular environment enhances monocyte recruitment and pro-inflammatory phenotypes. Regulation of AGR2 dimerization is associated with pro-inflammatory responses and enrichment of macrophages in the colonic mucosa that could be observed in Crohn's disease (Maurel et al., EMBO Mol Med, 2019, 11(6):e10120).

The secretion of AGR2 by epithelial cells can participate in the development of fibrosis in Crohn's disease. In the ileum, there is a significant increase of AGR2 in tissues with a fibrotic compartment compared to pure inflammatory samples, highlighting that AGR2 is associated with a fibrostenosis process. AGR2 overexpression at the mRNA level is correlated to fibrosis grade in Crohn's disease patients (Vieujean et al., J Crohns Colitis, 2021, 15(10):1737-1750).

eAGR2, under its monomeric form, has been shown to selectively promote monocyte attraction, thereby linking eAGR2 to pro-inflammatory phenotypes and unraveling the extracellular gain-of-function of AGR2 as a pro-inflammatory chemokine (Maurel et al., EMBO Mol Med, 2019, 11(6):e10120). AGR2 blocking antibodies have been shown to impede monocytes migration and may thus inhibit a very early step of inflammation by blocking local monocyte recruitment.

Moreover, the differentiation of fibroblasts into myofibroblasts obtained when cultured in the presence of supernatant from intestinal epithelial cell pre-conditioned by ER stress, as well as with recombinant AGR2, can be attenuated after blocking AGR2 with an anti-AGR2 antibody. Thus, AGR2 seems to have a pro-fibrotic role and to act as a paracrine inducer of intestinal fibroblast-to-myofibroblast differentiation (Vieujean et al., J Crohns Colitis, 2021, 15(10):1737-1750.

By blocking fibroblast-to-myofibroblast differentiation, anti-AGR2 antibodies may block fibrosis installation preventing stricture in patients.

The etiology of chronic inflammatory bowel diseases such as Crohn's disease or ulcerative colitis is poorly understood. This results in a therapeutic strategy focused on the treatment of inflammatory symptoms without being able to act on the initial cause of the disease.

Less serious cases are treated with amino-salicylate compounds that have a local action on inflammation or corticosteroids. Patients with more severe forms of these diseases or who develop dependence on corticosteroids are treated with so-called biotherapies. The management of inflammatory bowel diseases often involves immunosuppressive treatments such as corticosteroids, immunomodulators, small molecules and biological agents that inhibit pro-inflammatory cytokine pathways (such as, e.g., anti-TNF antibodies).

Anti-TNF (Tumor Necrosis Factor) antibodies are the first-line treatments, but they are effective in only 30 to 40% of patients who go into remission. Moreover, 15% per year of patients responding to anti-TNF develop insensitivity to it, obliging gastroenterologists to use second-line treatments, anti-interleukins (usketinumab, STELARA®, Janssen) and anti-integrins (vedolizumab, ENTYVIO®, Takeda) antibodies.

The efficacy of these biotherapies is good but quickly decreases over time. Above all, the side effects of these treatments, which target the patient's immunity, are numerous: skin disorders, opportunistic infections, risk of cancer, etc. New therapeutic approaches are currently being developed, in particular small molecules targeting the ubiquitous JAK or S1P pathways, without having demonstrated their efficacy or safety for patients.

The use of more traditional immunosuppressants is becoming increasingly rare, given the high associated risks of cancer and serious infections. Surgery remains the most frequent outcome to avoid severe complications (notably colorectal cancer), causing serious alteration of patients' lives.

When patients experience inflammatory flare-ups, remission may be induced with current treatments. However, these treatments must be taken chronically, for life, to avoid relapses. However, a very significant and rapid therapeutic escape is observed in patients, and the serious side effects encourage the greatest caution during prolonged administrations.

Therefore, the discovery of a treatment that can maintain patients in long-term remission, while not presenting risks of side effects, would be a breakthrough in the field of inflammatory diseases of the mucous membranes.

AGR2 is a promising therapeutic target in the context of inflammatory diseases. However, no anti-AGR2 antibody has been developed for and shown to be effective in the prevention or treatment of inflammatory diseases.

Therefore, there is still a need for novel anti-AGR2 antibodies suitable to be used as a therapy, in particular in the context of inflammatory diseases.

Consequently, the inventors herein aimed at developing a novel tool to target AGR2 protein, in particular extracellular AGR2. More specifically, the inventors herein disclose a novel antibody, and binding fragments thereof, that specifically binds AGR2. The antibody, or binding fragment thereof, that specifically binds AGR2 of the present invention would be a valuable therapeutic tool for treating mucosal inflammatory diseases or cancer for example.

By targeting the pathologically abnormal secreted protein (eAGR2), the inventors aim at abrogating a unique mechanism of action: the early crosstalk between the epithelium and the immune system in mucosal inflammatory diseases, while having a much less aggressive secretive profile than competing immunotherapies.

Compared to the standard products currently used in the treatment of mucosal inflammatory diseases, anti-eAGR2 is the only one combining an anti-inflammatory action with an anti-fibrotic action, without risk of side effects, and with a targeted action on the affected tissue.

The inventors have identified several humanized monoclonal antibodies capable of specifically binding to human AGR2 with a high affinity. These humanized antibodies may be used in the prevention or treatment of inflammatory diseases and cancer.

### SUMMARY

The present invention relates to an isolated antibody, or binding fragment thereof, that specifically binds to Anterior Gradient 2 protein (AGR2), wherein said antibody, or binding fragment thereof, comprises:

a heavy chain variable region (VH) comprising
the following three complementary-
determining regions (CDRs):
CDR1:
(SEQ ID NO: 8)
RSWMN;
CDR2:
(SEQ ID NO: 9)
WIYPGDGDTNYNGKXKD
wherein X is F or V;
CDR3:
(SEQ ID NO: 12)
GGYDGSPWLSY;
and
a light chain variable region (VL) comprising
the following three CDRs:
CDR1:
(SEQ ID NO: 13)
KASQDINSYLS;
CDR2:
(SEQ ID NO: 14)
RANRLVD;
and
CDR3:
(SEQ ID NO: 15)
LQYDEFPFT.

In some embodiments the isolated antibody, or binding fragment thereof, that specifically binds to AGR2 comprises:
a heavy chain variable region (VH) comprising framework regions sharing at least 80% sequence identity with the framework regions of SEQ ID NO: 16; and a light chain variable region (VL) comprising framework regions sharing at least 80% sequence identity with the framework regions of SEQ ID NO: 19; or
a heavy chain variable region (VH) comprising framework regions sharing at least 80% sequence identity with the framework regions of SEQ ID NO: 17; and a light chain variable region (VL) comprising framework regions sharing at least 80% sequence identity with the framework regions of SEQ ID NO: 19; or
a heavy chain variable region (VH) comprising framework regions sharing at least 80% sequence identity with the framework regions of SEQ ID NO: 16; and a light chain variable region (VL) comprising framework regions sharing at least 80% sequence identity with framework regions of SEQ ID NO: 20; or
a heavy chain variable region (VH) comprising framework regions sharing at least 80% sequence identity with the framework regions of SEQ ID NO: 17; and a light chain variable region (VL) comprising framework regions sharing at least 80% sequence identity with the framework regions of SEQ ID NO: 20; or
a heavy chain variable region (VH) comprising framework regions sharing at least 80% sequence identity with the framework regions of SEQ ID NO: 18; and a light chain variable region (VL) comprising framework regions sharing at least 80% sequence identity with the framework regions of SEQ ID NO: 21.

In some embodiments, the isolated antibody, or binding fragment thereof, that specifically binds to AGR2 comprises:
a heavy chain variable region (VH) having a sequence consisting of sequence SEQ ID NO: 16; and a light chain variable region (VL) having a sequence consisting of sequence SEQ ID NO: 19; or
a heavy chain variable region (VH) having a sequence consisting of sequence SEQ ID NO: 17; and a light chain variable region (VL) having a sequence consisting of sequence SEQ ID NO: 19; or
a heavy chain variable region (VH) having a sequence consisting of sequence SEQ ID NO: 16; and a light chain variable region (VL) having a sequence consisting of sequence SEQ ID NO: 20; or
a heavy chain variable region (VH) having a sequence consisting of sequence SEQ ID NO: 17; and a light chain variable region (VL) having a sequence consisting of sequence SEQ ID NO: 20; or
a heavy chain variable region (VH) having a sequence consisting of sequence SEQ ID NO: 18; and a light chain variable region (VL) having a sequence consisting of sequence SEQ ID NO: 21.

In some embodiments, the isolated antibody, or binding fragment thereof, that specifically binds to AGR2 is an immunoconjugate.

The present invention also relates to a nucleic acid encoding said antibody, or binding fragment thereof, that specifically binds to AGR2.

The present invention also relates to an expression vector comprising said nucleic acid.

The present invention also relates to a cell comprising said nucleic acid or said expression vector.

The present invention also relates to a pharmaceutical composition comprising said isolated antibody, or binding fragment thereof, that specifically binds to AGR2, said nucleic acid, said expression vector, or said cell, and at least one pharmaceutically acceptable excipient.

The present invention also relates to said isolated antibody, or binding fragment thereof, that specifically binds to AGR2, said nucleic acid, said expression vector, said cell, or said pharmaceutical composition, for use as a medicament.

The present invention also relates to said isolated antibody, or binding fragment thereof, that specifically binds to AGR2, said nucleic acid, said expression vector, said cell, or said pharmaceutical composition, for use in the treatment of a mucosal inflammatory disease, or a cancer, in a subject in need thereof.

In some embodiments, said isolated antibody, or binding fragment thereof, that specifically binds to AGR2, neutralizes the pro-inflammatory activity of eAGR2 and/or the pro-fibrotic activity of eAGR2.

In some embodiments, the mucosal inflammatory disease is selected from the group consisting of Crohn's disease, ulcerative colitis, primary sclerosing cholangitis, chronic pancreatitis, microscopic colitis, inflammatory bowel disease (IBD), endometriosis, appendicitis, inflammatory bowel syndrome, idiopathic pulmonary fibrosis, systemic sclerosis, systemic sclerosis associated with interstitial lung disease, asthma and chronic obstructive pulmonary disease.

In some embodiments, said cancer is selected from the group consisting of colon cancer, gastrointestinal cancer, prostate cancer, pancreatic cancer, oral cancer, breast cancer, lung cancer, ovarian cancer, thyroid cancer, cholangiocarcinoma, head and neck squamous cell carcinoma, brain glioblastoma, adrenocortical carcinoma, bladder cancer, kidney cancer, penile cancer, renal cancer, testicular cancer, urethral cancer, colorectal cancer, cervical cancer, uterine cancer, endometrial cancer, vaginal cancer, vulvar cancer, gestational trophoblastic disease (GTD), and primary peritoneal cancer.

The present invention also relates to an in vitro method for detecting or quantifying AGR2 expression in a biological sample, comprising contacting said biological sample with the isolated antibody, or binding fragment thereof, that specifically binds to AGR2, as described herein.

In some embodiments, said method is for diagnosing or monitoring an AGR2-related disease in a subject, or for selecting a subject suffering from an AGR2-related disease for treatment targeting said disease.

### Definitions

In the present invention, the following terms have the following meanings:

“Affinity” is used to define the strength of an antibody-antigen complex. Affinity measures the strength of interaction between an epitope and an antigen binding site on an antibody. It may be expressed by an affinity constant KA or by a dissociation constant KD.

“Antibody” as used herein, refers to a protein, or polypeptide sequence derived from an immunoglobulin (IgG) molecule which specifically bind s with an antigen. Antibodies can be polyclonal or monoclonal, multiple or single chain, or intact immunoglobulins, and may be derived from natural sources or from recombinant sources. The term “antibody” also includes multispecific antibodies (e.g., bispecific antibodies), and antibody fragments, so long as they exhibit the desired biological activity. Antibodies can be multimers of immunoglobulin molecules, such as tetramers of immunoglobulin molecules. The basic four-chain antibody unit is a heterotetrameric glycoprotein composed of two identical light (L) chains and two identical heavy (H) chains. The L chain from any vertebrate species can be assigned to one of two clearly distinct types, called kappa (κ) and lambda (λ), based on the amino acid sequences of their constant domains (CL). Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha ([α]), delta ([δ]), epsilon ([ε]), gamma ([γ]) and mu ([μ]), respectively. The [γ] and [α] classes are further divided into subclasses based on relatively minor differences in CH sequence and function, e.g., humans express the following subclasses: IgG1, IgG2, IgG3, IgG4, IgA1, and IgA2. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the [α] and [γ] chains and four CH domains for [μ] and [ε] isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and a VL together forms a single antigen-binding site. An IgM antibody consists of five of the basic heterotetramer units along with an additional polypeptide called a J chain, and therefore, contains ten antigen-binding sites, while secreted IgA antibodies can polymerize to form polyvalent assemblages comprising 2-5 of the basic 4-chain units along with J chain. In the case of IgGs, the 4-chain unit is generally about 150,000 Daltons.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, a v-NAR and a bis-scFv. Antigen binding fragments can also be grafted into scaffolds based on polypeptides such as a fibronectin type III. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of crosslinking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

As used herein, the term “binding fragment”, refers to a part or region of the antibody according to the present invention, which comprises fewer amino acid residues than the whole antibody. A “binding fragment” binds antigen and/or competes with the whole antibody from which it was derived for antigen binding. Antibody binding fragments encompasses, without any limitation, single chain antibodies, Fv, Fab, Fab′, Fab′-SH, F(ab)′2, Fd, defucosylated antibodies, diabodies, triabodies and tetrabodies.

“Antigen” or “Ag” refers to a molecule that provokes an immune response. This immune response may involve either antibody production, and/or the activation of specific immunologically-competent cells, or both.

“Cancer” generally refers to a disease caused by an uncontrolled division of abnormal cells. The term “cancer” in particular refers to any disease associated with tumorigenesis. The term “cancer” encompasses solid tumors and blood cancers, and encompasses both primary and metastatic cancers.

“CDR” or “complementarity determining region” means the non-contiguous antigen combining sites found within the variable region of both heavy and light chain polypeptides. The precise amino acid sequence boundaries of a given CDR can be determined using any of several well-known schemes, including those described by Kabat et al. (1991), “Sequences of Proteins of Immunological Interest” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof. More recently, a universal numbering system has been developed and widely adopted, ImMunoGeneTics (IMGT) Information System® (Lefranc et al., Nucleic Acids Res. 27:209-212 1999). Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues may be readily identified. This information can be used in grafting and replacement of CDR residues from immunoglobulins of one species into an acceptor framework from, typically, a human antibody. In some embodiments, by CDR regions or CDR, it is intended to indicate the hypervariable regions of the heavy and light chains of the immunoglobulins as defined by Kabat et al. (1991), (“Kabat” numbering scheme).

“Epitope” refers to a specific arrangement of amino acids located on a protein or proteins to which an antibody binds. Epitopes often consist of a chemically active surface grouping of molecules such as amino acids or sugar side chains and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Framework region” or “FR region” includes the amino acid residues of an antibody, or binding fragment thereof, that are part of the variable region but are not part of the CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions.

“Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., a, 8, ¿ and u for human antibodies), or a naturally occurring allotype thereof.

“Heavy chain region” includes amino acid sequences derived from the constant domains of an immunoglobulin heavy chain. A protein comprising a heavy chain region comprises at least one of a CH1 domain, a hinge (e.g., upper, middle, and/or lower hinge region) domain, a CH2 domain, a CH3 domain, or a variant or fragment thereof. In an embodiment, the antibody according to the present invention may comprise the Fc region of an immunoglobulin heavy chain (e.g., a hinge portion, a CH2 domain, and a CH3 domain). In some embodiments, the antibody according to the present invention may comprise a heavy chain region comprising all of the constant domains derived from a human immunoglobulin 1 (IgG1) heavy chain. In some embodiments, the constant domains of the heavy chain region may be modified such that they vary in amino acid sequence from the naturally occurring (wild-type) immunoglobulin molecule. That is, the antibody according to the present invention may comprise alterations or modifications to one or more of the heavy chain constant domains (C_{H}1, hinge, C_{H}2 or C_{H}3) and/or to the light chain constant domain (CL). Exemplary modifications include additions, deletions or substitutions of one or more amino acids in one or more domains.

Within an antibody, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains.

“Identity” or “identical”, when used herein in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

As used herein, the term “immune cells” generally includes white blood cells (leukocytes) that are derived from hematopoietic stem cells (HSC) produced in the bone marrow. Examples of immune cells include, but are not limited to, lymphocytes (T cells, B cells, and natural killer (NK) cells) and myeloid-derived cells (neutrophil, eosinophil, basophil, monocyte, macrophage, dendritic cells).

As used herein, the term “isolated” or “non-naturally occurring” with reference to a biological component (such as a nucleic acid molecule, a protein or a cell), refers to a biological component altered or removed from the natural state. For example, a nucleic acid or a peptide naturally present in a living animal is not “isolated” but the same nucleic acid or peptide partially or completely separated from the coexisting materials of its natural state is “isolated”. An isolated nucleic acid or peptide can exist in substantially purified form, or can exist in a non-native environment such as, for example, a host cell. Typically, a preparation of isolated nucleic acid or peptide contains the nucleic acid or peptide at least about 80% pure, at least about 85% pure, at least about 90% pure, at least about 95% pure, greater than 95% pure, greater than about 96% pure, greater than about 97% pure, greater than about 98% pure, or greater than about 99% pure. Nucleic acids and proteins that are “non-naturally occurring” or have been “isolated” include nucleic acids and proteins purified by standard purification methods. The term also embraces nucleic acids and proteins prepared by recombinant expression in a host cell as well as chemically synthesized nucleic acids. An “isolated polypeptide” is one that has been identified and separated and/or recovered from a component of its natural environment.

As used herein, the term “monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

As used herein, the term “nucleic acid” or “polynucleotide” refers to a polymer of nucleotides covalently linked by phosphodiester bonds, such as deoxyribonucleic acids (DNA) or ribonucleic acids (RNA), in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues.

As used herein, the terms “prevent”, “preventing” and “prevention” refer to prophylactic and preventative measures, wherein the object is to reduce the chances that a subject develop the pathologic condition or disorder over a given period of time. Such a reduction may be reflected, e.g., in a delayed onset of at least one symptom of the pathologic condition or disorder in the subject.

“Single-chain variable fragment”, also abbreviated as “sFv” or “scFv”, refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. Divalent, trivalent and higher scFv, called tandem-di-scFv, tandem-tri-scFv, etc., can be engineered by linking two, three or more scFv together. Alternatively, two scFv can be forced to dimerize into a diabody by using a linker peptide that is too short (typically of about 5 to 10 amino acids) between the V_{H} and the V_{L} domains. Alternatively, an even shorter linker peptide (typically of about 1 to 2 amino acids) leads to the formation of scFv trimers, also called triabodies or tribodies.

“Subject” is intended to include living organisms in which an immune response can be elicited. Preferably, the term “subject” refers to a warm-blooded animal, more preferably a mammal. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human. In some embodiments, a subject may be a “patient”, who/which is awaiting the receipt of, or is receiving medical care or was/is/will be the object of a medical procedure, or is monitored for the development of the targeted disease or condition, such as, for example, mucosal inflammatory diseases. In some embodiments, the subject is an adult (for example a subject above the age of 18). In some embodiments, the subject is a child (for example a subject below the age of 18). In some embodiments, the subject is a male. In some embodiments, the subject is a female. In some embodiments, the subject is affected, preferably is diagnosed, with a mucosal inflammatory disease. In some embodiments, the subject is at risk of developing a mucosal inflammatory disease. Examples of risks factor include, but are not limited to, genetic predisposition, or familial history of mucosal inflammatory diseases.

As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

“Therapeutically effective amount” refers to the level or amount of an antibody, or binding fragment thereof, as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively, or additionally, the therapeutically effective amount may be administered after initiation of the disease, disorder, or condition, for a therapeutic action.

“Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. In some embodiments, a subject is successfully “treated” for a disease or disorder if, after receiving a therapeutic amount of an antibody, or binding fragment thereof, according to the present invention, the subject shows at least one of the following: relief to some extent of one or more of the symptoms associated with the disease or disorder to be treated; reduced morbidity and mortality; and improvement in quality-of-life issues. The above parameters for assessing successful treatment and improvement in the disease are readily measurable by routine procedures familiar to a physician.

As used herein, the terms “variable”, “variable region” or “variable domain” refer to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “complementarity determining region” or “CDR” in each of the V_{L} domain and the V_{H} domain which form part of the antigen binding site.

### DETAILED DESCRIPTION

Human AGR2 typically refers to the protein referenced as AAY84776.1 in the NCBI databases on Jan. 19, 2007. In the NCBI databases (https://www.ncbi.nlm.nih.gov), the reference human AGR2 gene sequence corresponds to NCBI Gene ID: 10551, as updated on Nov. 28, 2021. The human AGR2 gene consists of 8 exons on chromosome 7p21.1. AGR2 transcript encompasses 1697 nucleotides and encodes a 175 amino acid protein. The reference human AGR2 protein sequence corresponds to SEQ ID NO: 1.

Mouse AGR2 typically refers to the protein referenced as NP_035913.1 in the NCBI databases on Dec. 4, 2021. In the NCBI databases, the reference mouse AGR2 gene sequence corresponds to NCBI Gene ID: 23795, as updated on Nov. 30, 2021. The mouse AGR2 gene consists of 8 exons on chromosome 12; 12A. AGR2 transcript encompasses 760 nucleotides and encodes a 175 amino acid protein. The reference mouse AGR2 protein sequence corresponds to SEQ ID NO: 2.

Rhesus monkey AGR2 typically refers to the protein referenced as NP_001181233.1 in the NCBI databases Jul. 11, 2020. In the NCBI databases, the reference rhesus monkey AGR2 gene sequence corresponds to NCBI Gene ID: 709127, as updated on Jun. 24, 2020. The rhesus monkey AGR2 gene consists of 8 exons on chromosome 3. AGR2 transcript encompasses 1808 nucleotides and encodes a 175 amino acid protein. The reference rhesus monkey AGR2 protein sequence corresponds to SEQ ID NO: 3.

Chimpanzee AGR2 typically refers to the protein referenced as XP_003318381.1 in the NCBI databases Mar. 20, 2018. In the NCBI databases, the reference chimpanzee AGR2 gene sequence corresponds to NCBI Gene ID: 463277, as updated on Mar. 19, 2021. The chimpanzee AGR2 gene consists of 8 exons on chromosome 7. AGR2 transcript encompasses 2197 nucleotides and encodes a 175 amino acid protein. The reference chimpanzee AGR2 protein sequence corresponds to SEQ ID NO: 4.

Rat AGR2 typically refers to the protein referenced as NP_001100195.1 in the NCBI databases Feb. 1, 2021. In the NCBI databases, the reference rat AGR2 gene sequence corresponds to NCBI Gene ID: 298961, as updated on Dec. 15, 2021. The rat AGR2 gene consists of 9 exons on chromosome 6q16. AGR2 transcript encompasses 961 nucleotides and encodes a 175 amino acid protein. The reference rat AGR2 protein sequence corresponds to SEQ ID NO: 5.

Dog AGR2 typically refers to the protein referenced as XP_038542570.1 in the NCBI database on Jan. 7, 2021. In the NCBI databases, the reference dog AGR2 gene sequence corresponds to NCBI Gene ID: 482333, as updated on Dec. 15, 2021. The dog AGR2 gene consists of 9 exons on chromosome 14. AGR2 transcript encompasses 2657 nucleotides and encodes a 175 amino acid protein. The reference dog AGR2 protein sequence corresponds to SEQ ID NO: 6.

Alternatives names for AGR2 include “Anterior Gradient 2 Protein”, “AG-2”, “AG2”, “HPC8”, “GOB-4”, “HAG-2”, “XAG-2”, “PADIA17”, “HEL-S-116”, “Protein Disulfide Isomerase Family A Member 17”, and “Secreted cement gland protein XAG-2 homolog” as non-limiting examples. Herein, the expressions “Anterior Gradient 2 Protein” and “AGR2” and “AG-2” are used indifferently.

In the context of the invention, AGR2 refers to the human AGR2 as well as any homologous protein in any animal species. For instance, AGR2 refers to any homologous sequence corresponding to SEQ ID NO: 1, SEQ ID NO: 2, SEQ ID NO: 3, SEQ ID NO: 4, SEQ ID NO: 5 or SEQ ID NO: 6 in any other species.

AGR2 protein has an N-terminal signal peptide sequence, a catalytically active thioredoxin domain, and a C-terminal ER-retention sequence. AGR2 plays a role in cell migration, cellular transformation and metastasis and is as a p53 inhibitor. As an ER-localized molecular chaperone, it plays a role in the folding, trafficking, and assembly of cysteine-rich transmembrane receptors and the cysteine-rich intestinal glycoprotein mucin. Extracellular (or secreted) AGR2 also displays proinflammatory and profibrotic activities.

AGR2 protein can be found in different localizations: it may be intracellular or extracellular, either bound to the cell surface or in a circulating (secreted) form.

The present invention firstly relates to an isolated antibody, or to a binding fragment thereof, that specifically binds to Anterior Gradient 2 protein (AGR2).

An “isolated antibody”, as used herein, is intended to refer to an antibody that is modified or removed from its natural state. In particular, an isolated antibody may be substantially free of other antibodies having different antigenic specificities (e.g., an isolated antibody that specifically binds AGR2 is substantially free of antibodies that specifically bind antigens other than AGR2). An isolated antibody that specifically binds AGR2 may, however, have cross-reactivity to other antigens, such as AGR2 molecules from other species. Moreover, an isolated antibody may be substantially free of other cellular material and/or chemicals, in particular those that would interfere with therapeutic uses of the antibody, including without limitation, enzymes, hormones, and other proteinaceous or non-proteinaceous components.

Preferably, the isolated antibody, or binding fragment thereof, is purified. For instance, the isolated antibody, or binding fragment thereof, is purified to greater than 80%, 85%, 90%, 91%, 92%, 93%, 94%, 95% or more by weight of protein.

The isolated antibody, or binding fragment thereof, of the invention specifically binds to AGR2.

The isolated antibody, or binding fragment thereof, of the invention specifically binds to all forms of AGR2, in particular to intracellular AGR2, extracellular AGR2 (i.e., expressed on the cell surface) and circulating AGR2 (i.e., secreted in the extracellular environment).

In particular, the isolated antibody, or binding fragment thereof, of the invention does not specifically bind to AGR3.

The human AGR2 and AGR3 genes map to chromosome band 7p21.3. The AGR2 and AGR3 proteins are clustered together by phylogenetic analysis and share 65% sequence identity. AGR3 is the closest family member to AGR2.As used herein, an antibody, or binding fragment thereof, is said to be “specific for”, “immunospecific for”, or to “specifically bind” an antigen if it reacts at a detectable level with said antigen (e.g., AGR2), preferably with an affinity constant (K_{A}) greater than or equal to about 10⁶ M⁻¹, preferably greater than or equal to about 107 M⁻¹, 10⁸ M⁻¹, 5×10⁸ M⁻¹, 10⁹ M⁻¹, 5×10⁹ M⁻¹ or more. Affinity of an antibody, or binding fragment thereof, for its cognate antigen is also commonly expressed as an equilibrium dissociation constant (K_{D}). An antibody, or binding fragment thereof, is said to be “specific for” or to “specifically bind” an antigen if it reacts at a detectable level with said antigen (e.g., AGR2), preferably with a K_{D} of less than or equal to 10⁻⁶ M, preferably less than or equal to 10⁻⁷ M, 5×10⁻⁸ M, 10⁻⁸ M, 5×10⁻⁹ M, 10⁻⁹ M or less. Affinities of antibodies, or binding fragment thereof, can be readily determined using conventional techniques, for example, those described by Scatchard, 1949. Ann NY Acad Sci. 51:660-672. Binding properties of an antibody, or binding fragment thereof, to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®).

In some embodiments, the isolated antibody, or binding fragment thereof, that specifically binds to AGR2 of the invention recognizes and is capable to bind to a soluble (i.e., not membrane bound) AGR2 protein. In some embodiments, the isolated antibody, or binding fragment thereof, that specifically binds to AGR2 of the invention recognizes and is capable to bind to a membrane-bound AGR2 protein.

In some embodiments, the isolated antibody, or binding fragment thereof, that specifically binds to AGR2 of the invention recognizes and binds to monomeric AGR2. In some embodiments, the isolated antibody, or binding fragment thereof, of the invention recognizes and binds to human AGR2 of SEQ ID NO: 1 comprising a mutation consisting of the glutamic acid residue at position 60 being substituted by an alanine residue. In some embodiments the isolated antibody, or binding fragment thereof, that specifically binds to AGR2 of the invention recognizes and binds to dimeric AGR2. In some embodiments, the isolated antibody, or binding fragment thereof, that specifically binds to AGR2 of the invention recognizes and binds both monomeric and dimeric forms of the human AGR2 protein with the same affinity.

The isolated antibody, or binding fragment thereof, of the invention recognizes and binds to a human AGR2 protein. Preferably, the isolated antibody, or binding fragment thereof, that specifically binds to AGR2 also binds to one or more homologous AGR2 protein(s) from another species. For instance, the isolated antibody, or binding fragment thereof, that specifically binds to AGR2 may also bind to one or more AGR2 protein(s) selected from a mouse AGR2 protein, a rhesus monkey AGR2 protein, a chimpanzee AGR2 protein, a rat AGR2 protein or a dog AGR2 protein.

In some embodiments, the isolated antibody, or binding fragment thereof, that specifically binds to AGR2 of the invention recognizes and binds to a mouse AGR2 protein. In some embodiments, the isolated antibody, or binding fragment thereof, that specifically binds to AGR2 of the invention recognizes and binds to a rhesus monkey AGR2 protein. In some embodiments, the isolated antibody, or binding fragment thereof, that specifically binds to AGR2 of the invention recognizes and binds to a chimpanzee AGR2 protein. In some embodiments, the isolated antibody, or binding fragment thereof, that specifically binds to AGR2 of the invention recognizes and binds to a rat AGR2 protein. In some embodiments, the isolated antibody, or binding fragment thereof, that specifically binds to AGR2 of the invention recognizes and binds to a dog AGR2 protein.

The isolated antibody of the invention, or binding fragment thereof, recognizes and binds to the human AGR2 protein of sequence SEQ ID NO: 1. Preferably, the isolated antibody, or binding fragment thereof, that specifically binds to AGR2 also binds to one or more AGR2 protein(s) of sequence SEQ ID NO: 2, SEQ ID NO: 3, SEQ ID NO: 4, SEQ ID NO: 5 and/or SEQ ID NO: 6.

In some embodiments, the isolated antibody, or binding fragment thereof, that specifically binds to AGR2 of the invention recognizes and binds to a mouse AGR2 protein of SEQ ID NO: 2. In some embodiments, the isolated antibody, or binding fragment thereof, that specifically binds to AGR2 of the invention recognizes and binds to a rhesus monkey AGR2 protein of SEQ ID NO: 3. In some embodiments, the isolated antibody, or binding fragment thereof, that specifically binds to AGR2 of the invention recognizes and binds to a chimpanzee AGR2 protein of SEQ ID NO: 4. In some embodiments, the isolated antibody, or binding fragment thereof, that specifically binds to AGR2 of the invention recognizes and binds to a rat AGR2 protein of SEQ ID NO: 5. In some embodiments, the isolated antibody, or binding fragment thereof, that specifically binds to AGR2 of the invention recognizes and binds to a dog AGR2 protein of SEQ ID NO: 6. In some embodiments, the isolated antibody, or binding fragment thereof, that specifically binds to AGR2 of the invention recognizes and binds to a monomeric AGR2 protein of SEQ ID NO: 7.

In some embodiments, the isolated antibody, or binding fragment thereof, that specifically binds to AGR2 of the invention recognizes and is capable to bind to an AGR2 variant, preferably a variant of a human AGR2 protein, a variant of a mouse AGR2 protein, a variant of a rhesus monkey AGR2 protein, a variant of a chimpanzee AGR2 protein, a variant of a rat AGR2 protein, and/or a variant of a dog AGR2 protein.

A “variant” or “derivative” protein is defined as having a sequence identical to at least 80%, preferably at least 85%, more preferably at least 90%, even at least 95%, 96%, 97%, 98% or 99% of the reference sequence.

These variant sequences may differ from the reference sequence by substitution, deletion and/or insertion of one or more amino acids. The substitutions may in particular correspond to conservative substitutions or to substitutions of natural amino acids by non-natural amino acids or pseudo amino acids.

By “amino acid sequence having (for instance) at least 80% of identity with a reference sequence” is meant herein a sequence identical to the reference sequence but this sequence may comprise up to twenty mutations (substitutions, deletions and/or insertions) per each part of one hundred amino acids of the reference sequence. Therefore, for a reference sequence of 100 amino acids, a fragment of 80 amino acids and a sequence of 100 amino acids comprising 20 substitutions compared with the reference sequence are two examples of sequences having 80% sequence identity with the reference sequence.

Percentage of identity is generally determined using sequence analysis software (for example the Sequence Analysis Software Package of the Genetics Computer Group, University of Wisconsin Biotechnology Center, 1710 University Avenue, Madison, Wis. 53705). The amino acid sequences to be compared are aligned to obtain maximum percentage identity. For this purpose, it may be necessary to artificially add gaps in the sequence. The alignment can be performed manually or automatically. Automated alignment algorithms of nucleotide sequences are well known to persons skilled in the art and described for example in Altschul et al. (1997) Nucleic Acids Res. 25:3389 and implemented by softwares such as the Blast software. One algorithm which can be isolated is the Needleman-Wunsch algorithm for example (Needleman and Wunsch (1970) J Mol Biol. 48:443-53). Once optimal alignment has been achieved, the percentage identity is established by recording all the positions at which the amino acids of the two compared sequences are identical, compared with the total number of positions.

In particular embodiments, the sequence of the AGR2 protein differs from the reference sequence solely through the presence of conservative substitutions. Conservative substitutions are substitutions of amino acids of the same class, such as substitutions of amino acids with non-charged side chains (such as asparagine, glutamine, serine, cysteine, and tyrosine), of amino acids with basic side chains (such as lysine, arginine and histidine), of amino acids with acid side chains (such as aspartic acid and glutamic acid), of amino acids with non-polar side chains (such as alanine, valine, leucine, isoleucine, proline, phenylalanine, methionine and tryptophan).

Preferably, a variant of AGR2 refers to an AGR2 protein having a sequence wherein at most 1, 2, 3, 4, 5, 6, 7, 8, 9, 10, 15, 20, 25 or 35 amino acids are deleted, added, or substituted as compared to the original protein sequence. In some embodiments, a variant protein of AGR2 refers to an AGR2 protein having a sequence wherein at most 1, 2, 3, 4, 5, 6, 7, 8, 9, 10, 15, 20, 25 or 35 amino acids are deleted, added or substituted as compared to the sequence of SEQ ID NO: 1, SEQ ID NO: 2, SEQ ID NO: 3, SEQ ID NO: 4, SEQ ID NO: 5 or SEQ ID NO: 6.

According to the invention, the polypeptides may be modified chemically or enzymatically to improve their stability or bioavailability. Such chemical or enzymatic modifications are well known to those skilled in the art. Mention may be made of the following modifications, but they are not limited thereto:
modifications of the C-terminal or N-terminal end of the polypeptides such as N-terminal deamination or acylation (preferably acetylation) or such as C-terminal amidation or esterification;
modifications of the amide bond between two amino acids, such as acylation (preferably acetylation) or alkylation at the nitrogen or alpha carbon;
changes in chirality, such as the substitution of a natural amino acid (L-enantiomer) by the corresponding D-enantiomer; this modification may optionally be accompanied by inversion of the side chain (from the C-terminal end to the N-terminal end);
changes to azapeptides, in which one or more alpha carbons are replaced by nitrogen atoms; and/or
changes to betapeptides, in which one or more carbons are added on the N-alpha side or on the C-alpha side of the main chain.

In this respect, it is possible to modify one or more of the lysine amino acids (K) of the polypeptides, notably by:
amidation: this modification is simple to achieve, the positive charge of the lysine being substituted by hydrophobic groups (for example acetyl or phenylacetyl);
amination: by formation of secondary amide from the primary amine R═(CH₂)₄—NH₃⁺, for example by forming N-methyl, N-allyl or N-benzyl groups; and
by formation of N-oxide, N-nitroso, N-dialkyl phosphoryl, N-sulfenyl, or N-glycoside groups.

It is also or alternatively possible to modify one or more threonine (T) and/or serine(S) amino acids of the polypeptides, notably by adding an ester or ether group at the OH group of the side chain of threonine and/or serine. Esterification, a simple operation, can be performed using a carboxylic acid, an anhydride, by bridging, etc., to form acetates or benzoates. Etherification, which gives more stable compounds, can be performed using an alcohol, a halide, etc. to form a methyl ether for example or an O-glycoside.

It is also or alternatively possible to modify one or more glutamine (Q) amino acids for example by amidation, by forming secondary or tertiary amines, in particular with groups of methyl, ethyl type, whether or not functionalized.

It is also or alternatively possible to modify one or more glutamate (E) and/or aspartate (D) amino acids, for example:
by esterification, to form methyl esters, whether or not substituted, ethyl esters, benzyl esters, thiols (activated esters); and
by amidation, notably to form N,N dimethyl groups, nitroanilides, pyrrolidinyls.

In some embodiments, the isolated antibody, or binding fragment thereof, of the invention comprises chemical modifications of one or more:
methionine (M) and/or tryptophan (W) by oxidation,
asparagine (N) by deamidation, and/or
aspartic acid (D) by isomerization.

On the other hand, it is preferable not to modify the proline amino acids, which take part in the secondary structure of the polypeptides.

In some embodiments, the isolated antibody, or binding fragment thereof, that specifically binds to AGR2 of the invention recognizes and is capable to bind to an AGR2 fragment, preferably a fragment of a human AGR2 protein, a fragment of a mouse AGR2 protein, a fragment of a rhesus monkey AGR2 protein, a fragment of a chimpanzee AGR2 protein, a fragment of a rat AGR2 protein, and/or a fragment of a dog AGR2 protein.

As used herein, the term “fragment” of an antigen refers to any subset of an antigen, as a shorter peptide. In some embodiments, a fragment of an antigen is a peptide of at least 6 amino acids in length. In some embodiments, a fragment of an antigen is a peptide of 6 to 50 amino acids in length, of 6 to 30 amino acids, or of 6 to 20 amino acids in length.

The isolated antibody, or binding fragment thereof, that specifically binds to AGR2 of the invention may be polyclonal or monoclonal.

Preferably, the isolated antibody, or binding fragment thereof, that specifically binds to AGR2 is monoclonal.

The isolated antibody that specifically binds to AGR2 of the invention may be a whole antibody or a binding fragment of an antibody. Preferably, the isolated antibody that specifically binds to AGR2 is a whole antibody.

The antigen-binding fragment that specifically binds to AGR2 of the invention may be a molecule selected from the group comprising or consisting of a single-chain antibody, a dimeric single chain antibody, a single-domain antibody, a Fv, a Fab, a Fab′, a Fab′-SH, a F(ab)′2, a Fd, a defucosylated antibody, a bi-specific antibody, a diabody, a triabody and a tetrabody.

The antigen-binding fragment that specifically binds to AGR2 of the invention may be a single-chain antibody. In some embodiments, the single-chain antibody is selected from the group comprising or consisting of a single-chain variable fragment (scFv), a tandem-di-scFv, a tandem-tri-scFv, a scFv-Fc, a (scFv-CH3) 2 (also termed minibody), a (scFv-CH2-CH3)2 (also termed maxibody), a diabody, and a triabody.

The term “binding fragment”, as used herein, refers to a part or region of the antibody according to the present invention, which comprises fewer amino acid residues than the whole antibody. A “binding fragment” binds antigen and/or competes with the whole antibody from which it was derived for antigen binding (e.g., specific binding to AGR2). Antibody binding fragments encompasses, without any limitation, single chain antibodies, Fv, Fab, Fab′, Fab′-SH, F(ab)′2, Fd, defucosylated antibodies, diabodies, triabodies and tetrabodies.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding.

“Fv”, as used herein, refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Diabodies”, as used herein, refers to small antibody fragments prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the HCVR and LCVR of the two antibodies are present on different polypeptide chains.

Antibody binding fragments can be obtained using standard methods. For instance, Fab or F(ab′)2 fragments may be produced by protease digestion of the isolated antibodies, according to conventional techniques.

In some embodiments, the antibody or binding fragment thereof according to the present invention is a molecule selected from the group comprising or consisting of a unibody, a domain antibody, and a nanobody.

“Unibodies” refer to antibody fragments lacking the hinge region of IgG4 antibodies. The deletion of the hinge region results in a molecule that is essentially half the size of traditional IgG4 antibodies and has a univalent binding region rather than the bivalent biding region of IgG4 antibodies.

“Domain antibodies” refer to the smallest functional binding units of antibodies, corresponding to the variable regions of either the heavy or light chains of antibodies.

“Single-domain antibodies” refer to antibody-derived proteins that contain the unique structural and functional properties of naturally-occurring heavy chain antibodies. These heavy chain antibodies may contain a single variable domain (V_{H}H)-one such example is Nanobodies®—, or a single variable domain (V_{H}H) and two constant domains (C_{H}2 and C_{H}3)—such as camelid antibodies—, or a single variable domain (V_{H}H) and five constant domains (C_{H}1, C_{H}2, C_{H}3, C_{H}4 and C_{H}5)—such as shark antibodies.

In one embodiment, the antibody or binding fragment thereof according to the present invention is a mimetic selected from the group comprising or consisting of an affibody, an affilin, an affitin, an adnectin, an atrimer, an evasin, a DARPin, an anticalin, an avimer, a fynomer, a versabody and a duocalin.

“Affibodies” refer to affinity proteins based on a 58 amino acid residue protein domain, derived from one of the IgG binding domain of staphylococcal protein A.

“DARPins” (Designed Ankyrin Repeat Proteins) refer to an antibody mimetic DRP (designed repeat protein) technology developed to exploit the binding abilities of non-antibody proteins.

“Anticalins” refer to another antibody mimetic technology, wherein the binding specificity is derived from lipocalins. Anticalins may also be formatted as dual targeting protein, called “duocalins”.

“Avimers” refer to another antibody mimetic technology.

“Versabodies” refer to another antibody mimetic technology. They are small proteins of 3-5 kDa with >15% cysteines, which form a high disulfide density scaffold, replacing the hydrophobic core the typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T cell epitopes, because the residues that contribute most to MHC presentation are hydrophobic. All four of these properties are well-known to affect immunogenicity, and together they are expected to cause a large decrease in immunogenicity.

In some embodiments, the antibody or binding fragment thereof according to the present invention also encompasses multispecific antibodies or binding fragments thereof, i.e., being immunospecific for more than one, such as at least two, different antigens, one of which being AGR2 according to the present invention.

In some embodiments, the antibody or binding fragment thereof according to the present invention also encompasses polymers of antibodies or binding fragments thereof, i.e., more than one, such as at least two, antibodies or binding fragments thereof, whether identical or different, being covalently linked together, directly or indirectly.

The isolated antibody, or binding fragment thereof, that specifically binds to AGR2 of the invention is humanized.

The complementary-determining regions (CDRs) are herein determined using the Kabat numbering system.

The isolated antibody, or binding fragment thereof, that specifically binds to AGR2 of the present invention comprises:

a heavy chain variable region
(abbreviated herein as VH) comprising the
following three CDRs:
VH CDR1:
(SEQ ID NO: 8)
RSWMN;
VH CDR2:
(SEQ ID NO: 9)
WIYPGDGDTNYNGKXKD,
wherein
X is For V,
VH CDR3:
(SEQ ID NO: 12)
GGYDGSPWLSY;
and
a light chain variable region (abbreviated
herein as VL) comprising the following
three CDRs:
VL CDR1:
(SEQ ID NO: 13)
KASQDINSYLS;
VL CDR2:
(SEQ ID NO: 14)
RANRLVD;
VL CDR3:
(SEQ ID NO: 15)
LQYDEFPFT.

Preferably, the isolated antibody, or binding fragment thereof, that specifically binds to AGR2 comprises:

a heavy chain variable region (abbreviated
herein as VH) comprising the
following three CDRs:
VH CDR1:
(SEQ ID NO: 8)
RSWMN;
VH CDR2:
(SEQ ID NO: 10)
WIYPGDGDTNYNGKFKD
or
(SEQ ID NO: 11)
WIYPGDGDTNYNGKVKD;
and
VH CDR3:
(SEQ ID NO: 12)
GGYDGSPWLSY;

or a set of 3 CDRs having amino acid sequences at least 85%, 90% or 95% identical to SEQ ID NO: 8, 10 or 11 and 12; and

a light chain variable region (abbreviated
herein as VL) comprising the following
three CDRs:
VL CDR1:
(SEQ ID NO: 13)
KASQDINSYLS;
VL CDR2:
(SEQ ID NO: 14)
RANRLVD;
VL CDR3
(SEQ ID NO: 15)
LQYDEFPFT;

or a set of 3 CDRs having amino acid sequences at least 85%, 90% or 95% identical to SEQ ID NO: 13, 14 and 15.

By “a set of 3 CDRs having amino acid sequences at least 85%, 90% or 95% identical to SEQ ID NO:” it is meant a set of 3 CDRs wherein:
VH CDR1 as an amino acid sequence at least 85%, 90% or 95% identical to SEQ ID NO: 8;
VH CDR2 as an amino acid sequence at least 85%, 90% or 95% identical to SEQ ID NO: 10 or 11; and
VH CDR3 as an amino acid sequence at least 85%, 90% or 95% identical to SEQ ID NO: 12.

By “a set of 3 CDRs having amino acid sequences at least 85%, 90% or 95% identical to SEQ ID NO:” it is meant a set of 3 CDRs wherein:
VL CDR1 as an amino acid sequence at least 85%, 90% or 95% identical to SEQ ID NO: 13;
VL CDR2 as an amino acid sequence at least 85%, 90% or 95% identical to SEQ ID NO: 14; and
VL CDR3 as an amino acid sequence at least 85%, 90% or 95% identical to SEQ ID NO: 15.

Preferably, the isolated antibody, or binding fragment thereof, that specifically binds to AGR2 comprises:

a heavy chain variable region (abbreviated
herein as VH) comprising the
following three CDRs:
VH CDR1:
(SEQ ID NO: 8)
RSWMN;
VH CDR2:
(SEQ ID NO: 10)
WIYPGDGDTNYNGKFKD
or
(SEQ ID NO: 11)
WIYPGDGDTNYNGKVKD;
VH CDR3:
(SEQ ID NO: 12)
GGYDGSPWLSY;
and
a light chain variable region (abbreviated
herein as VL) comprising the following
three CDRs:
VL CDR1:
(SEQ ID NO: 13)
KASQDINSYLS;
VL CDR2:
(SEQ ID NO: 14)
RANRLVD;
VL CDR3:
(SEQ ID NO: 15)
LQYDEFPFT.

Preferably, the isolated antibody, or binding fragment thereof, that specifically binds to AGR2 comprises:

a heavy chain variable region (abbreviated
herein as VH) comprising the
following three CDRs:
VH CDR1:
(SEQ ID NO: 8)
RSWMN;
VH CDR2:
(SEQ ID NO: 10)
WIYPGDGDTNYNGKFKD;
VH CDR3:
(SEQ ID NO: 12)
GGYDGSPWLSY;
and
a light chain variable region (abbreviated
herein as VL) comprising the following
three CDRs:
VL CDR1:
(SEQ ID NO: 13)
KASQDINSYLS;
VL CDR2:
(SEQ ID NO: 14)
RANRLVD;
VL CDR3:
(SEQ ID NO: 15)
LQYDEFPFT.

Preferably, the isolated antibody, or binding fragment thereof, that specifically binds to AGR2 comprises:

a heavy chain variable region (abbreviated
herein as VH) comprising the
following three CDRs:
VH CDR1:
(SEQ ID NO: 8)
RSWMN;
VH CDR2:
(SEQ ID NO: 11)
WIYPGDGDTNYNGKVKD;
VH CDR3:
(SEQ ID NO: 12)
GGYDGSPWLSY;
and
a light chain variable region (abbreviated
herein as VL) comprising the following
three CDRs:
VL CDR1:
(SEQ ID NO: 13)
KASQDINSYLS;
VL CDR2:
(SEQ ID NO: 14)
RANRLVD;
VL CDR3:
(SEQ ID NO: 15)
LQYDEFPFT.

In some embodiments, the isolated antibody, or binding fragment thereof, that specifically binds to AGR2 comprises:
a VH comprising or consisting of a sequence at least 75%, 80%, 85%, 90%, 95%, 96%, 97%, 98%, 99% identical to SEQ ID NO: 16; and a VL comprising or consisting of a sequence at least 75%, 80%, 85%, 90%, 95%, 96%, 97%, 98%, 99% identical to SEQ ID NO: 19; or
a VH comprising or consisting of a sequence at least 75%, 80%, 85%, 90%, 95%, 96%, 97%, 98%, 99% identical to SEQ ID NO: 17; and a VL comprising or consisting of a sequence at least 75%, 80%, 85%, 90%, 95%, 96%, 97%, 98%, 99% identical to SEQ ID NO: 19; or
a VH comprising or consisting of a sequence at least 75%, 80%, 85%, 90%, 95%, 96%, 97%, 98%, 99% identical to SEQ ID NO: 16; and a VL comprising or consisting of a sequence at least 75%, 80%, 85%, 90%, 95%, 96%, 97%, 98%, 99% identical to SEQ ID NO: 20; or
a VH comprising or consisting of a sequence at least 75%, 80%, 85%, 90%, 95%, 96%, 97%, 98%, 99% identical to SEQ ID NO: 17; and a VL comprising or consisting of a sequence at least 75%, 80%, 85%, 90%, 95%, 96%, 97%, 98%, 99% identical to SEQ ID NO: 20; or
a VH comprising or consisting of a sequence at least 75%, 80%, 85%, 90%, 95%, 96%, 97%, 98%, 99% identical to SEQ ID NO: 18; and a VL comprising or consisting of a sequence at least 75%, 80%, 85%, 90%, 95%, 96%, 97%, 98%, 99% identical to SEQ ID NO: 21.

In some embodiments, the isolated antibody, or binding fragment thereof, that specifically binds to AGR2 comprises:
a heavy chain variable region (VH) comprising framework regions sharing at least 75%, 80%, 85%, 90%, 95%, 96%, 97%, 98%, 99% sequence identity with the framework regions of SEQ ID NO: 16; and a light chain variable region (VL) comprising framework regions sharing at least 75%, 80%, 85%, 90%, 95%, 96%, 97%, 98%, 99% sequence identity with the framework regions of SEQ ID NO: 19; or
a heavy chain variable region (VH) comprising framework regions sharing at least 75%, 80%, 85%, 90%, 95%, 96%, 97%, 98%, 99% sequence identity with the framework regions of SEQ ID NO: 17; and a light chain variable region (VL) comprising framework regions sharing at least 75%, 80%, 85%, 90%, 95%, 96%, 97%, 98%, 99% sequence identity with the framework regions of SEQ ID NO: 19; or
a heavy chain variable region (VH) comprising framework regions sharing at least 75%, 80%, 85%, 90%, 95%, 96%, 97%, 98%, 99% sequence identity with the framework regions of SEQ ID NO: 16; and a light chain variable region (VL) comprising framework regions sharing at least 75%, 80%, 85%, 90%, 95%, 96%, 97%, 98%, 99% sequence identity with the framework regions of SEQ ID NO: 20; or
a heavy chain variable region (VH) comprising framework regions sharing at least 75%, 80%, 85%, 90%, 95%, 96%, 97%, 98%, 99% sequence identity with the framework regions of SEQ ID NO: 17; and a light chain variable region (VL) comprising framework regions sharing at least 75%, 80%, 85%, 90%, 95%, 96%, 97%, 98%, 99% sequence identity with the framework regions of SEQ ID NO: 20; or
a heavy chain variable region (VH) comprising framework regions sharing at least 75%, 80%, 85%, 90%, 95%, 96%, 97%, 98%, 99% sequence identity with the framework regions of SEQ ID NO: 18; and a light chain variable region (VL) comprising framework regions sharing at least 75%, 80%, 85%, 90%, 95%, 96%, 97%, 98%, 99% sequence identity with the framework regions of SEQ ID NO: 21.

Preferably, the isolated antibody, or binding fragment thereof, that specifically binds to AGR2 of the invention comprises:
a VH comprising or consisting of the sequence SEQ ID NO: 16 and a VL comprising or consisting of the sequence SEQ ID NO: 19; or
a VH comprising or consisting of the sequence SEQ ID NO: 17 and a VL comprising or consisting of the sequence SEQ ID NO: 19; or
a VH comprising or consisting of the sequence SEQ ID NO: 16 and a VL comprising or consisting of the sequence SEQ ID NO: 20; or
a VH comprising or consisting of the sequence SEQ ID NO: 17 and a VL comprising or consisting of the sequence SEQ ID NO: 20; or
a VH comprising or consisting of the sequence SEQ ID NO: 18 and a VL comprising or consisting of the sequence SEQ ID NO: 21.

The fragment crystallizable (Fc) region of the isolated antibody that specifically binds to AGR2 may comprise at least one mutation reducing the antibody effector functions.

In some embodiments the isolated antibody that specifically binds to AGR2 is a Fc-silenced antibody.

As used herein “Fc-silenced antibody” refers to an antibody bearing a modification in the immunoglobulin Fc region in order to eliminate the binding of immunoglobulin Fc to Fc gamma receptors (FcγR).

As used herein “EU numbering” refers to the numbering of the constant domains of the heavy and light chains of the human gamma G1 immunoglobulin (IgG1) as defined by Edelman et al. (Proc Natl Acad Sci USA, 63(1):78-85, 1969). In this numbering, the first amino acid residue of the constant domain of the heavy chain is an alanine residue and is numbered at position 118, while the first amino acid residue of the constant domain of the light kappa chain is an arginine residue and is numbered at position 108.

In some embodiments, the isolated antibody that specifically binds to AGR2 of the present invention comprises a heavy chain constant region of an IgG1 and a heavy chain constant region sequence comprising mutations consisting of the leucine residue at position 234 in the EU numbering (corresponding to position 117 of SEQ ID NO:22) being substituted by an alanine residue, and the leucine residue at position 235 in the EU numbering (corresponding to position 118 of SEQ ID NO:22) being substituted by an alanine residue (L234A and L235A).

In some embodiments, the isolated antibody that specifically binds to AGR2 of the present invention comprises a heavy chain constant region of an IgG1 and a heavy chain constant region sequence comprising mutations consisting of the leucine residue at position 234 in the EU numbering (corresponding to position 117 of SEQ ID NO:22) being substituted by a phenylalanine residue, the leucine residue at position 235 in the EU numbering (corresponding to position 118 of SEQ ID NO:22) being substituted by a glutamic acid residue, and the proline residue at position 331 in the EU numbering (corresponding to position 214 of SEQ ID NO:22) being substituted by a serine residue (L234F, L235E and P331S).

In some embodiments, the isolated antibody that specifically binds to AGR2 of the present invention comprises a heavy chain constant region of an IgG4.

In some embodiments, the isolated antibody that specifically binds to AGR2 of the invention comprises:
a heavy chain constant region having a sequence comprising or consisting of sequence SEQ ID NO: 22, or a sequence at least 70%, 75%, 80%, 85%, 90%, 95%, 96%, 97%, 98%, 99% identical to SEQ ID NO: 22; and
a light chain constant region having a sequence comprising or consisting of sequence SEQ ID NO: 26, or a sequence at least 70%, 75%, 80%, 85%, 90%, 95%, 96%, 97%, 98%, 99% identical to SEQ ID NO: 26.

In some embodiments, the isolated antibody that specifically binds to AGR2 of the invention comprises:
a heavy chain constant region having a sequence comprising or consisting of sequence SEQ ID NO: 22; and
a light chain constant region having a sequence comprising or consisting of sequence SEQ ID NO: 26.

In some embodiments, the isolated antibody that specifically binds to AGR2 of the invention comprises:
a heavy chain constant region having a sequence comprising or consisting of sequence SEQ ID NO: 23, or a sequence at least 70%, 75%, 80%, 85%, 90%, 95%, 96%, 97%, 98%, 99% identical to SEQ ID NO: 23; and
a light chain constant region having a sequence comprising or consisting of sequence SEQ ID NO: 26, or a sequence at least 70%, 75%, 80%, 85%, 90%, 95%, 96%, 97%, 98%, 99% identical to SEQ ID NO: 26.

In some embodiments, the isolated antibody that specifically binds to AGR2 of the invention comprises:
a heavy chain constant region having a sequence comprising or consisting of sequence SEQ ID NO: 23; and
a light chain constant region having a sequence comprising or consisting of sequence SEQ ID NO: 26.

In some embodiments, the isolated antibody that specifically binds to AGR2 of the invention comprises:
a heavy chain constant region having a sequence comprising or consisting of sequence SEQ ID NO: 24, or a sequence at least 70%, 75%, 80%, 85%, 90%, 95%, 96%, 97%, 98%, 99% identical to SEQ ID NO: 24; and
a light chain constant region having a sequence comprising or consisting of sequence SEQ ID NO: 26, or a sequence at least 70%, 75%, 80%, 85%, 90%, 95%, 96%, 97%, 98%, 99% identical to SEQ ID NO: 26.

In some embodiments, the isolated antibody that specifically binds to AGR2 of the invention comprises:
a heavy chain constant region having a sequence comprising or consisting of sequence SEQ ID NO: 24; and
a light chain constant region having a sequence comprising or consisting of sequence SEQ ID NO: 26.

In some embodiments, the isolated antibody that specifically binds to AGR2 of the invention comprises:
a heavy chain constant region having a sequence comprising or consisting of sequence SEQ ID NO: 25, or a sequence at least 70%, 75%, 80%, 85%, 90%, 95%, 96%, 97%, 98%, 99% identical to SEQ ID NO: 25; and
a light chain constant region having a sequence comprising or consisting of sequence SEQ ID NO: 26, or a sequence at least 70%, 75%, 80%, 85%, 90%, 95%, 96%, 97%, 98%, 99% identical to SEQ ID NO: 26.

In some embodiments, the isolated antibody that specifically binds to AGR2 of the invention comprises:
a heavy chain constant region having a sequence comprising or consisting of sequence SEQ ID NO: 25; and
a light chain constant region having a sequence comprising or consisting of sequence SEQ ID NO: 26.

In some embodiments, the isolated antibody that specifically binds to AGR2 comprises:
a heavy chain having a sequence consisting of any one of sequence SEQ ID NO: 27-32, or a sequence at least 75%, 80%, 85%, 90%, 95%, 96%, 97%, 98%, 99% identical to one of SEQ ID NO: 27-32; and
a light chain having a sequence consisting of sequence SEQ ID NO: 33-35, or a sequence at least 75%, 80%, 85%, 90%, 95%, 96%, 97%, 98%, 99% identical to SEQ ID NO: 33-35.

In some embodiments, the isolated antibody that specifically binds to AGR2 comprises:
a heavy chain having a sequence consisting of sequence SEQ ID NO: 27 or SEQ ID NO: 30; and
a light chain having a sequence consisting of sequence SEQ ID NO: 33.

In some embodiments, the isolated antibody that specifically binds to AGR2 comprises:
a heavy chain having a sequence consisting of sequence SEQ ID NO: 28 or SEQ ID NO: 31; and
a light chain having a sequence consisting of sequence SEQ ID NO: 33.

In some embodiments, the isolated antibody that specifically binds to AGR2 comprises:
a heavy chain having a sequence consisting of sequence SEQ ID NO: 27 or SEQ ID NO: 30; and
a light chain having a sequence consisting of sequence SEQ ID NO: 34.

In some embodiments, the isolated antibody that specifically binds to AGR2 comprises:
a heavy chain having a sequence consisting of sequence SEQ ID NO: 28 or SEQ ID NO: 31; and
a light chain having a sequence consisting of sequence SEQ ID NO: 34.

In some embodiments, the isolated antibody that specifically binds to AGR2 comprises:
a heavy chain having a sequence consisting of sequence SEQ ID NO: 29 or SEQ ID NO: 32; and
a light chain having a sequence consisting of sequence SEQ ID NO: 35.

The isolated antibody, or binding fragment thereof, of the invention possesses anti-inflammatory functions. In particular, the isolated antibody, or binding fragment thereof, of the invention can inhibit the recruitment of monocytes induced by eAGR2.

The isolated antibody, or binding fragment thereof, of the invention possesses anti-fibrotic functions. In particular, the isolated antibody, or binding fragment thereof, of the invention can block the differentiation of fibroblasts into myofibroblasts.

The isolated antibody, or binding fragment thereof, of the invention possesses reduced effector functions. In particular, the isolated antibody, or binding fragment thereof, of the invention exhibits reduced binding to Fc receptors and/or reduced binding to complement components.

As used herein the term “reduced effector functions” for instance refers to reduced antibody-dependent cellular cytotoxicity (ADCC) and reduced complement dependent cytotoxicity (CDC).

Thus, the isolated antibody, or binding fragment thereof, of the invention has the following (non-exhaustive) advantages:
it specifically binds to AGR2 with a high affinity and does not cross-react with AGR3,
it specifically binds to AGR2 of various species (for instance human, mouse, dog, macaque, rat . . . ),
it specifically binds to both soluble AGR2 protein, to membrane-bound AGR2 protein, as well as to intracellular AGR2 protein,
it specifically binds to both the monomeric form and the dimeric form of AGR2,
it recognizes an epitope including at least one amino acid residue located in the catalytic site of AGR2,
it displays anti-inflammatory and/or anti-fibrotic activities,
it displays anti-proliferative and/or anti-metastatic activities, and
it preserves the mucus-secreting cells and/or is involved in the regeneration of the epithelium.

The isolated antibody, or binding fragment thereof, of the invention also displays improved characteristics in comparison to other anti-AGR2 antibodies, in particular in comparison to Agtuzumab, including e.g.:
easier production and better yield (typically, between 0.11 to 0.25 compared to 0.05 for Agtuzumab);
the epitope bound by the isolated antibody, or binding fragment thereof, of the invention is located in the catalytic domain of the human AGR2 protein, while the epitope bound by Agtuzumab is not located in a functional domain, suggesting that the isolated antibody, or binding fragment thereof, of the invention may be able to inhibit AGR2 catalytic activity, while Agtuzumab may not;
better binding capacity (typically having an EC50 between 520 to 2373 pM, compared to 3073 pM for Agtuzumab);
better affinity (typically having a K_{D} value under 150 pM compared to nanomolar range for Agtuzumab);
lower Kdapp (typically having 0.62 nM compared to 1.3 nM for Agtuzumab);
better thermal stability (higher melting temperature) (typically, from 80.36 to 83.28° C. compared to 76.28° C. for Agtuzumab); and
better inhibition of AGR2-induced monocytes migration.

Another object of the invention is an isolated nucleic acid encoding an isolated antibody, or binding fragment thereof, that specifically binds to AGR2 according to the present invention.

An “isolated nucleic acid”, as used herein, is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

Preferably, the isolated nucleic acid is purified.

For instance, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A260/280)), and most preferably greater than 96%, 97%, 98% or 99% by weight; or
(2) homogeneity as shown by agarose gel electrophoresis and using an intercalating agent such as ethidium bromide, SYBR Green, GelGreen or the like.

In some embodiments, the nucleic acid or set of nucleic acids according to the present invention comprises or consists of:
a sequence encoding the VH of the antibody, or binding fragment thereof, according to the present invention; and
a sequence encoding the VL of the antibody, or binding fragment thereof, according to the present invention.

In some embodiments, the nucleic acid or set of nucleic acids according to the present invention comprises or consists of:
a sequence encoding the heavy chain of the antibody according to the present invention; and
a sequence encoding the light chain of the antibody according to the present invention.

In some embodiments, the nucleic acid or set of nucleic acids according to the present invention comprises or consists of:
a sequence encoding the VH of the antibody, or binding fragment thereof, according to the present invention, said sequence being SEQ ID NO: 16 or any sequence sharing at least 70%, 75%, 80%, 85%, 90%, 95%, 96%, 97%, 98%, 99% or more identity with SEQ ID NO: 16 and a sequence encoding the VL of the antibody, or binding fragment thereof, according to the present invention, said sequence being SEQ ID NO: 19 or any sequence sharing at least 70%, 75%, 80%, 85%, 90%, 95%, 96%, 97%, 98%, 99% or more identity with SEQ ID NO: 19; or
a sequence encoding the VH of the antibody, or binding fragment thereof, according to the present invention, said sequence being SEQ ID NO: 17 or any sequence sharing at least 70%, 75%, 80%, 85%, 90%, 95%, 96%, 97%, 98%, 99% or more identity with SEQ ID NO: 17 and a sequence encoding the VL of the antibody, or binding fragment thereof, according to the present invention, said sequence being SEQ ID NO: 19 or any sequence sharing at least 70%, 75%, 80%, 85%, 90%, 95%, 96%, 97%, 98%, 99% or more identity with SEQ ID NO: 19; or
a sequence encoding the VH of the antibody, or binding fragment thereof, according to the present invention, said sequence being SEQ ID NO: 16 or any sequence sharing at least 70%, 75%, 80%, 85%, 90%, 95%, 96%, 97%, 98%, 99% or more identity with SEQ ID NO: 16 and a sequence encoding the VL of the antibody, or binding fragment thereof, according to the present invention, said sequence being SEQ ID NO: 20 or any sequence sharing at least 70%, 75%, 80%, 85%, 90%, 95%, 96%, 97%, 98%, 99% or more identity with SEQ ID NO: 20; or
a sequence encoding the VH of the antibody, or binding fragment thereof, according to the present invention, said sequence being SEQ ID NO: 17 or any sequence sharing at least 70%, 75%, 80%, 85%, 90%, 95%, 96%, 97%, 98%, 99% or more identity with SEQ ID NO: 17 and a sequence encoding the VL of the antibody, or binding fragment thereof, according to the present invention, said sequence being SEQ ID NO: 20 or any sequence sharing at least 70%, 75%, 80%, 85%, 90%, 95%, 96%, 97%, 98%, 99% or more identity with SEQ ID NO: 20; or
a sequence encoding the VH of the antibody, or binding fragment thereof, according to the present invention, said sequence being SEQ ID NO: 18 or any sequence sharing at least 70%, 75%, 80%, 85%, 90%, 95%, 96%, 97%, 98%, 99% or more identity with SEQ ID NO: 18 and a sequence encoding the VL of the antibody, or binding fragment thereof, according to the present invention, said sequence being SEQ ID NO: 21 or any sequence sharing at least 70%, 75%, 80%, 85%, 90%, 95%, 96%, 97%, 98%, 99% or more identity with SEQ ID NO: 21.

In some embodiments, the nucleic acid or set of nucleic acids according to the present invention comprises or consists of:
a sequence encoding the heavy chain of the antibody according to the present invention, said sequence being SEQ ID NO: 36 or any sequence sharing at least 70%, 75%, 80%, 85%, 90%, 95%, 96%, 97%, 98%, 99% or more identity with SEQ ID NO: 36; and a sequence encoding the light chain of the antibody according to the present invention, said sequence being SEQ ID NO: 37 or any sequence sharing at least 70%, 75%, 80%, 85%, 90%, 95%, 96%, 97%, 98%, 99% or more identity with SEQ ID NO: 37; or
a sequence encoding the heavy chain of the antibody according to the present invention, said sequence being SEQ ID NO: 38 or any sequence sharing at least 70%, 75%, 80%, 85%, 90%, 95%, 96%, 97%, 98%, 99% or more identity with SEQ ID NO: 38; and a sequence encoding the light chain of the antibody according to the present invention, said sequence being SEQ ID NO: 39 or any sequence sharing at least 70%, 75%, 80%, 85%, 90%, 95%, 96%, 97%, 98%, 99% or more identity with SEQ ID NO: 39; or
a sequence encoding the heavy chain of the antibody according to the present invention, said sequence being SEQ ID NO: 40 or SEQ ID NO: 42 or any sequence sharing at least 70%, 75%, 80%, 85%, 90%, 95%, 96%, 97%, 98%, 99% or more identity with SEQ ID NO: 40 or SEQ ID NO: 42; and a sequence encoding the light chain of the antibody according to the present invention, said sequence being SEQ ID NO: 41 or SEQ ID NO: 43 or any sequence sharing at least 70%, 75%, 80%, 85%, 90%, 95%, 96%, 97%, 98%, 99% or more identity with SEQ ID NO: 41 or SEQ ID NO: 43; or
a sequence encoding the heavy chain of the antibody according to the present invention, said sequence being SEQ ID NO: 44 or any sequence sharing at least 70%, 75%, 80%, 85%, 90%, 95%, 96%, 97%, 98%, 99% or more identity with SEQ ID NO: 44; and a sequence encoding the light chain of the antibody according to the present invention, said sequence being SEQ ID NO: 45 or any sequence sharing at least 70%, 75%, 80%, 85%, 90%, 95%, 96%, 97%, 98%, 99% or more identity with SEQ ID NO: 45; or
a sequence encoding the heavy chain of the antibody according to the present invention, said sequence being SEQ ID NO: 46 or any sequence sharing at least 70%, 75%, 80%, 85%, 90%, 95%, 96%, 97%, 98%, 99% or more identity with SEQ ID NO: 46; and a sequence encoding the light chain of the antibody according to the present invention, said sequence being SEQ ID NO: 47 or any sequence sharing at least 70%, 75%, 80%, 85%, 90%, 95%, 96%, 97%, 98%, 99% or more identity with SEQ ID NO: 47.

Another object of the present invention is an expression vector comprising the nucleic acid encoding the isolated antibody, or binding fragment thereof, that specifically binds to AGR2 according to the invention.

In some embodiments, the expression vector or set of expression vectors according to the present invention comprises:
a sequence encoding the VH of the antibody, or binding fragment thereof, according to the present invention, preferably operably linked to regulatory elements;
a sequence encoding the VL of the antibody, or binding fragment thereof, according to the present invention, preferably operably linked to regulatory elements.

In some embodiments, the expression vector according to the present invention comprises:
a sequence encoding the VH of the antibody, or binding fragment thereof, according to the present invention, said sequence being SEQ ID NO: 16 or any sequence sharing at least 70%, 75%, 80%, 85%, 90%, 95%, 96%, 97%, 98%, 99% or more identity with SEQ ID NO: 16 and a sequence encoding the VL of the antibody, or binding fragment thereof, according to the present invention, said sequence being SEQ ID NO: 19 or any sequence sharing at least 70%, 75%, 80%, 85%, 90%, 95%, 96%, 97%, 98%, 99% or more identity with SEQ ID NO: 19; or
a sequence encoding the VH of the antibody, or binding fragment thereof, according to the present invention, said sequence being SEQ ID NO: 17 or any sequence sharing at least 70%, 75%, 80%, 85%, 90%, 95%, 96%, 97%, 98%, 99% or more identity with SEQ ID NO: 17 and a sequence encoding the VL of the antibody, or binding fragment thereof, according to the present invention, said sequence being SEQ ID NO: 19 or any sequence sharing at least 70%, 75%, 80%, 85%, 90%, 95%, 96%, 97%, 98%, 99% or more identity with SEQ ID NO: 19; or
a sequence encoding the VH of the antibody, or binding fragment thereof, according to the present invention, said sequence being SEQ ID NO: 16 or any sequence sharing at least 70%, 75%, 80%, 85%, 90%, 95%, 96%, 97%, 98%, 99% or more identity with SEQ ID NO: 16 and a sequence encoding the VL of the antibody, or binding fragment thereof, according to the present invention, said sequence being SEQ ID NO: 20 or any sequence sharing at least 70%, 75%, 80%, 85%, 90%, 95%, 96%, 97%, 98%, 99% or more identity with SEQ ID NO: 20; or
a sequence encoding the VH of the antibody, or binding fragment thereof, according to the present invention, said sequence being SEQ ID NO: 17 or any sequence sharing at least 70%, 75%, 80%, 85%, 90%, 95%, 96%, 97%, 98%, 99% or more identity with SEQ ID NO: 17 and a sequence encoding the VL of the antibody, or binding fragment thereof, according to the present invention, said sequence being SEQ ID NO: 20 or any sequence sharing at least 70%, 75%, 80%, 85%, 90%, 95%, 96%, 97%, 98%, 99% or more identity with SEQ ID NO: 20; or
a sequence encoding the VH of the antibody, or binding fragment thereof, according to the present invention, said sequence being SEQ ID NO: 18 or any sequence sharing at least 70%, 75%, 80%, 85%, 90%, 95%, 96%, 97%, 98%, 99% or more identity with SEQ ID NO: 18 and a sequence encoding the VL of the antibody, or binding fragment thereof, according to the present invention, said sequence being SEQ ID NO: 21 or any sequence sharing at least 70%, 75%, 80%, 85%, 90%, 95%, 96%, 97%, 98%, 99% or more identity with SEQ ID NO: 21.

In some embodiments, the expression vector or set of expression vectors according to the present invention comprises:
a sequence encoding the heavy chain of the isolated antibody that specifically binds to AGR2 according to the invention, preferably operably linked to regulatory elements; and
a sequence encoding the light chain of the isolated antibody that specifically binds to AGR2 according to the invention, preferably operably linked to regulatory elements.

In some embodiments, the expression vector according to the present invention comprises:
a sequence encoding the heavy chain of the antibody according to the present invention, said sequence being SEQ ID NO: 36 or any sequence sharing at least 70%, 75%, 80%, 85% 90%, 95%, 96%, 97%, 98%, 99% or more identity with SEQ ID NO: 36, preferably operably linked to regulatory elements; and a sequence encoding the light chain of the antibody according to the present invention, said sequence being SEQ ID NO: 37 or any sequence sharing at least 70%, 75%, 80%, 85% 90%, 95%, 96%, 97%, 98%, 99% or more identity with SEQ ID NO: 37, preferably operably linked to regulatory elements; or
a sequence encoding the heavy chain of the antibody according to the present invention, said sequence being SEQ ID NO: 38 or any sequence sharing at least 70%, 75%, 80%, 85% 90%, 95%, 96%, 97%, 98%, 99% or more identity with SEQ ID NO: 38, preferably operably linked to regulatory elements; and a sequence encoding the light chain of the antibody according to the present invention, said sequence being SEQ ID NO: 39 or any sequence sharing at least 70%, 75%, 80%, 85% 90%, 95%, 96%, 97%, 98%, 99% or more identity with SEQ ID NO: 39, preferably operably linked to regulatory elements; or
a sequence encoding the heavy chain of the antibody according to the present invention, said sequence being SEQ ID NO: 40 or SEQ ID NO: 42 or any sequence sharing at least 70%, 75%, 80%, 85% 90%, 95%, 96%, 97%, 98%, 99% or more identity with SEQ ID NO: 40 or SEQ ID NO: 42, preferably operably linked to regulatory elements; and a sequence encoding the light chain of the antibody according to the present invention, said sequence being SEQ ID NO: 41 or SEQ ID NO: 43 or any sequence sharing at least 70%, 75%, 80%, 85% 90%, 95%, 96%, 97%, 98%, 99% or more identity with SEQ ID NO: 41 or SEQ ID NO: 43, preferably operably linked to regulatory elements; or
a sequence encoding the heavy chain of the antibody according to the present invention, said sequence being SEQ ID NO: 44 or any sequence sharing at least 70%, 75%, 80%, 85% 90%, 95%, 96%, 97%, 98%, 99% or more identity with SEQ ID NO: 44, preferably operably linked to regulatory elements; and a sequence encoding the light chain of the antibody according to the present invention, said sequence being SEQ ID NO: 45 or any sequence sharing at least 70%, 75%, 80%, 85% 90%, 95%, 96%, 97%, 98%, 99% or more identity with SEQ ID NO: 45, preferably operably linked to regulatory elements; or
a sequence encoding the heavy chain of the antibody according to the present invention, said sequence being SEQ ID NO: 46 or any sequence sharing at least 70%, 75%, 80%, 85% 90%, 95%, 96%, 97%, 98%, 99% or more identity with SEQ ID NO: 46, preferably operably linked to regulatory elements; and a sequence encoding the light chain of the antibody according to the present invention, said sequence being SEQ ID NO: 47 or any sequence sharing at least 70%, 75%, 80%, 85% 90%, 95%, 96%, 97%, 98%, 99% or more identity with SEQ ID NO: 47, preferably operably linked to regulatory elements.

In some embodiments, the expression vector according to the present invention is monocistronic.

By “monocistronic”, it is meant that a single nucleic acid encoding a single protein is expressed in a single expression vector.

In some embodiments, the expression vector according to the present invention is polycistronic.

By “polycistronic”, it is meant that at least two or more nucleic acids, each encoding a single protein, are expressed in a single expression vector.

Another object of the invention is a cell comprising the nucleic acid encoding the isolated antibody, or binding fragment thereof, that specifically binds to AGR2 according to the invention, or the expression vector comprising the nucleic acid encoding the isolated antibody, or binding fragment thereof, that specifically binds to AGR2 according to the invention.

In some embodiments, the cell is an Escherichia coli (E. coli) cell. In other embodiments, the cell is a Chinese hamster ovary (CHO) cell.

Another object of the invention is a method of producing and purifying the isolated antibody, or binding fragment thereof, that specifically binds to AGR2 according to the invention.

In some embodiments, the method comprises:
Culturing host cells transformed with the nucleic acid or expression vector according to the present invention, under conditions suitable for expression of the isolated antibody, or binding fragment thereof, that specifically binds to AGR2;
Recovering the expressed antibody, or binding fragment thereof, that specifically binds to AGR2.

This recombinant process can be used for large scale production of antibodies or binding fragments thereof, including monoclonal antibodies intended for in vitro, ex vivo and/or in vivo therapeutic and/or diagnostic uses.

These processes are well-known in the art (Subramanian (Ed.), 2004. Antibodies (1st ed., Vol. 1: Production and Purification). New York, NY: Springer US).

In some embodiments, the expressed antibody, or binding fragment thereof, is further purified.

Methods to purify the antibody, or binding fragment thereof, according to the present invention are well-known in the art (Subramanian (Ed.), 2004. Antibodies (1st ed., Vol. 1: Production and Purification). New York, NY: Springer US), and include, without limitation, chromatography, preferably by affinity chromatography, more preferably by affinity chromatography on protein L agarose.

In some embodiments, the antibody, or binding fragment thereof, according to the present invention is an immunoconjugate.

In some embodiments, the immunoconjugate according to the present invention is an antibody, or binding fragment thereof, conjugated to a therapeutic moiety, i.e., a drug. In some embodiments, the therapeutic moiety is selected from a cytokine, an immunomodulator, an immunosuppressant, a cytotoxin, a chemotherapeutic agent, a lytic peptide and a radioisotope. Such conjugates are referred to herein as an “antibody drug conjugates” or “ADCs”.

In some embodiments, the immunoconjugate according to the present invention is a labelled antibody, or binding fragment thereof.

By “labeled”, it is meant that the isolated antibody, or binding fragment thereof, has at least one element, isotope or chemical compound conjugated or attached to it, enabling for example the detection of said isolated antibody, or binding fragment thereof.

Examples of labels include, but are not limited to, luminescent dyes (also termed fluorophores or photodetectable labels), isotopic labels (also termed radioactive labels, radiolabels or heavy isotopes), contrast agents, magnetic labels, electric labels, thermal labels, and colored labels.

Another object of the present invention is a composition comprising, consisting essentially of or consisting of:
at least one isolated antibody, or binding fragment thereof, that specifically binds to AGR2 of the present invention;
at least one immunoconjugate as described herein;
at least one nucleic acid encoding the isolated antibody, or binding fragment thereof, that specifically binds to AGR2 as described herein;
at least one expression vector comprising such a nucleic acid; or at least one host cell comprising such an expression vector.

In some embodiments, said composition is a pharmaceutical composition and further comprises at least one pharmaceutically acceptable excipient.

Consequently, another object of the present invention is a pharmaceutical composition comprising, consisting essentially of or consisting of
at least one isolated antibody, or binding fragment thereof, that specifically binds to AGR2 of the present invention;
at least one immunoconjugate as described herein;
at least one nucleic acid encoding the isolated antibody, or binding fragment thereof, that specifically binds to AGR2 as described herein;
at least one expression vector comprising such a nucleic acid; or
at least one host cell comprising such an expression vector.
   and at least one pharmaceutically acceptable excipient.

As used herein, “consisting essentially of”, with reference to a composition of the invention, means that the at least one isolated anti-AGR2 antibody, or binding fragment thereof, is the only agent with a biologic or therapeutic activity within said composition.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a mammal, more preferably a human. For human administration, preparations should meet sterility, pyrogenicity, and general safety and purity standards as required by regulatory offices, such as, for example, FDA Office or EMA.

Examples of pharmaceutically acceptable excipients that may be used in the compositions of the present invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In some embodiments, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Another object of the present invention is a medicament comprising, consisting essentially of or consisting of
at least one isolated antibody, or binding fragment thereof, that specifically binds to AGR2 of the present invention;

at least one immunoconjugate as described herein;
at least one nucleic acid encoding the isolated antibody, or binding fragment thereof, that specifically binds to AGR2 as described herein;
at least one expression vector comprising such a nucleic acid; or
at least one host cell comprising such an expression vector.

Another object of the invention is a kit comprising
at least one isolated antibody, or binding fragment thereof, that specifically binds to AGR2 according to the present invention;
at least one immunoconjugate as described herein;
at least one nucleic acid encoding the isolated antibody, or binding fragment thereof, that specifically binds to AGR2 as described herein;
1 at least one expression vector comprising such a nucleic acid; or
at least one host cell comprising such an expression vector.
   and instructions for use.

By “kit” is intended any manufacture (e.g., a package or a container) comprising at least isolated antibody, or binding fragment thereof, that specifically binds to AGR2 according to the present invention. The kit may be promoted, distributed, or sold as a unit for performing the methods of the present invention.

AGR2 has been shown to be overexpressed in mucosal inflammatory diseases. Moreover, secretion of AGR2 monomers have been observed in inflammatory bowel disease, and specifically in Crohn's disease. Moreover, extracellular AGR2 displays pro-inflammatory and pro-fibrotic properties.

Therefore, AGR2 is a promising therapeutic target in the context of inflammatory diseases. Indeed, AGR2 blocking agents, such as e.g., anti-AGR2 antibodies, may act at two levels in the inflammatory process involved in mucosal inflammatory diseases: 1) by blocking local monocyte recruitment, and 2) by blocking fibroblast-to-myofibroblast differentiation and thus fibrosis installation.

The present invention further relates to an isolated antibody, or binding fragment thereof, that specifically binds to AGR2 as described herein, for use as a medicament.

Another object of the present invention is a nucleic acid encoding an isolated antibody, or binding fragment thereof, that specifically binds to AGR2 as described herein, or an expression vector comprising such a nucleic acid molecule, or a host cell comprising such an expression vector, for use as a medicament.

The present invention further relates to an isolated antibody, or binding fragment thereof, that specifically binds to AGR2 as described herein for use for treating and/or preventing mucosal inflammatory diseases in a subject in need thereof.

Another object of the present invention is a nucleic acid encoding an isolated antibody, or binding fragment thereof, that specifically binds to AGR2 as described herein, or an expression vector comprising such a nucleic acid, or a host cell comprising such an expression vector, or a composition as described herein, or a pharmaceutical composition as described herein, for use for treating and/or preventing mucosal inflammatory diseases in a subject in need thereof.

As used herein the term “inflammatory diseases” refers to a vast array of disorders and conditions that are characterized by inflammation. Symptoms of inflammatory disease can include chronic pain, swelling, redness, joint and muscle stiffness, loss of function and movement in the affected area. Inflammatory disorders refer to many heterogeneous conditions that may have the following pathophysiological characteristics:
an inflammatory response to an unidentified agent that involves different tissues and organs,
a response depending on genetic variability in the response characteristics of immune cells such as antigen-presenting cells, B and T lymphocytes,
production of autoantibodies (natural and pathogenic autoantibodies) to the exogenous or endogenous antigen,
production of antigen-specific inflammatory cells, such as lymphocytes and T-cells,
production and deposition of abnormal protein and other inflammatory products in different tissues eliciting further inflammatory and immune responses,
these responses include inflammation of vessels in the surrounding tissue (i.e., vasculitis) and/or
production of pro-inflammatory and anti-inflammatory mediators.

The persistence of the chronic inflammatory state is thought to reflect an imbalance between proinflammatory cytokines, such as TNF-alpha, IL-1, IL-6, and GM-CSF, and anti-inflammatory cytokines, including IL-10, and TGF-beta, whereupon a deficiency of anti-inflammatory cytokines would be consistent with a failure of immunoregulatory mechanisms. Chronic inflammatory changes may occur as a result of failure of regulatory T-cells to down-regulate the inflammatory process. Non-limiting examples of inflammatory diseases include allergy, asthma, autoimmune diseases, coeliac disease, glomerulonephritis, hepatitis, inflammatory bowel disease, preperfusion injury and transplant rejection.

As used herein, the term “mucosa” refers to the moist tissue lining body cavities which secretes mucous and covered with epithelium. Examples of mucosa tissue include, but are not limited to: oral mucosa (e.g., buccal and sublingual), nasal mucosa, eye mucosa, genital mucosa, rectal mucosa, lung mucosa, bronchial mucosa, gastric mucosa, intestinal mucosa, olfactory mucosa, uterine mucosa and esophageal mucosa.

As used herein the term “mucosal inflammatory diseases” refers to particular inflammatory diseases involving mucosal inflammation. The pathogenesis is unknown and is likely multifactorial, involving genetic susceptibility, environmental factors, microbiota, and immune system. Mucosal inflammation is characterized by atrophy of the squamous epithelium, vascular damage, ulceration and inflammatory infiltration, leading to fibrosis. It usually affects the mucous lining of the mouth, the gastrointestinal tract or the respiratory system. Non-limiting examples of mucosal inflammatory diseases include Crohn's disease, ulcerative colitis, primary sclerosing cholangitis, chronic pancreatitis, microscopic colitis, inflammatory bowel disease (IBD), irritable bowel syndrome, endometriosis, appendicitis, asthma, idiopathic pulmonary fibrosis, systemic sclerosis associated with interstitial lung disease and chronic obstructive pulmonary disease. For instance, inflammatory bowel diseases (IBD) comprise ulcerative colitis and Crohn's disease, which are characterized by idiopathic inflammation of the gastrointestinal tract.

“Crohn's disease”, as used herein, refers to a condition involving chronic inflammation of the gastrointestinal tract. Crohn's-related inflammation usually affects the intestines but may occur anywhere from the mouth to the anus. Crohn's disease differs from ulcerative colitis in that the inflammation extends through all layers of the intestinal wall and involves mesentery as well as lymph nodes. The disease is often discontinuous, i.e., severely diseased segments of bowel are separated from apparently disease-free areas. In Crohn's disease, the bowel wall also thickens which can lead to obstructions, and the development of fistulas and fissures are not uncommon. Crohn's disease may be one or more of several types of Crohn's disease, including without limitation: ileocolitis (affects the ileum and the large intestine), ileitis (affects the ileum), gastroduodenal Crohn's disease (inflammation in the stomach and the duodenum), jejunoileitis (spotty patches of inflammation in the jejunum), and Crohn's (granulomatous) colitis (only affects the large intestine).

“Ulcerative colitis”, as used herein, refers to a condition involving inflammation of the large intestine and rectum. In patients with ulcerative colitis, there is an inflammatory reaction primarily involving the colonic mucosa. The inflammation is typically uniform and continuous with no intervening areas of normal mucosa. Surface mucosal cells as well as crypt epithelium and submucosa are involved in an inflammatory reaction with neutrophil infiltration. Ultimately, this reaction typically progresses to epithelial damage and loss of epithelial cells resulting in multiple ulcerations, fibrosis, dysplasia and longitudinal retraction of the colon.

AGR2 is a marker of tumor aggressiveness expressed by many solid tumor types. Due to almost ubiquitous expression in solid tumors, expression in premalignant lesions and its involvement in metastatic disease, AGR2 protein represents a relevant target for cancer therapy. AGR2, in particular extracellular AGR2, is also implicated in drug resistance to chemotherapy, mesenchymal transition (EMT), angiogenesis, metastasis, and in cancer cell proliferation, migration and invasion.

Therefore, the present invention further relates to an isolated antibody, or binding fragment thereof, that specifically binds to AGR2 as described herein, for use for treating and/or preventing cancer in a subject in need thereof.

Another object of the present invention is a nucleic acid encoding an isolated antibody, or binding fragment thereof, that specifically binds to AGR2 as described herein, or an expression vector comprising such a nucleic acid, or a host cell comprising such an expression vector, or a composition as described herein, or a pharmaceutical composition as described herein, for use for treating and/or preventing cancer in a subject in need thereof.

As used herein, the term “cancer” has its general meaning in the art and in particular refers to a disease caused by an uncontrolled division of abnormal cells. The term “cancer” encompasses solid tumors and blood cancers, and encompasses both primary and metastatic cancers.

Examples of cancers include, but are not limited to, cancer cells from the bladder, blood, bone, bone marrow, brain, breast, colon, esophagus, gastrointestinal, gum, head, kidney, liver, lung, nasopharynx, neck, ovary, prostate, pancreatic, skin, stomach, testis, tongue, or uterus.

In some embodiments, said cancer is a tumor, such as, for example, a solid tumor.

In some embodiments, said cancer is an epithelial cancer.

In some embodiments, said cancer is a urogenital cancer. Examples of urogenital cancers include without being limited to adrenocortical carcinoma, bladder cancer, kidney cancer, penile cancer, prostate cancer, renal cancer, testicular cancer, urethral cancer, colorectal cancer, cervical cancer, ovarian cancer, uterine cancer, endometrial cancer, vaginal cancer, vulvar cancer, gestational trophoblastic disease (GTD), primary peritoneal cancer.

In some embodiments, said cancer is a blood cancer. In some embodiments, said cancer is a hematologic malignancy. Examples of hematologic cancers include, but are not limited to, Hodgkin's disease, non-Hodgkin's lymphoma (malignant lymphoma) and blood cancers such as e.g., acute or chronic myeloid leukemia, acute lymphoblastic leukemia, chronic lymphocytic leukemia, myeloproliferative diseases, multiple myeloma, myelodysplastic syndrome.

Examples of cancers include, but are not limited to, acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, Burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous T cell lymphoma, embryonal tumors, endometrial cancer, ependymoblastoma, ependymoma, esophageal cancer, esthesioneuroblastoma, Ewing sarcoma family of tumors, extracranial germ cell tumor, extragonadal germ cell tumor, extrahepatic bile duct cancer, eye cancer fibrous histiocytoma of bone and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone, medulloblastoma, medulloepithelioma, melanoma, Merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), myeloid leukemia, acute myeloid leukemia (AML), multiple myeloma, myeloproliferative disorders, nasal cavity and paranasal sinus cancer, nasopharyngeal cancer, neuroblastoma, non-Hodgkin lymphoma, non-small cell lung cancer, oral cancer, oral cavity cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, Sezary syndrome, small cell lung cancer, small intestine cancer, soft tissue sarcoma, squamous cell carcinoma, squamous neck cancer, stomach (gastric) cancer, supratentorial primitive neuroectodermal tumors, T cell lymphoma, cutaneous cancer, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenstrom macroglobulinemia, and Wilms Tumor, cholangiocarcinoma, head and neck squamous cell carcinoma, brain glioblastoma, adrenocortical carcinoma, bladder cancer, kidney cancer, renal cancer, colorectal cancer, cervical cancer, gestational trophoblastic disease (GTD), primary peritoneal cancer.

Preferably, the cancer to be prevented or treated with the isolated antibody, or binding fragment thereof, that specifically binds to AGR2 is selected from the group consisting of colon cancer, gastrointestinal cancer, prostate cancer, pancreatic cancer, oral cancer, breast cancer, lung cancer, ovarian cancer, thyroid cancer, cholangiocarcinoma, head and neck squamous cell carcinoma, brain glioblastoma, adrenocortical carcinoma, bladder cancer, kidney cancer, penile cancer, renal cancer, testicular cancer, urethral cancer, colorectal cancer, cervical cancer, uterine cancer, endometrial cancer, vaginal cancer, vulvar cancer, gestational trophoblastic disease (GTD), and primary peritoneal cancer.

The present invention further relates to a method for treating and/or preventing mucosal inflammatory diseases or cancers in a subject in need thereof, comprising administering to the subject an isolated antibody, or binding fragment thereof, that specifically binds to AGR2 as described herein, or a nucleic acid encoding an isolated antibody, or binding fragment thereof, that specifically binds to AGR2 as described herein, or an expression vector comprising such a nucleic acid, or a host cell comprising such an expression vector, or a composition as described herein, or a pharmaceutical composition as described herein.

The present invention further relates to the use of an isolated antibody, or binding fragment thereof, that specifically binds to AGR2 as described herein, for the manufacture of a medicament for the treatment and/or prevention of mucosal inflammatory diseases or cancers in a subject in need thereof.

The present invention also relates to the use of a nucleic acid encoding an isolated antibody, or binding fragment thereof, that specifically binds to AGR2 as described herein, or an expression vector comprising such a nucleic acid, or a host cell comprising such an expression vector, or a composition as described herein, or a pharmaceutical composition as described herein, for the manufacture of a medicament for the treatment and/or prevention of mucosal inflammatory diseases or cancers in a subject in need thereof.

The present invention further relates to the use of the isolated antibody, or binding fragment thereof, that specifically binds to AGR2 as described herein, for treating and/or preventing mucosal inflammatory diseases or cancers in a subject in need thereof.

The present invention also relates to the use of the nucleic acid encoding the isolated antibody, or binding fragment thereof, that specifically binds to AGR2 as described herein, or the expression vector comprising such nucleic acid, or the host cell comprising such expression vector, or the composition as described herein, or the pharmaceutical composition as described herein, for treating and/or preventing mucosal inflammatory diseases or cancers in a subject in need thereof.

In some embodiments, the isolated antibody, or binding fragment thereof, that specifically binds to AGR2 according to the present invention, the nucleic acid encoding an isolated antibody, or binding fragment thereof, that specifically binds to AGR2 as described herein, the expression vector comprising such a nucleic acid, the host cell comprising such an expression vector, the composition as described herein, or the pharmaceutical composition as described herein, may be used for treating and/or preventing mucosal inflammatory diseases or cancers in a subject in need thereof.

The present invention further relates to a method for reducing mucosal inflammation in a subject in need thereof, comprising administering to the subject an isolated antibody, or binding fragment thereof, that specifically binds to AGR2 as described herein.

The present invention also relates to a method for reducing mucosal inflammation in a subject in need thereof, comprising administering to the subject a nucleic acid encoding an isolated antibody, or binding fragment thereof, that specifically binds to AGR2 as described herein, or an expression vector comprising such a nucleic acid, or a host cell comprising such an expression vector, or a composition as described herein, or a pharmaceutical composition as described herein.

Examples of mucosal inflammatory diseases include, but are not limited to Crohn's disease, ulcerative colitis, primary sclerosing cholangitis, chronic pancreatitis, microscopic colitis, inflammatory bowel disease (IBD), endometriosis, appendicitis, inflammatory bowel syndrome, idiopathic pulmonary fibrosis, systemic sclerosis, in particular systemic sclerosis associated with interstitial lung disease, asthma and chronic obstructive pulmonary disease.

The present invention further relates to a method for reducing the pro-inflammatory activity and/or the pro-fibrotic activity of extracellular AGR2 in a subject in need thereof, said method comprising administering to said subject a therapeutically effective amount of an isolated antibody, or binding fragment thereof, that specifically binds to AGR2 of the invention.

The present invention also relates to a method for reducing the pro-inflammatory activity and/or the pro-fibrotic activity of extracellular AGR2 in a subject in need thereof, said method comprising administering to said subject a therapeutically effective amount of a nucleic acid encoding an isolated antibody, or binding fragment thereof, that specifically binds to AGR2 as described herein, or an expression vector comprising such a nucleic acid, or a host cell comprising such an expression vector, or a composition as described herein, or a pharmaceutical composition as described herein

For use in administration to a subject, the composition, pharmaceutical composition or medicament will be formulated.

In some embodiments, the composition, pharmaceutical composition or medicament according to the present invention is administered parenterally, orally, by inhalation, spray, rectally, nasally, or via an implanted reservoir.

In some embodiments, the composition, pharmaceutical composition or medicament is administered by injection, including, without limitation, subcutaneous, intravenous, intramuscular, intra-articular, intra-synovial, intra-sternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

In some embodiments, the isolated anti-AGR2 antibody, or binding fragment thereof, composition, pharmaceutical composition or medicament according to the present invention, is to be administered to the subject in need thereof in a therapeutically effective amount.

It will be however understood that the total daily usage of the isolated anti-AGR2 antibody, or binding fragment thereof, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the isolated antibody, or binding fragment thereof, that specifically binds to AGR2 employed; the age, body weight, general health, sex and diet of the subject; the time of administration, route of administration, and rate of excretion of the specific therapeutic agent employed; the duration of the treatment; drugs used in combination or coincidental with the specific therapeutic agent employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In some embodiments, the daily dosage of the isolated antibody, or binding fragment thereof, that specifically binds to AGR2 may be varied over a wide range from 0.01 to 1000 mg per adult per day. Compositions may contain 0.01, 0.05, 0.1, 0.5, 1.0, 2.5, 5.0, 10.0, 15.0, 25.0, 50.0, 100, 250, and 500 mg of the active ingredient for the symptomatic adjustment of the dosage to the subject to be treated. A pharmaceutical composition or medicament typically contains from about 0.01 mg to about 500 mg of active ingredient. A therapeutically effective amount of the drug is ordinarily supplied at a dosage level from 0.0002 mg/kg to about 20 mg/kg of body weight per day. For example, an antibody, or binding fragment thereof, present in a composition, pharmaceutical composition or medicament of this invention can be supplied at a concentration ranging from 1 mg/mL to about 100 mg/mL, such as, for example, at a concentration of 1 mg/mL, 5 mg/mL, 10 mg/mL, 50 mg/mL or 100 mg/mL. In some embodiments, the isolated antibody, or binding fragment thereof, that specifically binds to AGR2 is supplied at a concentration of about 10 mg/mL in either 100 mg (10 mL) or 500 mg (50 mL) single-use vials. It will be appreciated that these dosages are exemplary and that an optimal dosage can be adapted taking into account the affinity and tolerability of the particular therapeutic agent that must be determined in clinical trials.

In some embodiments, the isolated antibody, or binding fragment thereof, nucleic acid, expression vector, composition, pharmaceutical composition or medicament according to the present invention is to be administered before, concomitantly with or after a therapeutic drug.

Some examples of therapeutic drugs suitable for co-administration with the isolated antibody, or binding fragment thereof, nucleic acid, expression vector, composition, pharmaceutical composition or medicament according to the present invention include, without limitation, immunosuppressants, cytokines, immune modulators.

It will be understood by the one skilled in the art that the co-administration of the isolated antibody, or binding fragment thereof, nucleic acid, expression vector, composition, pharmaceutical composition or medicament according to the present invention with a particular therapeutic drug, which may be chosen among those recited herein but without being limited thereto, will depend on the disease or condition to be prevented and/or treated.

Examples of immunosuppressants include, without limitation, corticosteroids, mTOR inhibitors such as, e.g., sirolimus, everolimus, ridaforolimus, temsirolimus, umirolimus and zotarolimus; IL-1 receptor antagonists such as, e.g., anakinra; antimetabolites such as, e.g., azathioprine, leflunomide, methotrexate, mycophenolic acid and teriflunomide; IMiDs such as, e.g., apremilast, lenalidomide, pomalidomide and thalidomide; antibodies such as, e.g., vedolizumab, eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, zolimomab aritox, atorolimumab, cedelizumab, fontolizumab, maslimomab, morolimumab, pexelizumab, reslizumab, rovelizumab, siplizumab, talizumab, telimomab aritox, vapaliximab, vepalimomab, abatacept, belatacept, etanercept, pegsunercept, aflibercept, alefacept and rilonacept, and small molecules targeting the ubiquitous JAK or S1P pathways.

Examples of cytokines include, without limitation, chemokines (such as, e.g., CCL1, CCL2/MCP1, CCL3/MIP1α, CCL4/MIP1B, CCL5/RANTES, CCL6, CCL7, CCL8, CCL9, CCL11, CCL12, CCL13, CCL14, CCL15, CCL16, CCL17, CCL18/PARC/DCCK1/AMAC1/MIP4, CCL19, CCL20, CCL21, CCL22, CCL23, CCL24, CCL25, CCL26, CCL27, CCL28, CXCL1/KC, CXCL2, CXCL3, CXCL4, CXCL5, CXCL6, CXCL7, CXCL8/IL8, CXCL9, CXCL10, CXCL11, CXCL12, CXCL13, CXCL14, CXCL15, CXCL16, CXCL17, CX3CL1, XCL1 and XCL2), tumor necrosis factors (such as, e.g., TNFA, Lymphotoxin, TNFSF4, TNFSF5/CD40LG, TNFSF6, TNFSF7, TNFSF8, TNFSF9, TNFSF10, TNFSF11, TNFSF13, TNFSF13B and EDA) and interleukins (such as, e.g., IL-1a, IL-1B, IL-1Ra, IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-36Ra, IL-37, IL-38, IFNα, IFNβ, IFNκ, IFNω and GM-CSF).

Examples of immune modulators include, without limitation, filgrastim, pegfilgrastim, lenograstim, molgramostim, sargramostim, ancestim, albinterferon, interferon alfa, peginterferon alfa, interferon beta, peginterferon beta, interferon gamma, aldesleukin, oprelvekin, growth hormone, immunocyanin, pegademase, prolactin, tasonermin, histamine dihydrochloride, poly ICLC, vitamin D, lentinan, plerixafor, roquinimex, mifamurtide, glatiramer acetate, thymopentin, thymosin α1, thymulin, polyinosinic: polycytidylic acid, pidotimod, Bacillus Calmette-Guérin vaccine, melanoma vaccine and sipuleucel-T vaccine.

Other therapeutic drugs suitable for co-administration with the isolated antibody, or binding fragment thereof, nucleic acid, expression vector, composition, pharmaceutical composition or medicament according to the present invention include anti-cancer drugs. For instance, the therapeutic drug suitable for co-administration may be a cytotoxin, a chemotherapeutic agent, a lytic peptide or a radioisotope.

Examples of cytotoxins include, without limitation, radionuclides (e.g., ³⁵S, ¹⁴C, ³²P, ¹²⁵I, ¹³¹I, ⁹⁰Y, ⁸⁹Zr, ²⁰¹Tl, ¹⁸⁶Re, ¹⁸⁸Re, ⁵⁷Cu, ²¹³Bi, and ²¹¹At), conjugated radionuclides, and chemotherapeutic agents. Further examples of cytotoxins include, but are not limited to, antimetabolites (e.g., 5-fluorouricil (5-FU), methotrexate (MTX), fludarabine, etc.), anti-microtubule agents (e.g., vincristine, vinblastine, colchicine, taxanes (such as paclitaxel and docetaxel), etc.), alkylating agents (e.g., cyclophasphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, tenoposide, and camptothecins), or other cytotoxic agents such as ricin, diptheria toxin (DT), Pseudomonas exotoxin (PE) A, PE40, abrin, saporin, pokeweed viral protein, ethidium bromide, glucocorticoid, anthrax toxin and others.

Examples of chemotherapeutic agents include, without limitation, platinum coordination compounds (such as, e.g., cisplatin, carboplatin or oxalyplatin); taxane compounds (such as, e.g., paclitaxel or docetaxel); topoisomerase I inhibitors (such as, e.g., irinotecan or topotecan); topoisomerase II inhibitors (such as, e.g., etoposide or teniposide); vinca alkaloids (such as, e.g., vinblastine, vincristine or vinorelbine); anti-tumor nucleoside derivatives (such as, e.g., 5-fluorouracil, gemcitabine or capecitabine); alkylating agents (such as, e.g., nitrogen mustard or nitrosourea, cyclophosphamide, chlorambucil, carmustine or lomustine; anti-tumor anthracycline derivatives (suc has, e.g., daunorubicin, doxorubicin, idarubicin or mitoxantrone); anti-HER2 antibodies (such as, e.g., trastuzumab); estrogen receptor antagonists or selective estrogen receptor modulators (such as, e.g., tamoxifen, toremifene, droloxifene, faslodex or raloxifene); aromatase inhibitors (such as, e.g., exemestane, anastrozole, letrazole or vorozole); differentiating agents (such as, e.g., retinoids, vitamin D and retinoic acid metabolism blocking agents [RAMBA] such as accutane); DNA methyl transferase inhibitors (such as, e.g., azacytidine); kinase inhibitors (such as, e.g., flavoperidol, imatinib mesylate or gefitinib); farnesyltransferase inhibitors; and HDAC inhibitors.

Examples of lytic peptides include, without limitation, toxins (such as, e.g., Diptheria toxin or Pseudomonas exotoxin).

Examples of radioisotopes include, without limitation, the radionuclides of technetium (e.g., Tc-99 and Tc-97), potassium (e.g., K-40), rubidium (e.g., Rb-82), iodine (e.g., I-123, I-124, I-125, I-129, I-131), cesium (e.g., Cs-135, Cs-137), cobalt (e.g., Co-60), palladium (e.g., Pd-103, Pd-107), cadmium (e.g., Cd-113), strontium (e.g., Sr-89, Sr-90), europium (e.g., Eu-55), tin (e.g., Sn-121, Sn-126), phosphorus (e.g., P-32, P-33), thallium (e.g., Tl-201), indium (e.g., In-111), gallium (e.g., Ga-67, Ga-68), yttrium (e.g., Y-90), iridium (e.g., Ir-192), bismuth (e.g., Bi-213), radium (e.g., Ra-223, Ra-225), and ruthenium (e.g., Ru-106).

The invention further relates to the use of the isolated antibody, or binding fragment thereof, according to the present invention, for detecting or quantifying AGR2 expression in a biological sample; and to methods for detecting or quantifying AGR2 expression in a biological sample comprising contacting said biological sample with the isolated antibody, or binding fragment thereof, of the present invention.

As used herein, a “biological sample” may for instance be a cell, tissue or organ. Examples of biological samples include, but are not limited to, bodily fluids (preferably blood, more preferably blood serum), plasma, urine, feces, synovial fluid, bronchoalveolar lavage fluid, sputum, lymph, ascitic fluids, urine, amniotic fluid, peritoneal fluid, cerebrospinal fluid, pleural fluid, pericardial fluid, and alveolar macrophages, tissue lysates, biopsies and extracts prepared from diseased tissues.

In some embodiments, the uses and methods for detecting or quantifying AGR2 may be in vitro or in vivo.

As used herein, the term “expression” may refer alternatively to the translation of AGR2 (i.e., expression of the protein), to the presence of AGR2 protein within a cell, at a cell surface, or to the presence of secreted or circulating AGR2 protein.

Assays suitable for detecting or quantifying a protein level in a sample are well-known in the art. Examples of such assays include, but are not limited to, mass spectrometry, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA), flow cytometry (FACS), immunofluorescence, immunoprecipitation, and the like.

In some embodiments, determining the expression level of AGR2 specifically corresponds to the detection and quantification of AGR2 protein within a cell. Methods for analyzing the presence of a protein in a cell are well-known to the skilled artisan and include, without limitation, FACS analysis, immunohistochemistry, mass spectrometry, western blot associated with cell fractionation, enzyme-linked immunosorbent assay (ELISA), sandwich ELISA, fluorescent-linked immunosorbent assay (FLISA), enzyme immunoassay (EIA), radioimmunoassay (RIA) or image analysis, for example high content analysis and the like.

In some embodiments, the isolated antibody, or binding fragment thereof, according to the present invention is labeled, as described hereinabove, for detection or diagnostic purposes, or for patient monitoring purposes.

In some embodiments, the isolated antibody, or binding fragment thereof, according to the present invention is used in combination with one or several other antibodies. For instance, other anti-AGR2 antibodies may be used as a capture antibody, while the isolated antibody, or binding fragment thereof, according to the present invention may be used as a detection antibody, or vice versa. A non-limiting example of anti-AGR2 antibody includes, e.g., the antibody provided by Abcam under reference Ab244826.

In some embodiments, the sample is taken or retrieved from a subject prior to any analysis. Accordingly, in this embodiment, the uses and methods for detecting or quantifying AGR2 are in vitro uses and methods.

The invention further relates to the use of the isolated antibody, or binding fragment thereof, of the present invention, for diagnosing or monitoring an AGR2-related disease in a subject, and to methods of diagnosing or monitoring an AGR2-related disease in a subject, comprising contacting a sample from said subject with the isolated antibody, or binding fragment thereof, of the present invention.

The invention further relates to the use of the isolated antibody, or binding fragment thereof, of the present invention, for selecting a subject suffering from an AGR2-related disease for treatment targeting said disease, and to methods of selecting a subject suffering from an AGR2-related disease for treatment targeting said disease, comprising contacting a sample from said subject with the isolated antibody, or binding fragment thereof, of the present invention.

The invention also relates to the use of the isolated antibody, or binding fragment thereof, of the present invention, for monitoring the response of a subject to a treatment targeting an AGR2-related disease, and to methods of monitoring the response of a subject to a treatment targeting an AGR2-related disease, comprising contacting a sample from said subject with the isolated antibody, or binding fragment thereof, of the present invention.

In some embodiments, the isolated antibody, or binding fragment thereof, of the present invention may be used within a companion diagnostic test. For instance, the isolated antibody, or binding fragment thereof, of the present invention may be used within a companion diagnostic test in association with a therapy targeting an AGR2-related disease.

As used herein the term “AGR2-related disease” refers to any disease in which AGR2 is involved. For instance, an “AGR2-related disease” may be a disease in which

AGR2 is dysregulated, for instance overexpressed (up-regulated) or down-regulated, preferably overexpressed. An “AGR2-related disease” may also be a disease in which abnormal extracellular (membrane-bound or secreted) AGR2 can be detected. An “AGR2-related disease” may be a disease in which AGR2 monomer or dimer can be detected.

In particular, an “AGR2-related disease” may for instance be a mucosal inflammatory disease including, but not limited to, Crohn's disease, inflammatory bowel disease and ulcerative colitis disease.

The uses and methods for diagnosing or monitoring an AGR2-related disease may be in vitro or in vivo, preferably in vitro. In some embodiments, the diagnosis method of the invention is an in vitro diagnosis method, i.e., the method of the invention is performed on a biological sample that was obtained from a patient prior to the implementation of the method of the invention. Consequently, in some embodiments, the method of the invention does not comprise obtaining a sample from the patient, i.e., the method of the invention is non-invasive.

In some embodiments, the AGR2-related diseases are AGR2^{high}-related diseases. According to this embodiment, the subject may be diagnosed as affected with or suffering from a AGR2-related disease if AGR2 is detected in a sample from said subject at a level, amount or concentration higher than in a reference subject (e.g., in a substantially healthy subject, or in a subject who is known not to be affected with or suffering from a AGR2-related disease).

The expression “monitoring the response of a subject to a treatment targeting an AGR2-related disease” may for instance mean adapting the treatment. Preferably, “monitoring the response of a subject to a treatment targeting an AGR2-related disease” means changing the drug used to treat the subject, or increasing or reducing the dose, the administration frequency, or changing the administration route of the treatment.

When the method is used to monitor the progression of an AGR2-related disease or to monitor the response of a subject to a treatment, it is repeated at least at two different points in time (e.g., before and after onset of a treatment).

In some embodiments, the uses and methods for diagnosing or monitoring an AGR2-related disease further comprise a step of treating the subject if said subject is diagnosed as being affected with or suffering from a AGR2-related disease.

In some embodiments, the method of selecting a subject suffering from an AGR2-related disease for treatment targeting said disease, further comprises a step of submitting the subject to a treatment targeting said AGR2-related disease, if said subject is selected to undergo treatment targeting said disease.

### BRIEF DESCRIPTION OF THE DRAWINGS

FIG. 1 is a schematic of the 3-dimensional structure of the human AGR2 protein showing in dotted line circles the location of the epitope bound by Agtuzumab and of the epitope bound by TH-106 humanized monoclonal antibody.

FIG. 2 is a schematic of the structure of the human AGR2 protein. Human AGR2 protein comprises from N-terminus to C-terminus: a cleavable signal peptide, an adhesion domain, a dimerization motif, a pseudo-thioredoxin motif, a peptide binding loop, and an endoplasmic reticulum retention signal.

FIGS. 3A-F is a set of graphs showing the cross-reactivity and specificity of Agtuzumab IgG1-LALA and of the humanized anti-AGR2 monoclonal antibodies of the invention (i.e., TH-101 IgG1-LALA, TH-104 IgG1-LALA, TH-106 IgG1-LALA, TH-109 IgG1-LALA, TH-115 IgG1-LALA). Binding capacities of the antibodies towards human AGR2 WT, mouse AGR2, dog AGR2, macaque AGR2, human AGR2 E60A (i.e., monomeric AGR2), human AGR3 and BSA were evaluated by ELISA. Antibodies were tested at an initial concentration of 10 μg/mL and serially diluted ¼ as low as 0.15 ng/mL. FIG. 3A shows Agtuzumab IgG1-LALA.

FIG. 3B shows TH-101 IgG1-LALA humanized monoclonal antibody.

FIG. 3C shows TH-104 IgG1-LALA humanized monoclonal antibody.

FIG. 3D shows TH-106 IgG1-LALA humanized monoclonal antibody.

FIG. 3E shows TH-109 IgG1-LALA humanized monoclonal antibody.

FIG. 3F shows TH-115 IgG1-LALA humanized monoclonal antibody.

FIG. 4 is a graph showing the binding of Agtuzumab IgG1-LALA and of the humanized anti-AGR2 monoclonal antibodies of the invention (i.e., TH-101 IgG1-LALA, TH-104 IgG1-LALA, TH-106 IgG1-LALA, TH-109 IgG1-LALA, TH-115 IgG1-LALA) towards human AGR2 WT evaluated by ELISA. Antibodies were tested at an initial concentration of 10 μg/mL and serially diluted ¼ as low as 0.15 ng/mL.

FIGS. 5A-B is a set of graphs showing the cross-reactivity and specificity of the TH-106 IgG1 and of the TH-106 IgG1-LALA humanized monoclonal antibodies. Binding capacities of the antibodies towards human AGR2 WT, mouse AGR2, dog AGR2, macaque AGR2, human AGR2 E60A (i.e., monomeric AGR2), human AGR3 and BSA were evaluated by ELISA. Antibodies were tested at an initial concentration of 10 μg/mL and serially diluted ¼ as low as 0.15 ng/mL.

FIG. 5A shows TH-106 IgG1 humanized monoclonal antibody.

FIG. 5B shows TH-106 IgG1-LALA humanized monoclonal antibody.

FIGS. 6A-F is a set of histograms showing the parameters measured in a DSS-induced mouse model of acute colitis treated with PBS as a control, or an anti-AGR2 antibody administered intraperitoneally or intravenously at a dose of 5 μg/mouse, 10 μg/mouse, 20 μg/mouse or 40 μg/mouse as indicated. Data are expressed as mean+/−sem. p values were calculated vs DSS+PBS. * p<0.05; ** p<0.01; *** p<0.001.

FIG. 6A shows the disease activity index (DAI) score at euthanasia (day 12).

FIG. 6B shows the stool consistency of feces at sacrifice.

FIG. 6C shows the presence of occult blood in feces.

FIG. 6D shows the ratio of colon weight/size.

FIG. 6E shows global histological score of inflammation.

FIG. 6F shows the myeloperoxidase quantification in the whole colon.

FIGS. 7A-D is a set of graphs showing that blocking eAGR2 using an antibody of the present invention in a DSS-induced mouse model of acute colitis downregulates circulating cytokines and chemokines involved in inflammation (in particular TNFα and IL-6).

FIG. 7A shows the systemic level of TNFα (p<0.0001).

FIG. 7B shows the systemic level of IL-6 (p<0.0001).

FIG. 7C shows the mRNA level of TNFα (p<0.05).

FIG. 7D shows the mRNA level of IL-6 (p<0.05).

FIGS. 8A-C is a set of graphs showing a summary of immunohistochemical results (median±Q) in control mice treated with PBS (CTL), DSS mice treated with PBS (DSS) and DSS mice treated with the anti-AGR2 antibody at a dose of 5 μg or 20 μg for macrophages (F4/80—FIG. 8A), T-cells (CD3—FIG. 8B), and neutrophils (LY6G—FIG. 8C) obtained using digital morphometric analysis. Statistical significance was calculated using Kruskal-Wallis followed by Mann-Whitney U test. * p<0.001.

FIGS. 9A-M is a set of histograms showing the parameters measured in a DSS-induced mouse model of chronic colitis treated with PBS as a control, or an anti-AGR2 antibody administered preventively or curatively intraperitoneally at a dose of 10 μg/mouse. Data are expressed as mean+/−sem. * p<0.05; ** p<0.01; *** p<0.001.

FIG. 9A shows the disease activity index (DAI) score at euthanasia (day 42).

FIG. 9B shows the stool consistency of feces at euthanasia (day 42).

FIG. 9C shows the presence of occult blood in the feces.

FIG. 9D shows the macroscopic score of fibrosis.

FIG. 9E shows the ratio of colon weight/size.

FIG. 9F shows global histological score of inflammation.

FIG. 9G shows the collagen deposition in mice receiving the curative anti-AGR2 antibody.

FIG. 9H shows in mice receiving the preventive anti-AGR2 antibody.

FIG. 9I shows the level of α-SMA mRNA.

FIG. 9J shows the level of fibronectin mRNA.

FIG. 9K shows the level of Col1A mRNA.

FIG. 9L shows the level of TGF-β mRNA.

FIG. 9M shows the quantification of α-SMA staining by immunofluorescence.

TABLE OF SEQUENCES

| SEQ | | |
|---|---|---|
| ID | Sequence | |
| NO | function | Sequence |
| 1 | Human | MEKIPVSAFLLLVALSYTLARDTTVKPGAKKDTKDSRPKLPQTLSRG |
| | AGR2 | WGDQLIWTQTYEEALYKSKTSNKPLMIIHHLDECPHSQALKKVFAEN |
| | protein | KEIQKLAEQFVLLNLVYETTDKHLSPDGQYVPRIMFVDPSLTVRADIT |
| | | GRYSNRLYAYEPADTALLLDNMKKALKLLKTEL |
| 2 | Mouse | MEKFSVSAILLLVAISGTLAKDTTVKSGAKKDPKDSRPKLPQTLSRGW |
| | AGR2 | GDQLIWTQTYEEALYRSKTSNRPLMVIHHLDECPHSQALKKVFAEHK |
| | protein | EIQKLAEQFVLLNLVYETTDKHLSPDGQYVPRIVFVDPSLTVRADITGR |
| | | YSNRLYAYEPSDTALLYDNMKKALKLLKTEL |
| 3 | Rhesus | MEKISVSAFLLLVALSYTLARDTTVKPGAKKDTKDSRPKLPQTLSRG |
| | monkey | WGDQLIWTQTYEEALYKSKTSNKPLMIIHHLDECPHSQALKKVFAEN |
| | AGR2 | KEIQKLAEQFVLLNLVYETTDKHLSPDGQYVPRILFVDPSLTVRADITG |
| | protein | RYSNRLYAYEPADTALLLDNMKKALKLLKTEL |
| 4 | Chimpanzee | MEKISVSAFLLLVALSYTLARDTTVKPGAKKDTKDSRPKLPQTLSRG |
| | AGR2 | WGDQLIWTQTYEEALYKSKTSNKPLMIIHHLDECPHSQALKKVFAEN |
| | protein | KEIQKLAEQFVLLNLVYETTDKHLSPDGQYVPRIMFVDPSLTVRADIT |
| | | GRYSNRLYAYEPADTALLLDNMKKALKLLKTEL |
| 5 | Rat AGR2 | MEKFSVSAILLLVAISGTLAKDTTVKSGSKKDPKDSRPKLPQTLSRGW |
| | protein | GDQLIWTQTYEEALYKSKTSNRPLMVIHHLDECPHSQALKKVFAENK |
| | | EIQKLAEQFVLLNLIYETTDKHLSPDGQYVPRIVFVDPSLTVRADITGR |
| | | YSNRLYAYEPSDTALLHDNMKKALKLLKTEL |
| 6 | Dog AGR2 | MEKISVSAFLLLVALSYTLAKDITVKSGAKKDTKDSAPKLPQTLSRGW |
| | protein | GDQLIWTQTYEEALYKSKTSNKPLMIIHHLDECPHSQALKKVFAENKE |
| | | IQKLAEQFVLLNLVYETTDKHLSPDGQYVPRIIFVDPSLTVRADITGRY |
| | | SNRLYAYEPSDIALLLDNMKKALKLLKTEL |
| 7 | Human | MEKIPVSAFLLLVALSYTLARDTTVKPGAKKDTKDSRPKLPQTLSRG |
| | AGR2 E60A | WGDQLIWTQTYEAALYKSKTSNKPLMIIHHLDECPHSQALKKVFAEN |
| | | KEIQKLAEQFVLLNLVYETTDKHLSPDGQYVPRIMFVDPSLTVRADIT |
| | | GRYSNRLYAYEPADTALLLDNMKKALKLLKTEL |
| 8 | VH-CDR1 | RSWMN |
| 9 | Consensus | WIYPGDGDTNYNGKXKD |
| | VH-CDR2 | wherein X is For V |
| 10 | VH-CDR2 | WIYPGDGDTNYNGKFKD |
| 11 | VH-CDR2 | WIYPGDGDTNYNGKVKD |
| 12 | VH-CDR3 | GGYDGSPWLSY |
| 13 | VL-CDR1 | KASQDINSYLS |
| 14 | VL-CDR2 | RANRLVD |
| 15 | VL-CDR3 | LQYDEFPFT |
| 16 | VH-146 | QVQLVQSGAEVKKPGASVKVSCKTSGYVFSRSWMNWVRQAPGQGLEWI |
| | | GWIYPGDGDTNYNGKFKDRVTLTADKSTSTVYMELSSLRSEDTAVYFCA |
| | | RGGYDGSPWLSYWGQGTLVTVSS |
| 17 | VH-741 | QVQLVQSGSELKKPGASVKVSCKTSGYVFSRSWMNWVRQAPGQGLEW |
| | | IGWIYPGDGDTNYNGKFKDRFVLSADKSVSTVYLQISSLKAEDTAVYF |
| | | CARGGYDGSPWLSYWGQGTLVTVSS |
| 18 | VH-311 | QVQLLESGGGLVKPGGSLRLSCATSGYVFSRSWMNWIRQAPGQGLEWI |
| | | GWIYPGDGDTNYNGKVKDRFTLSADKAKNSVYLQMNSLRAEDTAVY |
| | | FCARGGYDGSPWLSYWGQGTLVTVSS |
| 19 | VL-133 | DIQMTQSPSSLSASVGDRVTITCKASQDINSYLSWFQQKPGKAPKTLIY |
| | | RANRLVDGVPSRFSGSGSGQDFTLTISSLQPEDIATYYCLQYDEFPFTFGG |
| | | GTKVEIK |
| 20 | VL-311 | EIVLTQSPATLSLSPGERATLSCKASQDINSYLSWFQQKPGQAPRTLIY |
| | | RANRLVDGIPARFSGSGSGQDFTLTISSLEPEDFAVYYCLQYDEFPFTFGG |
| | | GTKVEIK |
| 21 | VL-621 | EIVLTQSPDFQSVTPKEKVTITCKASQDINSYLSWFQQKPDQSPKTLIY |
| | | RANRLVDGVPSRFSGSGSGQDFTLTINSLEAEDAATYYCLQYDEFPFTFG |
| | | GGTKVEIK |
| 22 | Heavy chain | ASTKGPSVFPLAPSSKSTSGGTAALGCLVKDYFPEPVTVSWNSGALTSGV |
| | constant | HTFPAVLQSSGLYSLSSVVTVPSSSLGTQTYICNVNHKPSNTKVDKKVEP |
| | region- | KSCDKTHTCPPCPAPELLGGPSVFLFPPKPKDTLMISRTPEVTCVVVDVS |
| | IgG1 | HEDPEVKFNWYVDGVEVHNAKTKPREEQYNSTYRVVSVLTVLHQDWL |
| | | NGKEYKCKVSNKALPAPIEKTISKAKGQPREPQVYTLPPSRDELTKNQVS |
| | | LTCLVKGFYPSDIAVEWESNGQPENNYKTTPPVLDSDGSFFLYSKLTVD |
| | | KSRWQQGNVFSCSVMHEALHNHYTQKSLSLSPGK |
| 23 | Heavy chain | ASTKGPSVFPLAPSSKSTSGGTAALGCLVKDYFPEPVTVSWNSGALTS |
| | constant | GVHTFPAVLQSSGLYSLSSVVTVPSSSLGTQTYICNVNHKPSNTKVDK |
| | region- | KVEPKSCDKTHTCPPCPAPEAAGGPSVFLFPPKPKDTLMISRTPEVTCV |
| | IgG1-LALA | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 24 | Heavy chain | ASTKGPSVFPLAPSSKSTSGGTAALGCLVKDYFPEPVTVSWNSGALTS |
| | constant | GVHTFPAVLQSSGLYSLSSVVTVPSSSLGTQTYICNVNHKPSNTKVDK |
| | region- | KVEPKSCDKTHTCPPCPAPEFEGGPSVFLFPPKPKDTLMISRTPEVTCV |
| | IgG1- | VVDVSHEDPEVKFNWYVDGVEVHNAKTKPREEQYNSTYRVVSVLTV |
| | LFLEPS | LHQDWLNGKEYKCKVSNKALPASIEKTISKAKGQPREPQVYTLPPSRD |
| | | ELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 25 | Heavy chain | ASTKGPSVFPLAPCSRSTSESTAALGCLVKDYFPEPVTVSWNSGALTS |
| | constant | GVHTFPAVLQSSGLYSLSSVVTVPSSSLGTKTYTCNVDHKPSNTKVDK |
| | region- | RVESKYGPPCPSCPAPEFLGGPSVFLFPPKPKDTLMISRTPEVTCVVVD |
| | IgG4 | VSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLY |
| | | SRLTVDKSRWQEGNVFSCSVMHEALHNHYTQKSLSLSLGK |
| 26 | Light chain | RTVAAPSVFIFPPSDEQLKSGTASVVCLLNNFYPREAKVQWKVDNALQS |
| | constant | GNSQESVTEQDSKDSTYSLSSTLTLSKADYEKHKVYACEVTHQGLSSPV |
| | region | TKSFNRGEC |
| 27 | Heavy chain | QVQLVQSGAEVKKPGASVKVSCKTSGYVFSRSWMNWVRQAPGQGLE |
| | 146-IgG1 | WIGWIYPGDGDTNYNGKFKDRVTLTADKSTSTVYMELSSLRSEDTAVY |
| | | FCARGGYDGSPWLSYWGQGTLVTVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 28 | Heavy chain | QVQLVQSGSELKKPGASVKVSCKTSGYVFSRSWMNWVRQAPGQGLEW |
| | 741-IgG1 | IGWIYPGDGDTNYNGKFKDRFVLSADKSVSTVYLQISSLKAEDTAVYFC |
| | | ARGGYDGSPWLSYWGQGTLVTVSSASTKGPSVFPLAPSSKSTSGGTAAL |
| | | GCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPGK |
| 29 | Heavy chain | QVQLLESGGGLVKPGGSLRLSCATSGYVFSRSWMNWIRQAPGQGLEWI |
| | 311-IgG1 | GWIYPGDGDTNYNGKVKDRFTLSADKAKNSVYLQMNSLRAEDTAVYF |
| | | CARGGYDGSPWLSYWGQGTLVTVSSASTKGPSVFPLAPSSKSTSGGTAA |
| | | LGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNH |
| | | YTQKSLSLSPGK |
| 30 | Heavy chain | QVQLVQSGAEVKKPGASVKVSCKTSGYVFSRSWMNWVRQAPGQGLE |
| | 146-IgG1- | WIGWIYPGDGDTNYNGKFKDRVTLTADKSTSTVYMELSSLRSEDTAVY |
| | LALA | FCARGGYDGSPWLSYWGQGTLVTVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 31 | Heavy chain | QVQLVQSGSELKKPGASVKVSCKTSGYVFSRSWMNWVRQAPGQGLEW |
| | 741-IgG1- | IGWIYPGDGDTNYNGKFKDRFVLSADKSVSTVYLQISSLKAEDTAVYFC |
| | LALA | ARGGYDGSPWLSYWGQGTLVTVSSASTKGPSVFPLAPSSKSTSGGTAAL |
| | | GCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNH |
| | | YTQKSLSLSPGK |
| 32 | Heavy chain | QVQLLESGGGLVKPGGSLRLSCATSGYVFSRSWMNWIRQAPGQGLEWI |
| | 311-IgG1- | GWIYPGDGDTNYNGKVKDRFTLSADKAKNSVYLQMNSLRAEDTAVYF |
| | LALA | CARGGYDGSPWLSYWGQGTLVTVSSASTKGPSVFPLAPSSKSTSGGTAA |
| | | LGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHN |
| | | HYTQKSLSLSPGK |
| 33 | Light chain | DIQMTQSPSSLSASVGDRVTITCKASQDINSYLSWFQQKPGKAPKTLIYR |
| | 133 | ANRLVDGVPSRFSGSGSGQDFTLTISSLQPEDIATYYCLQYDEFPFTFGGG |
| | | TKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQ |
| | | GLSSPVTKSFNRGEC |
| 34 | Light chain | EIVLTQSPATLSLSPGERATLSCKASQDINSYLSWFQQKPGQAPRTLIYRA |
| | 311 | NRLVDGIPARFSGSGSGQDFTLTISSLEPEDFAVYYCLQYDEFPFTFGGGT |
| | | KVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| 35 | Light chain | EIVLTQSPDFQSVTPKEKVTITCKASQDINSYLSWFQQKPDQSPKTLIYRA |
| | 621 | NRLVDGVPSRFSGSGSGQDFTLTINSLEAEDAATYYCLQYDEFPFTFGGG |
| | | TKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQ |
| | | GLSSPVTKSFNRGEC |
| 36 | TH-101 | ATGGGCTGGTCCTGCATCATCCTGTTCCTGGTGGCTACCGCCACCG |
| | heavy chain | GCGTGCACTCCCAAGTGCAACTAGTGCAAAGTGGTGCAGAAGTCA |
| | IgG1-LALA | AGAAGCCTGGCGCTTCCGTGAAAGTGTCCTGCAAGACCAGCGGAT |
| | nucleic acid | ACGTGTTCTCCCGGTCCTGGATGAACTGGGTGCGGCAGGCTCCTGG |
| | | ACAGGGCCTGGAGTGGATCGGCTGGATCTACCCCGGCGATGGCGA |
| | | CACCAACTACAACGGCAAGTTCAAGGACAGAGTGACCCTGACCGC |
| | | CGACAAGTCCACCTCTACCGTGTACATGGAACTGTCTTCTCTGAGA |
| | | TCCGAGGATACAGCCGTGTATTTTTGTGCCAGAGGCGGCTACGACG |
| | | GCTCTCCTTGGCTGTCCTACTGGGGCCAGGGCACACTGGTGACCGT |
| | | GTCTAGCGCAAGTACTAAGGGTCCAAGTGTGTTTCCACTGGCTCCC |
| | | TCTAGCAAGTCCACCTCTGGCGGCACCGCCGCCCTGGGCTGCCTGG |
| | | TCAAGGACTACTTCCCCGAGCCCGTCACCGTGTCCTGGAACTCCGG |
| | | CGCTCTGACCTCCGGCGTGCATACCTTCCCTGCTGTGCTGCAGTCTT |
| | | CTGGCCTGTACTCCCTGTCCTCCGTCGTGACAGTGCCTTCTAGCTCT |
| | | CTCGGCACACAGACATACATCTGCAATGTGAACCACAAGCCTTCCA |
| | | ACACCAAAGTGGATAAGAAGGTGGAACCTAAGTCCTGCGACAAAA |
| | | CCCACACCTGTCCTCCTTGCCCTGCTCCTGAAGCCGCTGGCGGACC |
| | | TTCTGTGTTTCTGTTTCCCCCAAAGCCCAAAGACACCCTGATGATCT |
| | | CTCGGACACCTGAGGTGACCTGCGTGGTGGTGGATGTGTCTCACGA |
| | | GGACCCCGAAGTGAAGTTCAACTGGTACGTGGACGGCGTGGAAGT |
| | | GCACAACGCCAAGACCAAGCCACGCGAAGAGCAGTACAACTCTAC |
| | | CTACAGAGTGGTGTCCGTGCTCACAGTGCTGCACCAGGACTGGCTG |
| | | AACGGAAAAGAGTACAAGTGCAAGGTCTCTAACAAGGCTCTGCCA |
| | | GCCCCTATCGAGAAGACCATCTCCAAGGCCAAGGGCCAACCTAGA |
| | | GAGCCTCAAGTGTACACCCTGCCTCCTAGCCGGGACGAGCTGACCA |
| | | AGAACCAGGTTTCCCTGACCTGTCTGGTGAAAGGCTTCTACCCCTC |
| | | CGATATCGCCGTGGAATGGGAGTCCAACGGCCAGCCTGAGAACAA |
| | | CTACAAGACCACCCCTCCTGTGCTGGACTCCGATGGATCCTTCTTC |
| | | CTGTATAGCAAGCTGACCGTGGACAAGTCCAGATGGCAGCAGGGC |
| | | AACGTGTTCAGCTGTTCCGTGATGCACGAGGCCCTGCATAATCACT |
| | | ACACCCAGAAGTCTCTGAGCCTGTCTCCTGGCAAGTGA |
| 37 | TH-101 light | ATGGGCTGGTCCTGCATCATCCTGTTCCTGGTGGCTACCGCCACCG |
| | chain IgG1- | GCGTGCACTCCGATATCCAAATGACTCAAAGTCCAAGTAGTCTCTC |
| | LALA | TGCCTCCGTGGGCGACAGAGTGACAATCACCTGTAAGGCCTCCCAA |
| | nucleic acid | GATATCAACTCCTATCTGTCCTGGTTCCAGCAGAAGCCTGGCAAGG |
| | | CTCCTAAGACCCTGATCTACAGAGCCAACCGGCTGGTCGATGGCGT |
| | | GCCCAGCCGGTTCTCCGGCTCTGGATCTGGCCAGGACTTCACACTG |
| | | ACCATCTCCAGCCTGCAGCCTGAGGACATCGCCACCTACTACTGCC |
| | | TGCAGTACGACGAGTTCCCATTTACCTTCGGCGGCGGCACCAAGGT |
| | | GGAAATCAAGAGAACCGTGGCCGCTCCTTCTGTGTTCATCTTCCCT |
| | | CCTTCCGACGAGCAGCTGAAGTCCGGCACCGCTTCCGTGGTGTGCC |
| | | TGCTGAACAACTTCTACCCTAGAGAGGCCAAGGTGCAGTGGAAGG |
| | | TGGACAACGCCCTGCAGTCTGGCAACTCCCAAGAGTCCGTCACCGA |
| | | GCAGGACTCCAAGGACTCTACCTACTCCCTGTCCTCCACCCTGACA |
| | | CTGAGCAAGGCCGACTACGAGAAGCACAAGGTGTACGCCTGCGAG |
| | | GTGACCCACCAGGGCCTGTCTTCTCCTGTGACCAAGTCCTTCAACA |
| | | GAGGCGAGTGCTGA |
| 38 | TH-104 | ATGGGCTGGTCCTGCATCATCCTGTTCCTGGTGGCTACCGCCACCG |
| | heavy chain | GCGTGCACTCCCAAGTGCAACTAGTGCAAAGTGGTAGTGAACTGA |
| | IgG1-LALA | AAAAGCCTGGAGCTTCTGTGAAGGTGTCCTGCAAGACATCTGGCTA |
| | nucleic acid | CGTGTTCTCCAGATCTTGGATGAACTGGGTGCGGCAGGCTCCTGGC |
| | | CAGGGCCTGGAATGGATCGGCTGGATCTACCCCGGAGATGGCGAT |
| | | ACCAACTACAACGGCAAGTTCAAGGACCGGTTCGTGCTGTCTGCCG |
| | | ACAAGTCCGTGTCCACCGTGTACCTGCAGATCTCCTCCCTCAAGGC |
| | | CGAGGACACCGCCGTGTATTTTTGTGCCAGAGGCGGCTACGACGGC |
| | | TCTCCTTGGCTGTCCTACTGGGGCCAAGGCACCCTGGTGACCGTCA |
| | | GCAGCGCAAGTACTAAGGGTCCAAGTGTGTTTCCACTGGCTCCCTC |
| | | TAGCAAGTCCACCTCTGGCGGCACCGCCGCCCTGGGCTGCCTGGTC |
| | | AAGGACTACTTCCCCGAGCCCGTCACCGTGTCCTGGAACTCCGGCG |
| | | CTCTGACCTCCGGCGTGCATACCTTCCCTGCTGTGCTGCAGTCTTCT |
| | | GGCCTGTACTCCCTGTCCTCCGTCGTGACAGTGCCTTCTAGCTCTCT |
| | | CGGCACACAGACATACATCTGCAATGTGAACCACAAGCCTTCCAA |
| | | CACCAAAGTGGATAAGAAGGTGGAACCTAAGTCCTGCGACAAAAC |
| | | CCACACCTGTCCTCCTTGCCCTGCTCCTGAAGCCGCTGGCGGACCT |
| | | TCTGTGTTTCTGTTTCCCCCAAAGCCCAAAGACACCCTGATGATCTC |
| | | TCGGACACCTGAGGTGACCTGCGTGGTGGTGGATGTGTCTCACGAG |
| | | GACCCCGAAGTGAAGTTCAACTGGTACGTGGACGGCGTGGAAGTG |
| | | CACAACGCCAAGACCAAGCCACGCGAAGAGCAGTACAACTCTACC |
| | | TACAGAGTGGTGTCCGTGCTCACAGTGCTGCACCAGGACTGGCTGA |
| | | ACGGAAAAGAGTACAAGTGCAAGGTCTCTAACAAGGCTCTGCCAG |
| | | CCCCTATCGAGAAGACCATCTCCAAGGCCAAGGGCCAACCTAGAG |
| | | AGCCTCAAGTGTACACCCTGCCTCCTAGCCGGGACGAGCTGACCAA |
| | | GAACCAGGTTTCCCTGACCTGTCTGGTGAAAGGCTTCTACCCCTCC |
| | | GATATCGCCGTGGAATGGGAGTCCAACGGCCAGCCTGAGAACAAC |
| | | TACAAGACCACCCCTCCTGTGCTGGACTCCGATGGATCCTTCTTCCT |
| | | GTATAGCAAGCTGACCGTGGACAAGTCCAGATGGCAGCAGGGCAA |
| | | CGTGTTCAGCTGTTCCGTGATGCACGAGGCCCTGCATAATCACTAC |
| | | ACCCAGAAGTCTCTGAGCCTGTCTCCTGGCAAGTGA |
| 39 | TH-104 light | ATGGGCTGGTCCTGCATCATCCTGTTCCTGGTGGCTACCGCCACCG |
| | chain IgG1- | GCGTGCACTCCGATATCCAAATGACTCAAAGTCCAAGTAGTCTGTC |
| | LALA | TGCCAGCGTCGGCGATAGAGTGACAATCACCTGTAAGGCCTCCCA |
| | nucleic acid | AGATATCAACTCCTATCTGTCCTGGTTCCAGCAGAAGCCTGGCAAG |
| | | GCTCCTAAGACCCTGATCTACAGAGCCAACCGGCTGGTGGACGGC |
| | | GTGCCATCTCGGTTCTCCGGATCTGGCTCCGGCCAGGACTTCACAC |
| | | TGACCATCTCCAGCCTGCAGCCTGAGGACATCGCCACCTACTACTG |
| | | CCTGCAGTACGACGAGTTCCCCTTTACCTTCGGCGGAGGCACCAAG |
| | | GTGGAAATCAAGAGAACCGTGGCCGCTCCTTCTGTGTTCATCTTCC |
| | | CTCCTTCCGACGAGCAGCTGAAGTCCGGCACCGCTTCCGTGGTGTG |
| | | CCTGCTGAACAACTTCTACCCTAGAGAGGCCAAGGTGCAGTGGAA |
| | | GGTGGACAACGCCCTGCAGTCTGGCAACTCCCAAGAGTCCGTCACC |
| | | GAGCAGGACTCCAAGGACTCTACCTACTCCCTGTCCTCCACCCTGA |
| | | CACTGAGCAAGGCCGACTACGAGAAGCACAAGGTGTACGCCTGCG |
| | | AGGTGACCCACCAGGGCCTGTCTTCTCCTGTGACCAAGTCCTTCAA |
| | | CAGAGGCGAGTGCTGA |
| 40 | TH-106 | ATGGGCTGGTCCTGCATCATCCTGTTCCTGGTGGCTACCGCCACCG |
| | heavy chain | GCGTGCACTCCCAAGTGCAACTAGTGCAAAGTGGTGCAGAAGTCA |
| | IgG1-LALA | AGAAGCCTGGCGCTTCCGTGAAAGTGTCCTGCAAGACCTCTGGCTA |
| | nucleic acid | CGTGTTCTCCCGGTCCTGGATGAACTGGGTGCGGCAGGCCCCTGGA |
| | | CAGGGCCTGGAGTGGATCGGCTGGATCTACCCCGGCGATGGCGAC |
| | | ACCAACTACAACGGCAAGTTCAAGGACAGAGTGACACTGACCGCC |
| | | GACAAGTCCACATCTACCGTGTACATGGAACTGTCTAGCCTGAGAT |
| | | CCGAGGATACCGCTGTGTATTTTTGTGCCAGAGGCGGATACGACGG |
| | | CTCTCCTTGGCTGTCCTACTGGGGCCAGGGCACCCTGGTGACCGTG |
| | | TCTAGCGCAAGTACTAAGGGTCCAAGTGTGTTTCCACTGGCTCCCT |
| | | CTAGCAAGTCCACCTCTGGCGGCACCGCCGCCCTGGGCTGCCTGGT |
| | | CAAGGACTACTTCCCCGAGCCCGTCACCGTGTCCTGGAACTCCGGC |
| | | GCTCTGACCTCCGGCGTGCATACCTTCCCTGCTGTGCTGCAGTCTTC |
| | | TGGCCTGTACTCCCTGTCCTCCGTCGTGACAGTGCCTTCTAGCTCTC |
| | | TCGGCACACAGACATACATCTGCAATGTGAACCACAAGCCTTCCAA |
| | | CACCAAAGTGGATAAGAAGGTGGAACCTAAGTCCTGCGACAAAAC |
| | | CCACACCTGTCCTCCTTGCCCTGCTCCTGAAGCCGCTGGCGGACCT |
| | | TCTGTGTTTCTGTTTCCCCCAAAGCCCAAAGACACCCTGATGATCTC |
| | | TCGGACACCTGAGGTGACCTGCGTGGTGGTGGATGTGTCTCACGAG |
| | | GACCCCGAAGTGAAGTTCAACTGGTACGTGGACGGCGTGGAAGTG |
| | | CACAACGCCAAGACCAAGCCACGCGAAGAGCAGTACAACTCTACC |
| | | TACAGAGTGGTGTCCGTGCTCACAGTGCTGCACCAGGACTGGCTGA |
| | | ACGGAAAAGAGTACAAGTGCAAGGTCTCTAACAAGGCTCTGCCAG |
| | | CCCCTATCGAGAAGACCATCTCCAAGGCCAAGGGCCAACCTAGAG |
| | | AGCCTCAAGTGTACACCCTGCCTCCTAGCCGGGACGAGCTGACCAA |
| | | GAACCAGGTTTCCCTGACCTGTCTGGTGAAAGGCTTCTACCCCTCC |
| | | GATATCGCCGTGGAATGGGAGTCCAACGGCCAGCCTGAGAACAAC |
| | | TACAAGACCACCCCTCCTGTGCTGGACTCCGATGGATCCTTCTTCCT |
| | | GTATAGCAAGCTGACCGTGGACAAGTCCAGATGGCAGCAGGGCAA |
| | | CGTGTTCAGCTGTTCCGTGATGCACGAGGCCCTGCATAATCACTAC |
| | | ACCCAGAAGTCTCTGAGCCTGTCTCCTGGCAAGTGA |
| 41 | TH-106 light | ATGGGCTGGTCCTGCATCATCCTGTTCCTGGTGGCTACCGCCACCG |
| | chain IgG1- | GCGTGCACTCCGAAATCGTGCTAACTCAAAGTCCAGCAACTCTCAG |
| | LALA | CCTGTCTCCAGGCGAGCGGGCTACACTGAGCTGCAAGGCCTCTCAG |
| | nucleic acid | GACATCAACTCCTACCTGTCCTGGTTCCAGCAGAAACCTGGCCAGG |
| | | CTCCTAGAACCCTGATCTACAGAGCCAACCGGCTGGTGGACGGCAT |
| | | CCCTGCCAGATTCTCCGGCTCTGGATCTGGCCAAGATTTTACCCTG |
| | | ACCATCTCCTCCCTGGAACCTGAGGACTTCGCCGTGTACTATTGTCT |
| | | GCAGTACGACGAGTTCCCCTTCACCTTCGGCGGAGGCACCAAGGTG |
| | | GAAATCAAGAGAACCGTGGCCGCTCCTTCTGTGTTCATCTTCCCTC |
| | | CTTCCGACGAGCAGCTGAAGTCCGGCACCGCTTCCGTGGTGTGCCT |
| | | GCTGAACAACTTCTACCCTAGAGAGGCCAAGGTGCAGTGGAAGGT |
| | | GGACAACGCCCTGCAGTCTGGCAACTCCCAAGAGTCCGTCACCGA |
| | | GCAGGACTCCAAGGACTCTACCTACTCCCTGTCCTCCACCCTGACA |
| | | CTGAGCAAGGCCGACTACGAGAAGCACAAGGTGTACGCCTGCGAG |
| | | GTGACCCACCAGGGCCTGTCTTCTCCTGTGACCAAGTCCTTCAACA |
| | | GAGGCGAGTGCTGA |
| 42 | TH-106 | ATGGGCTGGTCCTGCATCATCCTGTTTCTGGTGGCCACCGCTACCG |
| | heavy chain | GAGTGCACTCCCAAGTGCAACTAGTGCAAAGTGGTGCAGAAGTCA |
| | IgG1 nucleic | AGAAGCCTGGCGCTTCCGTGAAAGTGTCCTGCAAGACCTCCGGCTA |
| | acid | CGTGTTCTCCCGGTCTTGGATGAACTGGGTGCGGCAGGCCCCTGGA |
| | | CAGGGCCTGGAGTGGATCGGCTGGATCTACCCCGGAGATGGCGAT |
| | | ACCAACTACAACGGCAAGTTCAAGGACAGAGTGACACTGACCGCT |
| | | GACAAGTCCACCTCTACCGTGTACATGGAACTGTCTAGCCTGAGAT |
| | | CCGAGGACACCGCCGTGTATTTTTGTGCCAGAGGCGGCTACGACGG |
| | | CTCTCCTTGGCTGTCCTACTGGGGCCAGGGCACCCTGGTGACAGTG |
| | | TCTAGCGCCTCTACAAAGGGCCCATCTGTGTTTCCTCTGGCCCCTTC |
| | | TTCTAAGTCTACCTCCGGCGGAACAGCCGCTCTGGGCTGTCTGGTC |
| | | AAGGACTACTTCCCTGAGCCCGTGACCGTGTCCTGGAACTCTGGCG |
| | | CTCTGACCTCTGGAGTGCATACCTTCCCCGCTGTGCTGCAGTCCTCT |
| | | GGCCTGTACTCTCTGTCTAGCGTCGTGACCGTGCCTTCTTCCAGCCT |
| | | GGGCACCCAGACCTACATCTGCAACGTGAACCACAAGCCTTCCAA |
| | | CACCAAAGTGGATAAGAAGGTGGAACCTAAGAGCTGCGACAAGAC |
| | | ACACACCTGCCCTCCTTGTCCTGCTCCTGAACTGCTGGGCGGCCCT |
| | | AGCGTGTTCCTGTTCCCACCTAAGCCCAAGGACACCCTGATGATCT |
| | | CTCGGACCCCTGAGGTCACCTGTGTGGTGGTGGACGTGTCTCACGA |
| | | GGACCCCGAGGTGAAGTTCAACTGGTACGTGGATGGCGTGGAAGT |
| | | GCACAACGCCAAGACCAAGCCTCGGGAAGAGCAGTACAACTCCAC |
| | | CTACAGAGTGGTCTCTGTCCTGACCGTGCTGCACCAGGACTGGCTG |
| | | AACGGCAAAGAGTACAAGTGCAAGGTGTCCAACAAGGCCCTGCCT |
| | | GCCCCTATCGAGAAGACCATCTCCAAGGCTAAGGGCCAGCCTAGA |
| | | GAGCCTCAAGTGTACACCCTGCCTCCAAGCCGGGACGAGCTGACC |
| | | AAGAACCAGGTGTCTCTGACCTGCCTGGTGAAAGGCTTCTACCCCT |
| | | CCGACATCGCCGTGGAATGGGAGTCCAATGGCCAACCCGAGAACA |
| | | ACTACAAGACCACCCCTCCTGTGCTGGACTCCGACGGCTCCTTCTT |
| | | CCTGTACTCCAAGCTGACAGTGGACAAGTCCAGATGGCAGCAGGG |
| | | CAACGTGTTCTCCTGCTCCGTGATGCACGAGGCCCTGCACAATCAC |
| | | TACACCCAGAAGTCCCTGTCCCTCTCTCCTGGCAAGTGA |
| 43 | TH-106 light | ATGGGCTGGTCCTGCATCATCCTGTTCCTGGTGGCTACCGCCACCG |
| | chain IgG1 | GCGTGCACTCCGAAATCGTGCTAACTCAAAGTCCAGCAACTCTCTC |
| | nucleic acid | TCTGTCTCCTGGCGAGCGGGCTACCCTGTCCTGCAAGGCCTCTCAG |
| | | GACATCAACTCCTACCTGTCCTGGTTCCAGCAGAAACCAGGACAAG |
| | | CTCCTAGAACCCTGATCTACAGAGCCAACCGGCTGGTGGACGGCAT |
| | | CCCTGCCAGATTCTCCGGCAGCGGCTCTGGCCAGGATTTTACACTG |
| | | ACCATCTCCAGCCTGGAACCTGAGGACTTCGCCGTGTACTATTGTC |
| | | TGCAGTACGACGAGTTCCCCTTCACCTTCGGCGGAGGCACCAAGGT |
| | | GGAAATCAAGAGAACCGTGGCCGCTCCTTCTGTGTTCATCTTCCCT |
| | | CCTTCCGACGAGCAGCTGAAGTCCGGCACCGCTTCCGTGGTGTGCC |
| | | TGCTGAACAACTTCTACCCTAGAGAGGCCAAGGTGCAGTGGAAGG |
| | | TGGACAACGCCCTGCAGTCTGGCAACTCCCAAGAGTCCGTCACCGA |
| | | GCAGGACTCCAAGGACTCTACCTACTCCCTGTCCTCCACCCTGACA |
| | | CTGAGCAAGGCCGACTACGAGAAGCACAAGGTGTACGCCTGCGAG |
| | | GTGACCCACCAGGGCCTGTCTTCTCCTGTGACCAAGTCCTTCAACA |
| | | GAGGCGAGTGCTGA |
| 44 | TH-109 | ATGGGCTGGTCCTGCATCATCCTGTTCCTGGTGGCTACCGCCACCG |
| | heavy chain | GCGTGCACTCCCAAGTGCAACTAGTGCAAAGTGGTAGTGAACTCA |
| | IgG1-LALA | AAAAGCCTGGCGCCTCTGTGAAGGTGTCCTGCAAGACCTCTGGCTA |
| | nucleic acid | CGTGTTCTCCAGATCTTGGATGAACTGGGTGCGGCAGGCTCCTGGA |
| | | CAAGGCCTGGAATGGATCGGCTGGATCTACCCCGGCGATGGAGAT |
| | | ACCAACTACAACGGCAAGTTCAAGGACCGGTTCGTGCTGTCTGCCG |
| | | ACAAGTCCGTGTCCACCGTGTACCTGCAGATCTCCTCCCTGAAGGC |
| | | CGAGGACACAGCTGTCTATTTTTGTGCCAGAGGCGGCTACGACGGC |
| | | TCTCCTTGGCTGTCCTACTGGGGCCAGGGCACCCTGGTGACCGTTA |
| | | GCAGCGCAAGTACTAAGGGTCCAAGTGTGTTTCCACTGGCTCCCTC |
| | | TAGCAAGTCCACCTCTGGCGGCACCGCCGCCCTGGGCTGCCTGGTC |
| | | AAGGACTACTTCCCCGAGCCCGTCACCGTGTCCTGGAACTCCGGCG |
| | | CTCTGACCTCCGGCGTGCATACCTTCCCTGCTGTGCTGCAGTCTTCT |
| | | GGCCTGTACTCCCTGTCCTCCGTCGTGACAGTGCCTTCTAGCTCTCT |
| | | CGGCACACAGACATACATCTGCAATGTGAACCACAAGCCTTCCAA |
| | | CACCAAAGTGGATAAGAAGGTGGAACCTAAGTCCTGCGACAAAAC |
| | | CCACACCTGTCCTCCTTGCCCTGCTCCTGAAGCCGCTGGCGGACCT |
| | | TCTGTGTTTCTGTTTCCCCCAAAGCCCAAAGACACCCTGATGATCTC |
| | | TCGGACACCTGAGGTGACCTGCGTGGTGGTGGATGTGTCTCACGAG |
| | | GACCCCGAAGTGAAGTTCAACTGGTACGTGGACGGCGTGGAAGTG |
| | | CACAACGCCAAGACCAAGCCACGCGAAGAGCAGTACAACTCTACC |
| | | TACAGAGTGGTGTCCGTGCTCACAGTGCTGCACCAGGACTGGCTGA |
| | | ACGGAAAAGAGTACAAGTGCAAGGTCTCTAACAAGGCTCTGCCAG |
| | | CCCCTATCGAGAAGACCATCTCCAAGGCCAAGGGCCAACCTAGAG |
| | | AGCCTCAAGTGTACACCCTGCCTCCTAGCCGGGACGAGCTGACCAA |
| | | GAACCAGGTTTCCCTGACCTGTCTGGTGAAAGGCTTCTACCCCTCC |
| | | GATATCGCCGTGGAATGGGAGTCCAACGGCCAGCCTGAGAACAAC |
| | | TACAAGACCACCCCTCCTGTGCTGGACTCCGATGGATCCTTCTTCCT |
| | | GTATAGCAAGCTGACCGTGGACAAGTCCAGATGGCAGCAGGGCAA |
| | | CGTGTTCAGCTGTTCCGTGATGCACGAGGCCCTGCATAATCACTAC |
| | | ACCCAGAAGTCTCTGAGCCTGTCTCCTGGCAAGTGA |
| 45 | TH-109 light | ATGGGCTGGTCCTGCATCATCCTGTTCCTGGTGGCTACCGCCACCG |
| | chain IgG1- | GCGTGCACTCCGAAATCGTGCTAACTCAAAGTCCAGCAACTCTCAG |
| | LALA | CCTGAGCCCTGGCGAGCGGGCTACCCTGTCCTGCAAGGCCTCTCAG |
| | nucleic acid | GACATCAACTCCTACCTGTCTTGGTTCCAGCAGAAACCAGGCCAGG |
| | | CTCCTAGAACCCTGATCTACAGAGCCAACCGGCTGGTGGACGGCAT |
| | | CCCTGCCAGATTCTCCGGCTCTGGCTCCGGACAAGATTTTACACTG |
| | | ACCATCTCCTCTCTGGAACCTGAGGACTTCGCCGTGTACTATTGTCT |
| | | GCAGTACGACGAGTTCCCCTTCACCTTCGGCGGAGGCACCAAGGTG |
| | | GAAATCAAGAGAACCGTGGCCGCTCCTTCTGTGTTCATCTTCCCTC |
| | | CTTCCGACGAGCAGCTGAAGTCCGGCACCGCTTCCGTGGTGTGCCT |
| | | GCTGAACAACTTCTACCCTAGAGAGGCCAAGGTGCAGTGGAAGGT |
| | | GGACAACGCCCTGCAGTCTGGCAACTCCCAAGAGTCCGTCACCGA |
| | | GCAGGACTCCAAGGACTCTACCTACTCCCTGTCCTCCACCCTGACA |
| | | CTGAGCAAGGCCGACTACGAGAAGCACAAGGTGTACGCCTGCGAG |
| | | GTGACCCACCAGGGCCTGTCTTCTCCTGTGACCAAGTCCTTCAACA |
| | | GAGGCGAGTGCTGA |
| 46 | TH-115 | ATGGGCTGGTCCTGCATCATCCTGTTCCTGGTGGCTACCGCCACCG |
| | heavy chain | GCGTGCACTCCCAAGTGCAACTACTAGAAAGTGGTGGTGGTCTCGT |
| | IgG1-LALA | CAAACCCGGTGGCTCCCTGCGGCTGAGCTGTGCTACCTCTGGCTAC |
| | nucleic acid | GTGTTCTCCAGATCCTGGATGAACTGGATCCGCCAGGCTCCTGGCC |
| | | AGGGCCTGGAATGGATCGGCTGGATCTACCCTGGAGATGGCGATA |
| | | CCAACTACAACGGCAAGGTGAAGGACCGGTTTACACTGTCTGCCG |
| | | ACAAGGCCAAGAACTCCGTGTACCTGCAGATGAACTCCCTGAGAG |
| | | CCGAGGACACCGCTGTGTACTTCTGCGCCAGAGGCGGATATGACG |
| | | GCTCTCCTTGGCTGTCCTACTGGGGCCAAGGCACCCTGGTGACCGT |
| | | GTCCAGCGCAAGTACTAAGGGTCCAAGTGTGTTTCCACTGGCTCCC |
| | | TCTAGCAAGTCCACCTCTGGCGGCACCGCCGCCCTGGGCTGCCTGG |
| | | TCAAGGACTACTTCCCCGAGCCCGTCACCGTGTCCTGGAACTCCGG |
| | | CGCTCTGACCTCCGGCGTGCATACCTTCCCTGCTGTGCTGCAGTCTT |
| | | CTGGCCTGTACTCCCTGTCCTCCGTCGTGACAGTGCCTTCTAGCTCT |
| | | CTCGGCACACAGACATACATCTGCAATGTGAACCACAAGCCTTCCA |
| | | ACACCAAAGTGGATAAGAAGGTGGAACCTAAGTCCTGCGACAAAA |
| | | CCCACACCTGTCCTCCTTGCCCTGCTCCTGAAGCCGCTGGCGGACC |
| | | TTCTGTGTTTCTGTTTCCCCCAAAGCCCAAAGACACCCTGATGATCT |
| | | CTCGGACACCTGAGGTGACCTGCGTGGTGGTGGATGTGTCTCACGA |
| | | GGACCCCGAAGTGAAGTTCAACTGGTACGTGGACGGCGTGGAAGT |
| | | GCACAACGCCAAGACCAAGCCACGCGAAGAGCAGTACAACTCTAC |
| | | CTACAGAGTGGTGTCCGTGCTCACAGTGCTGCACCAGGACTGGCTG |
| | | AACGGAAAAGAGTACAAGTGCAAGGTCTCTAACAAGGCTCTGCCA |
| | | GCCCCTATCGAGAAGACCATCTCCAAGGCCAAGGGCCAACCTAGA |
| | | GAGCCTCAAGTGTACACCCTGCCTCCTAGCCGGGACGAGCTGACCA |
| | | AGAACCAGGTTTCCCTGACCTGTCTGGTGAAAGGCTTCTACCCCTC |
| | | CGATATCGCCGTGGAATGGGAGTCCAACGGCCAGCCTGAGAACAA |
| | | CTACAAGACCACCCCTCCTGTGCTGGACTCCGATGGATCCTTCTTC |
| | | CTGTATAGCAAGCTGACCGTGGACAAGTCCAGATGGCAGCAGGGC |
| | | AACGTGTTCAGCTGTTCCGTGATGCACGAGGCCCTGCATAATCACT |
| | | ACACCCAGAAGTCTCTGAGCCTGTCTCCTGGCAAGTGA |
| 47 | TH-115 light | ATGGGCTGGTCCTGCATCATCCTGTTCCTGGTGGCTACCGCCACCG |
| | chain IgG1- | GCGTGCACTCCGAAATCGTGCTAACTCAAAGTCCAGATTTTCAATC |
| | LALA | CGTGACCCCTAAAGAGAAGGTCACAATCACCTGTAAGGCCTCTCA |
| | nucleic acid | GGACATCAACTCCTACCTGTCCTGGTTCCAGCAGAAGCCTGACCAG |
| | | TCCCCTAAGACCCTGATCTATAGAGCCAACCGGCTGGTGGACGGCG |
| | | TGCCAAGCAGATTCTCCGGCTCTGGATCTGGCCAGGATTTTACACT |
| | | GACCATCAACAGCCTGGAAGCCGAGGATGCTGCTACCTACTACTGC |
| | | CTGCAGTACGACGAGTTCCCCTTCACCTTCGGCGGCGGCACCAAGG |
| | | TGGAAATCAAGAGAACCGTGGCCGCTCCTTCTGTGTTCATCTTCCC |
| | | TCCTTCCGACGAGCAGCTGAAGTCCGGCACCGCTTCCGTGGTGTGC |
| | | CTGCTGAACAACTTCTACCCTAGAGAGGCCAAGGTGCAGTGGAAG |
| | | GTGGACAACGCCCTGCAGTCTGGCAACTCCCAAGAGTCCGTCACCG |
| | | AGCAGGACTCCAAGGACTCTACCTACTCCCTGTCCTCCACCCTGAC |
| | | ACTGAGCAAGGCCGACTACGAGAAGCACAAGGTGTACGCCTGCGA |
| | | GGTGACCCACCAGGGCCTGTCTTCTCCTGTGACCAAGTCCTTCAAC |
| | | AGAGGCGAGTGCTGA |
| 48 | peptide | TTIYY |
| | motif bound | |
| | by AGR2 | |
| 49 | AGR2 ER | KTEL |
| | retention | |
| | signal | |
| 50 | β-actin | gggTCAgAAggATTCCTATg |
| | forward | |
| | primer | |
| 51 | β-actin | ggTCTCAAACATgATCTggg |
| | reverse | |
| | primer | |
| 52 | Fibronectin | CgAAgCCgggAAgAgCAAg |
| | forward | |
| | primer | |
| 53 | Fibronectin | cgTTCCCACTgCTgATTTATCTg |
| | reverse | |
| | primer | |
| 54 | α-SMA | CCTgACgggCAggTgATC |
| | forward | |
| | primer | |
| 55 | α-SMA | ATgAAAgATggCTggAAgAgAgTCT |
| | reverse | |
| | primer | |
| 56 | Col1A1 | gAgTACTggATCgACCCTAACCAA |
| | forward | |
| | primer | |
| 57 | Col1A1 | ACACAggTCTgACCTgTCTCCAT |
| | reverse | |
| | primer | |
| 58 | TGF β | CCCgAAgCggACTACTATgCT |
| | forward | |
| | primer | |
| 59 | TGF β | gTTTTCTCATAgATggCgTTgTTg |
| | reverse | |
| | primer | |

### EXAMPLES

The present invention is further illustrated by the following examples.

TABLE 1
Sequences of the antibodies of the examples.

| Anti- | | | Heavy | Heavy chain | Light |
|---|---|---|---|---|---|
| body | VH | VL | chain IgG1 | IgG1-LALA | chain |
| name | sequence | sequence | sequence | sequence | sequence |
| TH-101 | SEQ ID | SEQ ID | | SEQ ID | SEQ ID |
| | NO: 16 | NO: 19 | | NO: 30 | NO: 33 |
| TH-104 | SEQ ID | SEQ ID | | SEQ ID | SEQ ID |
| | NO: 17 | NO: 19 | | NO: 31 | NO: 33 |
| TH-106 | SEQ ID | SEQ ID | SEQ ID | SEQ ID | SEQ ID |
| | NO: 16 | NO: 20 | NO: 27 | NO: 30 | NO: 34 |
| TH-109 | SEQ ID | SEQ ID | | SEQ ID | SEQ ID |
| | NO: 17 | NO: 20 | | NO: 31 | NO: 34 |
| TH-115 | SEQ ID | SEQ ID | | SEQ ID | SEQ ID |
| | NO: 18 | NO: 21 | | NO: 32 | NO: 35 |

### Example 1: Anti-AGR2 Humanized Monoclonal Antibody Production

### Material and Methods

### Plasmid Preparation

Target DNA sequence was designed, optimized and synthesized. The complete sequence was sub-cloned into GenScript's proprietary vector. Transfection grade plasmid was maxi-prepared for TurboCHO-HT cell expression.

### Cell Culture and Transient Transfection

The cells were maintained at 37° C. with 5% CO₂ on an orbital shaker. One day before transfection, the cells were seeded at an appropriate density. On the day of transfection, DNA and Reagent were mixed at an optimal ratio and then added into cells ready for transfection. Approximately 24 hours post-transfection, feed was added to each sample.

### Purification and Analysis

Cell culture broth was centrifuged and followed by filtration. Filtered cell culture supernatant was loaded onto an affinity purification column at an appropriate flowrate. After washing and elution with appropriate buffers, the eluted fractions were pooled and buffer exchanged to the final formulation buffer. The purified protein was analyzed by SDS-PAGE and SEC-HPLC analysis to determine the molecular weight and purity. The concentration was determined by A280 method (i.e., absorbance of a protein solution at 280 nm).

### Results

TH-101 IgG1-LALA, TH-104 IgG1-LALA, TH-106 IgG1-LALA, TH-106 IgG1 and TH-109 IgG1-LALA antibodies were easily produced. Additionally, all of TH-101 IgG1-LALA, TH-104 IgG1-LALA, TH-106 IgG1-LALA, TH-106 IgG1 and TH-109 IgG1-LALA antibodies had a better yield than Agtuzumab IgG1-LALA (as shown on Table 2 below). However, TH-115 IgG1-LALA antibody had a lower yield compared to the other humanized antibodies (as shown on Table 2).

TABLE 2
Production characteristics of Agtuzumab IgG1-LALA, TH-101
IgG1-LALA, TH-104 IgG1-LALA, TH-106 IgG1-LALA, TH-106 IgG1,
TH-109 IgG1-LALA, and TH-115 IgG1-LALA antibodies.

| | | Purity | Purity | | |
|---|---|---|---|---|---|
| | Concen- | of SDS- | by SEC- | | |
| | tration | PAGE | HPLC | Total | |
| Name | (mg/ml) | (%) | (%) | (mg) | Yield |
| Agtuzumab IgG1-LALA | 0.69 | ≥95 | 94 | 1.1 | 0.05 |
| TH-101 IgG1-LALA | 3.24 | ≥95 | 98 | 5.18 | 0.25 |
| TH-104 IgG1-LALA | 3.19 | ≥95 | 97 | 5.1 | 0.25 |
| TH-106 IgG1-LALA | 2.77 | ≥95 | 98 | 4.43 | 0.22 |
| TH-106 IgG1 | 3.22 | ≥95 | 99 | 5.15 | 0.25 |
| TH-109 IgG1-LALA | 2.96 | ≥95 | 98 | 4.73 | 0.23 |
| TH-115 IgG1-LALA | 1.40 | ≥95 | 96 | 2.24 | 0.11 |

### Example 2: Epitope Mapping

### Material and Methods

### Generation of DMS DNA Libraries

DMS libraries of human AGR2 protein were generated by PCR, each mutated position carries a degenerated “NNS” or “NNK” codon encoding 20 amino acids/32 codons.

### Construction of the Expression Plasmid of the Antigen

The gene corresponding to human AGR2 WT (SEQ ID NO: 1) was synthesized and cloned into a plasmid allowing expression on the surface of galactose-inducible yeasts. In this construct, the expressed antigen (i.e., human AGR2 WT) carries a C-terminal HA tag. Expression plasmids are then transformed into the yeast strain S. cerevisiae EBY100.

### Induction of Antigen Expression in Yeast Surface Display

Induction of the transformed yeasts in SG-CAA induction medium (6.7 g/L yeast nitrogen base without casamino acids, 20 g/L, 5 g/L casamino acids, 100 mM sodium phosphate, pH 6.0) allows the expression of the antigen (i.e., human AGR2 WT) on the yeast surface.

### Flow Cytometry Sorting

10⁷ induced cells were washed with 1 mL of PBSF (PBS with 0.1% BSA). The cells were then resuspended in an appropriate volume of solution containing 3 nM of Agtuzumab or 500 pM of TH-106 humanized monoclonal antibody. After 2 hours of incubation at 20° C. with agitation, the cells were washed with 1 mL of ice-cold PBSF (to avoid dissociation). Then, the cells were incubated with anti-human PE fluorescent reporter on ice for 15 minutes. The cells were then sorted on a BD FACSAria™ MIII cytometer using the BD FACSDiva™ software.

### Next Generation Sequencing and Data Analysis

Plasmids from each sorted yeast population were extracted and prepared for sequencing. A two-step PCR was performed: a first step to amplify the region of interest and a second step to add the Illumina adapters needed for sequencing. Sequencing was performed on an Illumina iSeq100 instrument (2×150 bp, 300 cycles) with at least 150,000 reads per population. The data were then processed through an analysis pipeline using dedicated proprietary scripts. Poor quality sequences (Q<30) were removed, then mono-mutants were detected and counted.

### Results

Deep Mutational Scanning (DMS) is a mutagenesis method that aims to perform all possible mono-substitutions on all selected residues within a given protein sequence. The DMS library is obtained in the form of DNA coding for the protein under study (i.e., human AGR2 protein in this case). In this library, each DNA strand contains a codon that is mutated with respect to the parental sequence.

This DMS DNA library is integrated into an expression plasmid specifically designed to express recombinant proteins on the yeast surface. Yeasts are then transformed and induced to allow the expression of the mono-mutated proteins on their surface. This new library (called display library) is screened by flow cytometry using fluorescent reporters to reveal the expression of the protein (anti-tag fluorescent antibody) as well as the binding of the protein to its partner (fluorescent partner).

For epitope mapping, the ideal case is to have two antibodies with compatible epitopes that can bind together on the same antigen. In this way, each of the two antibodies acts as a conformational control of the mutated antigen for the other antibody. Indeed, mono-substitutions made on the antigen can have 4 types of effects:
1. Loss of affinity for the first antibody, while retaining binding for the second: this is a mutation made within the epitope of the first antibody.
2. Loss of affinity for the second antibody, while retaining binding for the first: this is a mutation in the epitope of the second antibody.
3. Loss of affinity for both antibodies: this is a so-called “destructuring” mutation which affects the conformation of the antigen and thus prevents the binding of both antibodies.
4. No effect: the mutation is not present in the epitope of one of the two antibodies and does not cause a significant change in the conformation of the antigen.

Following flow cytometry analysis, the yeast population that has lost affinity for the antibody of interest while retaining binding for the second antibody is sorted. The plasmids contained in this yeast population are extracted and sequenced by high-throughput sequencing. Analysis of the sequencing data allows the identification of mutations that have affected the binding of the antibody to its target. Thus, this analysis allows to identify the important positions on the antigen for the binding of the antibody of interest: i.e., its epitope.

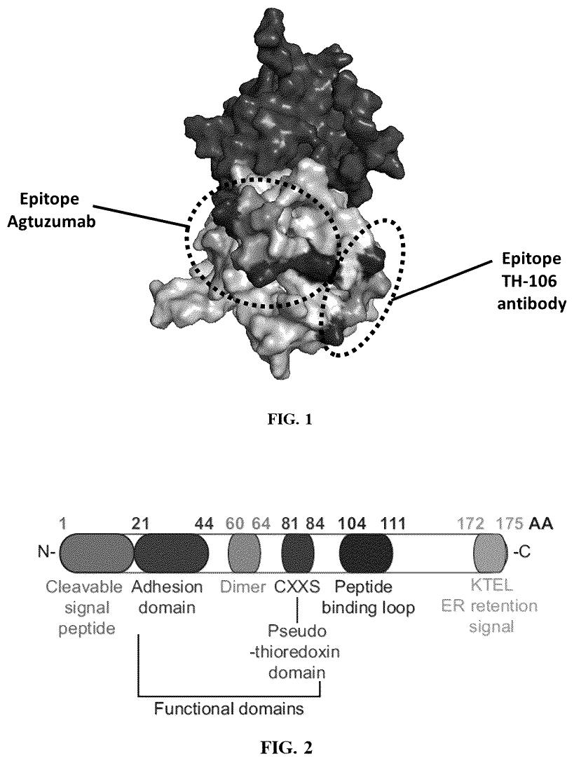

The epitope bound by Agtuzumab and by TH-106 humanized monoclonal antibody were identified using DMS. As shown on FIG. 1, the epitope bound by Agtuzumab and the epitope bound by TH-106 humanized monoclonal antibody are located in two distinct domains of the human AGR2 protein. Agtuzumab's epitope contains as most important amino acid residues: the histidine residue at position 117 (H117), the aspartic acid residue at position 121 (D121) and the tyrosine residue at position (124). On the other hand, the epitope bound by TH-106 humanized monoclonal antibody contains as most important amino acid residues: the proline residue at position 82 (P82) and the glutamic acid residue at position 153 (E153). As shown on FIG. 2, showing the structure of the AGR2 protein, the proline residue at position 82 is located in the pseudo-thioredoxin domain. Therefore, the epitope bound by TH-106 humanized monoclonal antibody is located in the catalytic domain of the human AGR2 protein, while amino acid residues 117, 121 and 124 bound by Agtuzumab are not located in a functional domain (as shown on FIG. 2). This suggests that the TH-106 humanized monoclonal antibody would be able to inhibit AGR2 catalytic activity, while Agtuzumab would not.

### Example 3: Cross-Reactivity and Specificity of the Anti-AGR2 Humanized Monoclonal Antibodies

### Material and Methods

### ELISA

ELISA optimized plates were coated with human AGR2 WT (SEQ ID NO: 1), mouse AGR2 (SEQ ID NO: 2), dog AGR2 (SEQ ID NO: 6), macaque AGR2 (SEQ ID NO: 3), human AGR2 E60A (SEQ ID NO: 7), human AGR3 or BSA at a concentration of 1 μg/mL in 50 μL per well and incubated overnight at 4° C. Then, plates were washed using 300 μL per well of PBS with 0.05% of Tween 20. Plates were blocked using 150 μL per well of PBS with 2.5% milk during 1 hour at room temperature. Then, plates were washed using 300 μL per well of PBS with 0.05% of Tween 20. Agtuzumab IgG1-LALA, TH-101 IgG1-LALA, TH-104 IgG1-LALA, TH-106 IgG1-LALA, TH-106 IgG1, TH-109 IgG1-LALA, and TH-115 IgG1-LALA antibodies were diluted at an initial concentration of 10 μg/mL and serially diluted at ¼ down to 0.15 ng/mL. Antibodies were added to the plates and incubated for 2 hours at room temperature. Plates were washed three times using 300 μL per well of PBS with 0.05% of Tween 20. Anti-human IgG1 conjugated to HRP (Sigma A0170-1 mL, lot: 0000088179) secondary antibody diluted 1/2000 was added to the plates and incubated for 1 hour at room temperature. Detection was performed using TMB (KPL 52-00-01; lot 10602343). Reaction was stopped by H2SO4 solution and optical density (OD) was read at 450 nm.

### Results

The binding capacity toward human AGR2 WT, mouse AGR2, dog AGR2, macaque AGR2, human AGR2 E60A, human AGR3 or BSA was evaluated for each of Agtuzumab IgG1-LALA, TH-101 IgG1-LALA, TH-104 IgG1-LALA, TH-106 IgG1-LALA, TH-106 IgG1, TH-109 IgG1-LALA, and TH-115 IgG1-LALA humanized monoclonal antibodies using ELISA.

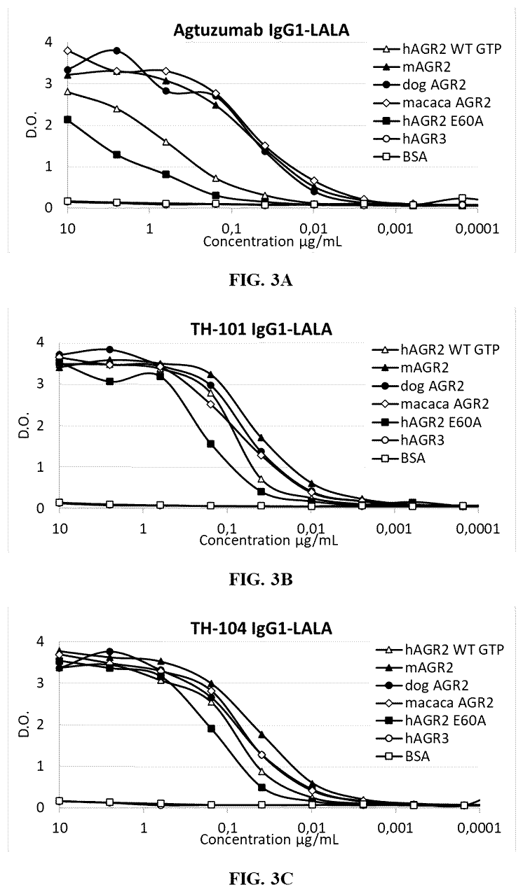

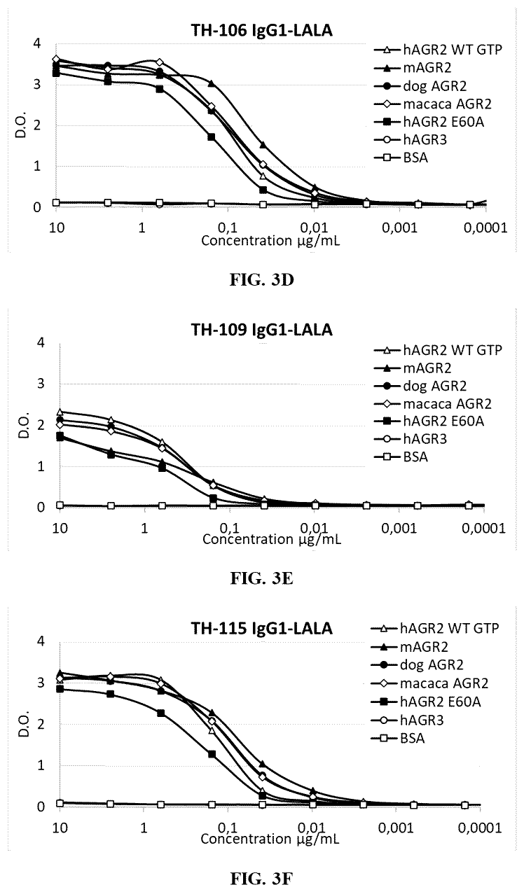

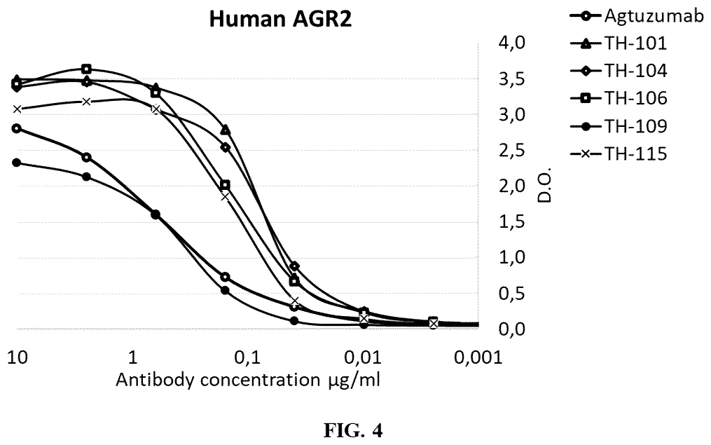

As shown on FIGS. 3A-F, all antibodies were able to recognize and bind both human AGR2 WT and human AGR2 E60A. Additionally, all antibodies were able to cross-react with mouse AGR2, dog AGR2 and macaque AGR2, while none of them was able to bind human AGR3 or BSA used as a control. However, as shown by the EC50 presented in Table 3 below, TH-101, TH-104, TH-106, and TH-115 humanized monoclonal antibodies were better binders than Agtuzumab, in particular for binding human AGR2 WT (FIG. 4) and human AGR2 E60A. On the opposite, TH-109 humanized monoclonal antibody displayed a lower binding capacity than the other humanized monoclonal antibodies.

As shown on Table 4 below, Agtuzumab binds to human AGR2 with an affinity in the nanomolar range. All other antibodies (i.e., TH-101, TH-104, TH-106, TH-109, and TH-115) show a very strong binding to AGR2 with a K_{D} value under 150 pM.

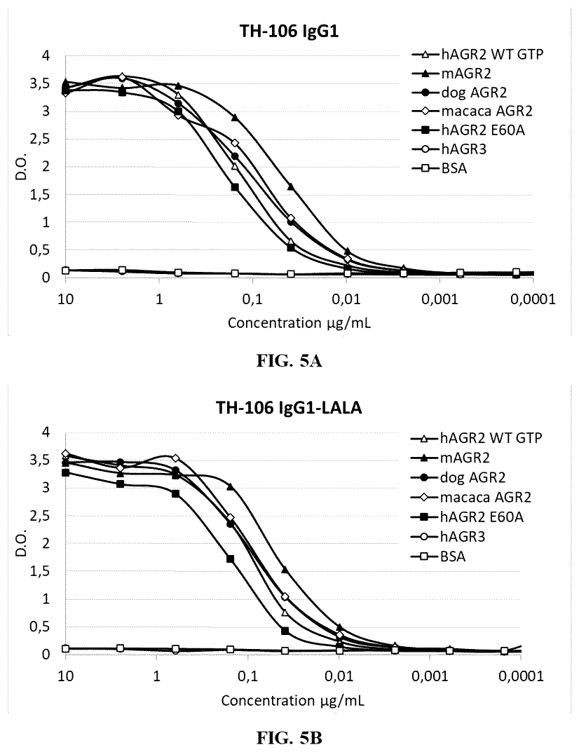

In addition, binding capacity was compared between TH-106 IgG1-LALA and TH-106 IgG1 antibodies. As shown on FIGS. 5A-B and Table 3, scaffold change had a minimal impact on TH-106 humanized monoclonal antibody binding properties.

TABLE 3
EC50 of Agtuzumab IgG1-LALA, TH-101 IgG1-LALA, TH-104 IgG1-LALA,
TH-106 IgG1-LALA, TH-106 IgG1, TH-109 IgG1-LALA, and TH-115 IgG1-
LALA humanized monoclonal antibodies toward human AGR2 WT, mouse
AGR2, dog AGR2, macaque AGR2, human AGR2 E60A, and human AGR3.

| | | | | | Human | |
|---|---|---|---|---|---|---|
| | Human | Mouse | Dog | Macaque | AGR2 | Human |
| EC50 (pM) | AGR2 -WT | AGR2 | AGR2 | AGR2 | E60A | AGR3 |
| Agtuzumab IgG1- | 3073.33 | 353.33 | 453.33 | 400 | 8640 | ND |
| LALA | | | | | | |
| TH-101 IgG1-LALA | 520 | 280 | 420 | 480 | 1240 | ND |
| TH-104 IgG1-LALA | 526.7 | 300 | 480 | 440 | 920 | ND |
| TH-106 IgG1-LALA | 633.3 | 313.3 | 546.7 | 546.7 | 960 | ND |
| TH-106 IgG1 | 853.3 | 300 | 660 | 553.3 | 1100 | ND |
| TH-109 IgG1-LALA | 2373.3 | 2026.7 | 2313.3 | 2153.3 | 3573.3 | ND |
| TH-115 IgG1-LALA | 813.3 | 500 | 600 | 620 | 1286.7 | ND |

TABLE 4
Dissociation constant (K_{D}), association rate constant
(ka) and dissociation rate constant (Kdis) towards
human AGR2 measured for Agtuzumab IgG1-LALA, TH-101
IgG1-LALA, TH-104 IgG1-LALA, TH-106 IgG1-LALA, TH-
109 IgG1-LALA, and TH-115 IgG1-LALA antibodies.

| | Soluble | K_{D} | ka | Kdis |
|---|---|---|---|---|
| Immobilized ligand | analyte | (M) | (1/Ms) | (1/s) |
| Agtuzumab IgG1-LALA | hAGR2 | 2.47E−09 | 3.42E+05 | 8.46E−04 |
| TH-101 IgG1-LALA | hAGR2 | 1.16E−10 | 3.03E+05 | 3.52E−05 |
| TH-104 IgG1-LALA | hAGR2 | 2.78E−11 | 2.96E+05 | 8.23E−06 |
| TH-106 IgG1-LALA | hAGR2 | 3.27E−12 | 3.26E+05 | 1.07E−06 |
| TH-109 IgG1-LALA | hAGR2 | 2.65E−11 | 3.29E+05 | 8.74E−06 |
| TH-115 IgG1-LALA | hAGR2 | 6.30E−11 | 3.08E+05 | 1.94E−05 |

### Example 4: Thermal Stability of the Anti-AGR2 Humanized Monoclonal Antibodies

### Material and Methods

Thermal stability of Agtuzumab IgG1-LALA, TH-101 IgG1-LALA, TH-104 IgG1-LALA, TH-106 IgG1-LALA, TH-106 IgG1, TH-109 IgG1-LALA, and TH-115 IgG1-LALA humanized monoclonal antibodies was evaluated by Differential Scanning calorimetry (DSC).

Data were fitted using the MicroCal PEAQ DSC Analysis software to a non-two-state unfolding model and included buffer subtraction. Subsequently, the thermal transition midpoint (Tm), representing the point at which there is an equal amount of folded/unfolded protein undergoing a transition, was determined. The calorimetric enthalpy (ΔHcal) was calculated from the integrated area under the peak of the transition and represented the total heat energy uptake by the sample undergoing the transition. This heat uptake depended on the concentration of sample in the sample cell of the instrument that was undergoing transition and was a model-free absolute measure of the enthalpy of the process involved.

### Results

As shown on Table 5, all humanized monoclonal antibodies except TH-115 IgG1-LALA antibody displayed a better thermal stability than Agtuzumab.

TABLE 5
Melting temperature (Tm) of the Fab fragments of
Agtuzumab IgG1-LALA, TH-101 IgG1-LALA, TH-104 IgG1-
LALA, TH-106 IgG1-LALA, TH-109 IgG1-LALA, and TH-
115 IgG1-LALA humanized monoclonal antibodies.

| Antibody | Isotype | Tonset | Tm (° C.) |
|---|---|---|---|
| TH-101 | Human IgG1-LALA | 66.19 | 83.28 |
| TH-104 | Human IgG1-LALA | 66.45 | 81.975 |
| TH-106 | Human IgG1-LALA | 65.52 | 82.19 |
| TH-109 | Human IgG1-LALA | 65.51 | 80.36 |
| TH-115 | Human IgG1-LALA | 60.68 | 69.52 |
| Agtuzumab | Human IgG1-LALA | 66.92 | 76.28 |

### Example 5: Effect of the Anti-AGR2 Humanized Monoclonal Antibodies on Monocyte Migration

### Material and Methods

### Monocytes Chemoattraction Assay

Peripheral blood mononuclear cells (PBMCs) were isolated from healthy donors. PBMCs were washed in RPMI 1% FCS (Life Technologies) and placed in the upper part of Boyden chamber systems (5×10⁵ cells/chamber in RPMI 1% FCS; Millipore, France). Boyden chamber upper parts were placed in RPMI containing recombinant human AGR2 or tumor cell culture supernatants containing eAGR2, in the presence or absence of either Agtuzumab or TH-106 humanized monoclonal antibody. Boyden chambers were then incubated at 37° C. for 24 hours. The migrated PBMCs (in the lower part of the Boyden chambers) were collected, washed in PBS, and cells were stained for monocytes, T cell, B cell, and NK cell markers (anti-CD14, -CD3, -CD19, and -CD56, respectively) and analyzed by flow cytometry. Data were then analyzed using FACSDiva (BD Biosciences). The relative number of migrated cells was estimated by flow cytometry by counting the absolute number of cells.

### Results

Monocyte migration induced either by recombinant human AGR2, or tumor cell (Mz-Cha-1) supernatant containing eAGR2 is blocked by both Agtuzumab and TH-106 humanized monoclonal antibody. The residual migration in the presence of TH-106 humanized monoclonal antibody is comparable to the negative control (medium only). However, the blockade is more efficient with the TH-106 humanized monoclonal antibody, as compared to Agtuzumab. These data demonstrate that TH-106 anti-AGR2 humanized monoclonal antibody is capable of inhibiting monocyte migration induced by Mz-Cha-1 conditioned medium.

### Example 6: Effect of the Anti-AGR2 Humanized Monoclonal Antibodies on Myofibroblast Differentiation

### Material and Methods

### Fibroblast to Myofibroblast Differentiation of the CCD-18Co

CCD-18Co cells were seeded in six-well plates (1×10⁶ cells/well) and grown for 48 hours before treatments. Endoplasmic reticulum (ER) stress was induced on CCD-18Co cells with 10 μg/mL Tunicamycin (Tm, Sigma, solubilized in DMSO) and DMSO was used as control condition (at the same final concentration) to monitor the response of CCD-18 to Tm. The CCD-18Co cell differentiation-positive control was obtained using a 48 hours treatment with 10 ng/ml of TGF-β1 (R&D systems) added in the media of CCD-18Co cells (after 24 hours of serum starvation and addition of 1% FBS at the time of stimulation). The supernatants of HT-29 cells, pre-conditioned or not by Tm (thus, subject or not to a transient ER stress) and collected 8, 24, and 32 hours after the media change, were applied on CCD-18Co cells, as inducing conditions, for further 48 hours of incubation. The HT-29 supernatant was used without any freezing cycle. The capacity of recombinant human AGR2 (rAGR2) to induce fibroblast to myofibroblast differentiation was investigated by application of 40 ng/mL of rhAGR2 in the media of CCD-18Co cells (after 24 hours of serum starvation and addition of 1% FBS at the time of stimulation), the control being the same condition without rhAGR2 supplementation. The impact of AGR2 blockade, using Agtuzumab or TH-106 anti-AGR2 humanized monoclonal antibody, supplemented to the conditions with rhAGR2 or to the HT-29 supernatant pre-conditioned by Tm, was evaluated. The HT-29 and CCD-18Co cells were harvested and treated for either total protein extracts [stored at −20° C.] or for RNA extractions [stored at −80° C.]. HT-29 supernatants collected for analysis were stored at −20° C. Samples were used for immunofluorescence analyses, western blot, and RNA extraction and RT-qPCR.

### Results

Fibroblasts can differentiate into myofibroblasts when exposed to AGR2, which is at the origin of fibrosis. Myofibroblast differentiation is induced by eAGR2. Both Agtuzumab and TH-106 humanized monoclonal antibody are capable of blocking this differentiation. However, blockade of myofibroblast differentiation is more efficient with TH-106 humanized monoclonal antibody, as compared to Agtuzumab.

### Example 7: In Vivo Efficacy of the Anti-AGR2 Monoclonal Antibodies in Acute DSS Model

### Material and Methods

### Animal Model and Colitis Induction

Animal experiments were performed in accredited facilities at Institut Pasteur in Lille (licence no B59-35009) according to governmental guidelines (articles R214-87 à R214-137 code rural update 13 Feb. 2013 according to European directive 2010/63/UE) and those of the Nord-Pas de Calais Ethical Committee for animal use. Animals were housed five per cage and had free access to standard mice chow and tap water.

For this study, C57Bl/6 mice were used. For colitis induction, C57Bl/6 mice received 2.5% of dextran sulfate sodium (DSS) (45 kD; MP Biomedicals, Ref 160110) in their drinking water for 5 days (from day 0 to day 5) followed by a regime of 3 or 7 days of regular water. At day 8 after beginning of DSS administration, a peak of inflammation was reached. Mice were euthanized at day 12.

110 C57Bl/6 mice were allocated to the following groups, as described in Table 6 below.

TABLE 6
Groups of mice for in vivo study of DSS-induced acute colitis.

| | | | | Number |
|---|---|---|---|---|
| Groups | Route | Dosing | Volume | of mice |
| Non-DSS control + | IP | D 0, D 2, D 4, D 6, | 100 μL | 5 |
| vehicle | | D 8 & D 10 | | |
| DSS + vehicle | IP | D 0, D 2, D 4, D 6, | 100 μL | 15 |
| | | D 8 & D 10 | | |
| DSS + anti-AGR2 | IP | D 0, D 2, D 4, D 6, | 100 μL | 15 |
| 5 μg/mouse | | D 8 & D 10 | | |
| DSS + anti-AGR2 | IP | D 0, D 2, D 4, D 6, | 100 μL | 15 |
| 10 μg/mouse | | D 8 & D 10 | | |
| DSS + anti-AGR2 | IP | D 0, D 2, D 4, D 6, | 100 μL | 15 |
| 20 μg/mouse | | D 8 & D 10 | | |
| DSS + anti-AGR2 | IP | D 0, D 2, D 4, D 6, | 100 μL | 15 |
| 40 μg/mouse | | D 8 & D 10 | | |
| DSS + anti-AGR2 | IV | D 0, D 2, D 4, D 6, | 100 μL | 15 |
| 5 μg/mouse | | D 8 & D 10 | | |
| DSS + anti-AGR2 | IV | D 0, D 2, D 4, D 6, | 100 μL | 15 |
| 20 μg/mouse | | D 8 & D 10 | | |

### Antibody Treatment

TABLE 7
Sequences of the anti-AGR2 antibody used in Example 7.

| | |
|---|---|
| V_{H}-CDR1 | SEQ ID NO: 8 |
| V_{H}-CDR2 | SEQ ID NO: 10 |
| V_{H}-CDR3 | SEQ ID NO: 12 |
| V_{L}-CDR1 | SEQ ID NO: 13 |
| V_{L}-CDR2 | SEQ ID NO: 14 |
| V_{L}-CDR3 | SEQ ID NO: 15 |

The anti-AGR2 monoclonal antibody was administered at a dose ranging from 5 μg to 40 μg per mouse by intraperitoneal (IP) or intravenous (IV) route, every two days, starting the day of the first DSS administration in the drinking water until the day of euthanasia, occurring 7 days after the last DSS administration (corresponding to 6 administrations in total (D0, D2, D4, D6, D8 & D10), which corresponds to a total of 6 injections.

### Clinical Examination

### Mortality and Clinical Signs

Each animal was checked for mortality once a day for the duration of this experiment. The body weight of each animal was recorded every day from day 0 (first day of colitis induction by DSS) to day 12 (euthanasia time point). The body weight was homogenous between the different groups before colitis induction.

### Disease Activity Index (DAI)

Different parameters were monitored daily for the duration of the experiment to evaluate the effects of the anti-AGR2 antibody on the colonic inflammation. The Disease Activity Index (DAI) is a simple scoring system used to determine the severity of colitis in mice. The Disease Activity Index (DAI) was determined at euthanasia (D12) based on the evolution of body weight, the stool consistency and the presence of blood in the feces evaluated using the Hemoccult test. Briefly, DAI was assessed by an investigator blinded to the protocol according to a standard scoring system. Body weight (BW), stool consistency (with a score from 0-3, 0=normal, 1=soft, 2=Diarrhea, 3=watery diarrhea, and visible presence of blood (rectum of mice) were recorded daily. At euthanasia, the presence of Occult Blood (OB) was recorded using the hemoccult method. Loss in BW was scored as: 0, no weight loss; 1, weight loss of <10% from baseline; 2, weight loss of >10% from baseline. For stool consistency, a score of 0 was assigned for well-formed pellets, 1 for pasty and semi formed stools that did not adhere to the anus, and 2 for liquid stools that adhered to the anus. For OB, a score of 0 was assigned for no blood, 1 for positive OB or for gross bleeding. These scores were added together. BW loss was calculated as the percentage difference between the original BW (day 0) and the BW on any particular day.

### Ratio Weight/Size of the Colon

After euthanasia, the colon was carefully dissected and its weight and size were measured. Indeed, a decrease of the size of the colon was induced by severe inflammation and an increase of colon weight was also observed due to edema/inflammatory infiltrate caused by a severe inflammation.

### Blood and Tissue Sampling

Serum from each animal was collected at the time of euthanasia by cardiac puncture, placed in a 1.1 ml Z-Gel micro tube (Sarstedt). Tubes were inverted 5 times and samples were then allowed to clot for 1 hour at ambient temperature. Tubes were centrifuged at 2300×G for 10 minutes at 20° C.

Serum (supernatant) were collected into a fresh pre-chilled tube and stored at −80° C. until analysis. Samples from distal colon were divided in 4 parts and snap-frozen (one part for protein analysis, one part for histological assessment, one part for MPO measurement, and the other one for mRNA extraction in 500 μl of RNAlater). Spleen, a part of liver (Right lobe), MLNs (when possible), and a part of distal ileum were snap-frozen and one part of the distal ileum taken off for histological assessments.

### Assessment of Histological Colonic Lesion

To assess the level of inflammation, colon samples embedded in paraffin were analyzed. For the histological evaluation, sections of colonic tissues (4 μm) and distal ileum were stained with May-Grunwald-Giemsa and evaluated. A multiparametric scoring (0 to 18), as described in Dieleman et al., (1998), was performed blindly by two investigators. The histological examination graded the severity and extent of inflammation, the intensity of cellular infiltrate in the mucosa, its extension in sub-mucosa layers, and the presence of epithelial lesions.

### MPO Quantification

Myeloperoxidase (MPO) is an enzyme contained in polymorphonuclear neutrophil primary granules and used as a marker of neutrophil infiltration. MPO protein level is a quantitative and objective marker used to evaluate neutrophil recruitment in tissues and, indirectly, colonic inflammation mediated by neutrophils. The levels of MPO were quantified by ELISA (ref HK210-01, Clinisciences), per the manufacturer's recommendations, in colon samples. MPO activity was measured to monitor the degree of neutrophil infiltration in the colonic lesions during chemically induced colitis. Colon specimens were homogenized with an Ultra Turrax T8 (Ika-Werke, Staufen, Germany) in a phosphate buffer (pH 6.0) containing 0.5% hexadecyltrimethyl ammonium and subjected to two sonication and freeze-thaw cycles. The suspensions were centrifuged at 14,000×g for 15 min at 4° C. and the supernatants were reacted with 1 mg/mL o-dianisidine hydrochloride and 0.0005% hydrogen peroxide. The optical density of each sample was read at 450 nm with a Versamax microplate reader (MDS Analytical Technologies, Saint-Grégoire, France). One unit of MPO activity was defined as the amount that degraded 1 μmol peroxidase per minute at 25° C. The results were expressed as absorbance per total quantity of proteins determined by the Bradford method (Pineton de Chambrun G et al. Mucosal Immunol. 2014).

### Cytokines and Chemokines Analysis

Analytes in mice serum were quantified by ELISA method using Cytokine & Chemokine Convenience 26-Plex Mouse ProcartaPlex™ Panel 1 (ThermoFischer), TGF beta 1 Mouse ProcartaPlex™ Simplex Kit (ThermoFischer) and ProcartaPlex Mouse Basic Kit (2nd part of TGF-b1 kit) (ThermoFischer). The following analytes were quantified: GM-CSF, IFN-γ, IL-1 B, IL-2, IL-4, IL-5, IL-6, IL-9, IL-10, IL-12p70, IL-13, IL-17A (CTLA-8), IL-18, IL-22, IL-23, IL-27, TNF α, Eotaxin (CCL11), GRO α (CXCL1), IP-10 (CXCL10), MCP-1 (CCL2), MCP-3 (CCL7), MIP-1 α (CCL3), MIP-1 β (CCL4), MIP-2 α (CXCL2), RANTES (CCL5) and TGF-β1. The Bio-Plex®200 system, a flow-based dual-laser system for simultaneously identifying and quantitating up to 100 different analytes in a single biomolecular assay (xMAP technology) was used. Analyte concentration is proportional to the mean fluorescence intensity (MFI) collected from 50 beads per region, one specific microparticle region being assigned per analyte, and back-calculated from interpolation with the calibration curve.

Analytes' mRNA levels were assessed by qRT-PCR. Mouse colon tissue samples were obtained from mice and stabilized in RNALater (ThermoFisher Scientific, AM7020) and stored at −80° C. Tissue was thawed, cleaned from excess RNALater in PBS and underwent bead tissue homogenization using a MiniLys tissue homogenizer (Bertin Technologies, Ref. P000673-MLYSO-A) with 1.4 mm ceramic beads in 2 mL tubes (Bertin Technologies, CK14-2 mL, Ref. P000933-LYSKO-A) filled with 350 μL Tripure Isolation Reagent (Sigma-Aldrich, Cat. 11667157001) at 5000 rpm for 3 cycles of 15 seconds. 650 μL of Tripure were added to the homogenized tissue and extraction was performed using the Maxwell® RSC miRNA Plasma and Serum Kit (Promega, Cat. AS1680) in combination with the Maxwell RSC48 Instrument (Promega, Cat. AS8500). Pre-treatment of samples is done following a protocol developed in-house. Briefly, 200 μL of Bromo-Chloro-Propane (BCP) (Sigma-Aldrich, Cat. B9673) are added to 1 mL of sample homogenized in Tripure, and mixed by vortexing for 10-15 seconds until solution bleaches. Afterwards, a 5 minute incubation at room temperature and centrifugation at 12,000 rcf on a benchtop centrifuge for 15 minutes at 4° C. were performed. The aqueous phase containing the RNA (approximately 500 μL) is then transferred to a new 1.5 mL eppendorf tube and 230 uL of Lysis Buffer C from Maxwell® RSC miRNA Plasma and Serum Kit is added to it. Mixed by vortexing for 5 seconds. Cartridges preparation from Maxwell RSC miRNA kit and Maxwell RSC 48 Instrument preparation and run are done following the steps outlined by Promega. Elution was done in 75 uL of nuclease free water. Reverse transcription of 2.5 ug of RNA per sample was performed using the SuperScript™ IV VILO™ Master Mix (ThermoFisher Scientific, Cat. 11756050) following the user guide provided by the manufacturer. Briefly, 4 uL of Master Mix are combined on ice with 2.5 ug of total RNA and nuclease-free water up to 20 uL and mixed by vortexing. The reverse-transcription reaction is then run on a thermocycler under the following program: 25° C./10 mins.; 50° C./10 mins.; 85° C./5 mins. RT-PCR was performed using custom designed Taqman Array Fast 96-well plates using 45 ng of cDNA per well following the User Guide provided by ThermoFisher Scientific. Briefly, the cDNA and nuclease-free water are combined with Taqman Fast Advance Master Mix (Cat. 4444963) in a microcentrifuge tube, which is then vortex mixed. 10 uL of the cDNA-Master Mix is added to the wells of the Taqman Array Fast plate, which is then sealed and centrifuged. RT-PCR run was performed under following cycling conditions in a Quantstudio 5 Real-Time PCR System, 96-well 0.2 mL (Cat. A28139):1 cycle (50° C./2 mins.; 95° C./20 secs.), 40 cycles (95° C./1 sec.; 60° C./20 secs.).

### Immune Infiltrate

The quantity of T-cells, macrophages and neutrophils in mice colon, using immunohistochemistry (IHC) and digital morphometric analysis was performed. Paraffin cross sections, approximately 4 microns thick, were cut, put on glass slides and stained with CD3 for T-cells, F4/80 for macrophages and LY6G for neutrophils, using IHC. The slides were subjected to histological evaluation and digital morphometry. Prepared stained slides were scanned by a KF-BIO-40 scanner. Image Pro Ver 10. was used to count the number of stained cells per colon cross section. Only the darkest brown stain cells were considered positive and counted using the Smart Segmentation method, based on color, background and morphology. The table below shows the number of positive cells per cross section, for each staining. The methodology was the same for all three stains. Statistical analysis was conducted using nonparametric Kruskal-Wallis test followed by Mann-Whitney U test. Significance level was considered at p<0.05.

### Statistical Analysis

All comparisons were analyzed using the Permutation Test for two independent samples, the most powerful statistical test adapted for small number of samples. Statistics were calculated using the StatXact software (Cytel Inc, Cambridge, MA, USA). Differences were considered statistically significant if the p value was <0.05.

### Results

In order to evaluate the anti-inflammatory properties and/or properties on wound healing of the anti-AGR2 monoclonal antibody, 4 doses of anti-AGR2 monoclonal antibody were administered by intraperitoneal route, or 2 doses by intravenous route, in the model of acute colitis induced by dextran sulfate sodium (DSS) in C57Bl/6 mice.

### Mortality

Mortality in each group of mice was monitored every day. A very low mortality rate was recorded in this study. One mouse died in the group of DSS mice receiving the vehicle, one in the group of mice receiving the anti-AGR2 antibody by IP at the dosage of 5 ug and 20 ug, 2 mice died in the group of DSS mice receiving the anti-AGR2 antibody by IP at the dosage of 40 ug and the dosage of 20 ug by IV. 3 mice died in the group of DSS mice receiving the anti-AGR2 antibody at the dosage of 10 μg/mouse by IP. The death can be attributed to the severe colitis induced by DSS, leading to an important body weight loss up to 20% vs their initial body weight. However, this result indicates that the intraperitoneal or intravenous administration of the AGR2 every two days is safe even under inflammatory conditions.

### Disease Activity Index (DAI)

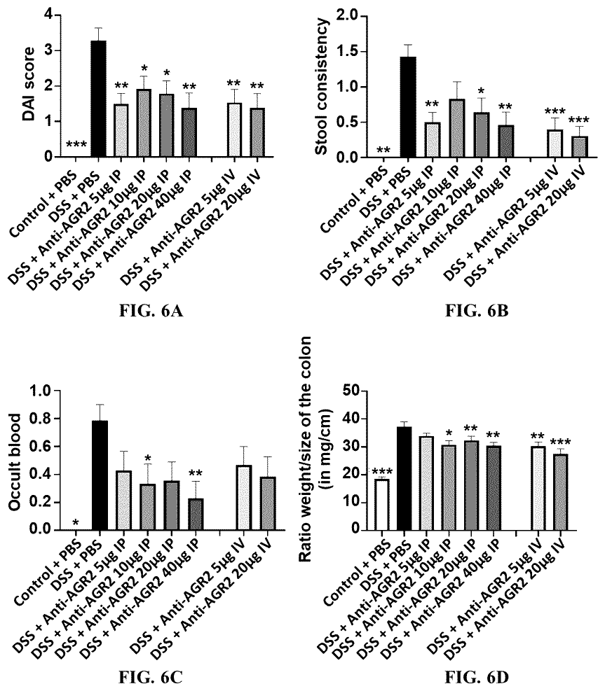

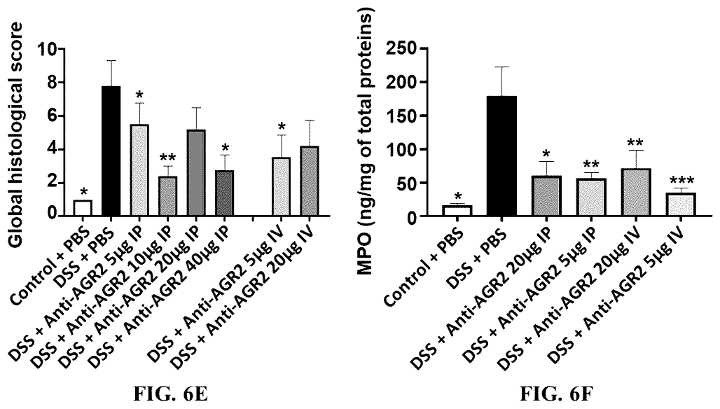

A significant improvement of the DAI score was observed in all the groups of DSS mice receiving the anti-AGR2 antibody at the different tested dosages both by intraperitoneal (IP) and intravenous (IV) administration (FIG. 6A). The anti-AGR2 antibody, both by intraperitoneal or intravenous route, exerted strong improvement of the clinical parameters from 42% to 58%.

The improvement of the DAI score can be explained by analyzing independently the different parameters (i.e., evolution of body weight, stool consistency, and presence of blood in the feces).

### Stool Consistency

In colitic mice receiving the vehicle, a significant increase of the score of stool consistency was recorded, indicating that mice suffered from a strong colitis inducing diarrhea, illustrated by a score of 1.43±0.17 compared to healthy control mice without any sign of colitis and having normal feces consistency (score of 0, p=0.001634) (FIG. 6B).

A significant improvement of the score of stool consistency was recorded in all the groups of DSS mice receiving the anti-AGR2 antibody by IV or IP, except for the group of DSS mice receiving anti-AGR2 at the dosage of 10 μg by IP, in which an important decrease of 42% of the score of stool consistency was still observed (FIG. 6B).

For all the other groups receiving the anti-AGR2 antibody by IP showing a significant improvement of the stool consistency, the percentage of improvement was between 55% to 68%. A higher percentage of improvement was recorded in the groups receiving the anti-AGR2 antibody by IV, with a decrease of stool consistency score of 72% for the dose of 5 μg/mouse IV, and 78% for the dose of 20 μg/mouse by IV (FIG. 6B).

### Presence of Blood in the Feces

In DSS mice receiving the vehicle, a significant increase of the score of presence of occult blood in feces was recorded, indicating that mice have suffered from a strong colitis compared to healthy control mice without any sign of colitis (score of 0.79±0.11 vs 0.00±0.00, p=0.01144, respectively) (FIG. 6C).

A significant decrease of the presence of occult blood in the feces was recorded in the groups of DSS mice receiving the anti-AGR2 antibody by IP at the dosage of 10 μg/mouse (0.33±0.14 vs 0.79±0.11, p=0.04474) and at the dosage of 40 μg/mouse (0.23±0.12 vs 0.79±0.11, p=0.007) compared to the group of DSS mice receiving the vehicle (FIG. 6C). This corresponds to a mean decrease of the presence of occult blood of 58% for the dosage of 10 μg/mouse and up to 70% for the dosage of 40 μg/mouse IP.

A trend to decrease was observed for the DSS mice receiving the IP administration of the anti-AGR2 antibody at the dosages of 5 and 20 μg/mouse. Regarding the IV administration of anti-AGR2 antibody, a trend to decrease the presence of occult blood was observed in DSS mice at the dosage of 5 μg or 20 μg (41% and 52% decrease respectively) (FIG. 6C).

These results indicated that the anti-AGR2 antibody improved the clinical parameters of colitis by decreasing significantly the presence of occult blood with a higher efficacy of the intraperitoneal route of administration and in a dose-dependent manner. Anti-AGR2 antibody also improved the stool consistency but with a higher efficacy for DSS-treated mice receiving the anti-AGR2 antibody by IV route.

### Ratio Weight/Size of the Colon

At euthanasia (day 12), colons were carefully dissected and their size was recorded. The luminal content was then removed from the colon before weighting the colon. Indeed, a decrease in the size of the colon is induced by severe inflammation and an increase of the colon weight is also observed due to edema/inflammatory infiltrate caused by severe inflammation.

In healthy mice, the mean size of the colon was 8.80±0.17 cm and the mean weight was 163.00+4.06 mg. The ratio of the weight/size of the colon was significantly increased in the group of DSS mice receiving the vehicle when compared to the healthy control group without colitis with, respectively, a ratio weight/size of the ratio of 37.30±1.68 vs 18.56±0.70, p=0.00033 (FIG. 6D). This result confirmed that colonic inflammation remained high 7 days after the last DSS administration in the C57BL/6 genetic background.

A significant decrease of the ratio weight/size of the colon was observed in the treated groups of mice receiving the anti-AGR2 antibody by IP at the dosages of 10, 20 and 40 μg/mouse (FIG. 6D). IV administration of the anti-AGR2 antibody at the dosages of 5 and 20 μg/mouse compared to DSS mice receiving the vehicle induced a significant decrease of the colon weight/length ratio (FIG. 6D).

These results indicated a strong anti-inflammatory effect of the anti-AGR2 antibody administered by both IP and IV route.

### Evaluation of Colonic Inflammation at the Histological Level

Evaluation of inflammation and colonic lesions at the histological level was performed according to a validated score for the colitis induced by DSS. Results are expressed as a mean+SEM score. As described above, May-Grunwald-Giemsa-stained sections of colonic tissue were evaluated for inflammation using a multiparametric scoring system (0-18). The system assessed the severity of inflammation (0-3) and extent of inflammation (0-3), the regeneration level (0-4), crypt damage (0-4), and the percentage of involvement (extension) (1-4).

Seven days after the last DSS administration (day 12), a sustained and significant colonic inflammation was still recorded at the histological level in the groups of DSS mice receiving the vehicle compared to the healthy control group without colitis (7.79±1.53 vs 1.00±0.00 p=0.02843) (FIG. 6E).

A significant decrease of the level of inflammation at the histological level was observed in DSS-treated mice receiving the anti-AGR2 antibody administered at the dose of 10 μg/mouse by IP compared to DSS-treated mice receiving only the vehicle with respectively, a score of 2.42±0.58 vs 7.79±1.53, p=0.0064 (FIG. 6E). This corresponded to an improvement of 69% of the inflammatory lesions.

A similar improvement of the inflammation at the histological level was observed in the DSS mice receiving the anti-AGR2 at the dosage of 40 μg/mouse by IP (2.77±0.91 vs 7.79±1.53, p=0.01272) corresponding to 65% of decrease of the inflammatory lesions (FIG. 6E).

On the other hand, a trend to decrease the inflammation at the histological level was recorded in the group of DSS mice receiving the anti-AGR2 antibody administered by IP at the dosage of 5 μg/mouse (5.50±1.27 vs 7.79±1.53, ns) and 20 μg/mouse (5.21±1.29 vs 7.79±1.53, ns) (FIG. 6E).

Regarding the anti-AGR2 antibody administered by IV route, the lowest tested dose of 5 μg induced a significant decrease of the inflammation at the histological level compared to DSS mice receiving only the vehicle (mean score of 3.53±1.33 vs 7.79±1.53, p=0.047, respectively), corresponding to 55% of decrease of the inflammatory lesions (FIG. 6E). The highest tested dose of the anti-AGR2 antibody (20 μg/mouse by IV route) induced only a trend to decrease the score of inflammatory lesions at the histological level (4.23±1.51 vs 7.79±1.53, ns) (FIG. 6E).

In conclusion, the anti-AGR2 antibody was able to exert strong anti-inflammatory properties at the histological level and the IP administration of the antibody at the dosage of 10 μg/mouse exerted the highest anti-inflammatory properties.

### Myeloperoxidase Level

Myeloperoxidase (MPO) level is a quantitative marker (enzyme of polymorphonuclear neutrophil granules) used to evaluate neutrophil recruitment in tissues and indirectly colonic inflammation mediated by neutrophils. Total proteins were extracted from a restricted area of the whole colon and cytokine level expression was determined using ELISA method.

At the phase of wound-healing/beginning of the chronic colitis of inflammation occurring 7 days after the last DSS administration, a significant increase of the MPO was measured in the colon of DSS mice receiving the vehicle compared to healthy control mice receiving the vehicle (179.34±43.77 vs 16.36±2.64, p=0.02696, respectively) (FIG. 6F). The results expressed in ng of MPO/mg of total protein extracted from a restricted area of the colon, indicate that the inflammation induced by DSS was still sustained at day 12 (sacrifice). An important number of inflammatory cells infiltrate was still present in the colon of DSS-treated mice.

A significant decrease of the MPO level was recorded in DSS-treated mice receiving anti-AGR2 either by IP or IV route. Indeed, at the dose of 20 μg/mouse (IP) compared to DSS-treated mice receiving only the vehicle (mean quantity of 60.31+21.52 vs 179.34±43.77, p=0.01164 respectively) the decrease reached 66% (FIG. 6F).

A similar efficacy was recorded in the groups of DSS-treated mice receiving the anti-AGR2 by IP at the dosage of 5 μg, (with a decrease of the MPO quantity of 56.42±9.05 vs 179.34±43.77, p=0.006134, respectively) corresponding to 69% of decrease of inflammatory cells infiltrate (FIG. 6F). Regarding the IV administration a significant decrease of 60% of the quantity of MPO was recorded in DSS-treated mice vs DSS mice receiving only the vehicle (71.68±26.97 vs 179.34±43.77, p=0.02628 in ng of MPO/mg of total protein) (FIG. 6F).

This improvement of the inflammatory cells infiltrate was reaching up to 80% in the groups of DSS-treated mice receiving the anti-AGR2 at the dosage of 5 μg by IV (34.98±7.44 vs 179.34±43.77, p=0.0009017 in ng of MPO/mg of total protein) (FIG. 6F).

### Cytokines & Chemokines Levels

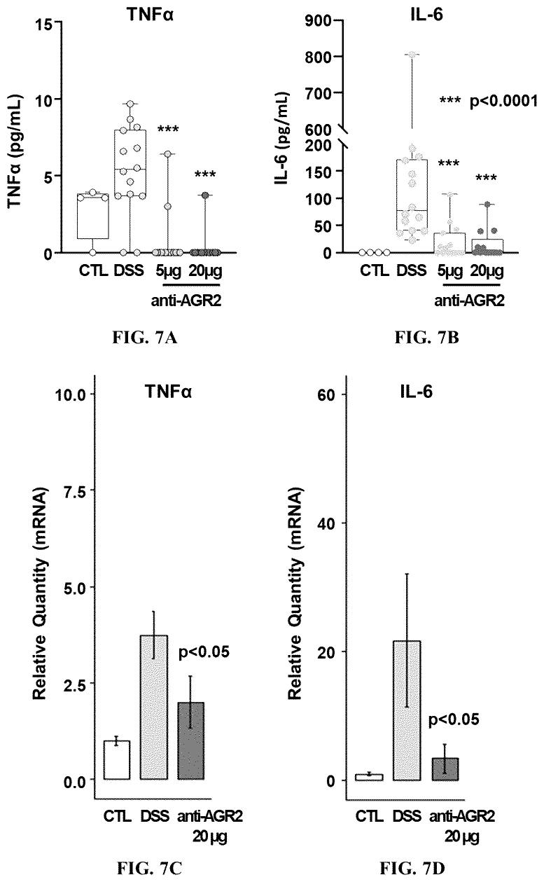

As shown on FIGS. 7A-D, blocking extracellular AGR2 (eAGR2) using an anti-AGR2 antibody of the present invention in the mouse model of acute colitis induced by DSS downregulates circulating cytokines and chemokines involved in inflammation, in particular TNFα (FIGS. 7A and 7C) and IL-6 (FIGS. 7B and 7D). This decrease of cytokines and chemokines levels is observed on TNFα and on IL-6 both at the systemic level (FIGS. 7A and 7B) and at the mRNA level (FIGS. 7C and 7D).

### Immune Infiltrate

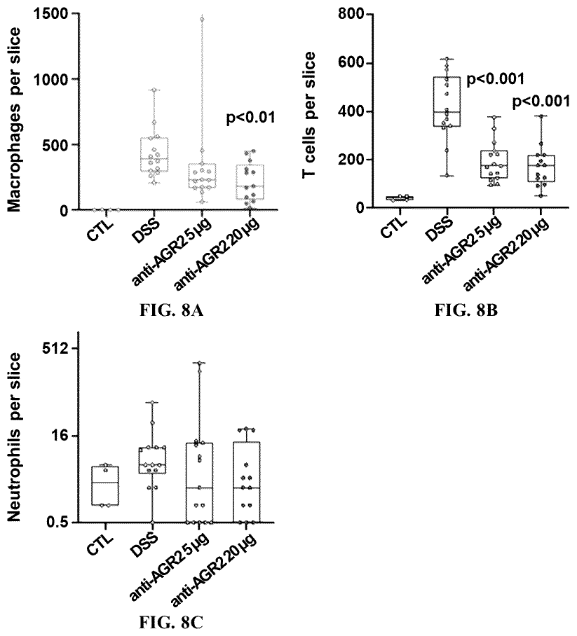

Cross-sections of colons from 46 mice were subjected to IHC staining for the presence and amount of T-cells (FIG. 8B), macrophages (FIG. 8A) and neutrophils (FIG. 8C), using digital morphometric analysis. As shown on FIG. 8B, the naive mice group (CTL) had significantly lower number of T-cells compared to the other groups, and in particular the DSS+PBS group (DSS) which exhibited significantly higher number of T-cells. Anti-inflammatory effect of anti-AGR2 antibody is evidenced by a strong decrease in immune infiltrating cells, i.e., macrophages (p<0.01) (FIG. 8A) and T cells (p<0.001) (FIG. 8B) at the two anti-AGR2 dosage (5 μg and 20 μg as indicated) by IV route.

### CONCLUSION

All together these results demonstrated the significant anti-inflammatory properties of the anti-AGR2 antibody administered by intraperitoneal or intravenous route on clinical parameters, inflammation at the histological and cellular levels.

### Example 8: In Vivo Efficacy of the Anti-AGR2 Monoclonal Antibody in Chronic DSS Model

### Material and Methods

### Animal Model and Colitis Induction

Animal experiments were performed in accredited facilities at Institut Pasteur from Lille according to governmental guidelines. All the studies were approved by the local investigational ethics review board (Nord-Pas-de-Calais CEEA No 75, Lille, France; protocol reference numbers 352012 and 19-2009R) and French government agreement no APAFIS #7542-20 17030609233680). Animals were housed five per cage and had free access to standard mouse chow and tap water.

Groups of 20 male eight-week-old C57Bl/6 mice, randomly assigned, were used. Mice were exposed to 2.5% DSS (40 kD; MP Biomedicals) for 7 days followed by 7 days of normal drinking water. The DSS exposure and recovery cycle were repeated three times (i.e., mice received DSS from day 0 to day 7, from day 14 to day 21 and from day 28 to day 35) and mice were sacrificed 7 days after the third cycle of DSS on day 42.

65 C57Bl/6 mice were allocated to the following groups, as described in Table 8 below.

TABLE 8
Groups of mice for in vivo study of DSS-induced chronic colitis.

| | | | | Number |
|---|---|---|---|---|
| Groups | Route | Dosing | Volume | of mice |
| Non-DSS control + | IP | Every 2 days from | 100 μL | 5 |
| vehicle | | day 0 to day 42 | | |
| DSS + vehicle | IP | Every 2 days from | 100 μL | 20 |
| | | day 0 to day 42 | | |
| DSS + anti-AGR2 | IP | Every 2 days from | 100 μL | 20 |
| 10 μg/mouse | | day 0 to day 42 | | |
| Preventive treatment | | | | |
| DSS + anti-AGR2 | IP | Every 2 days from | 100 μL | 20 |
| 10 μg/mouse | | day 18 to day 42 | | |
| Curative treatment | | | | |

### Antibody Treatment

TABLE 9
Sequences of the anti-AGR2 antibody used in Example 8.

| | |
|---|---|
| V_{H}-CDR1 | SEQ ID NO: 8 |
| V_{H}-CDR2 | SEQ ID NO: 10 |
| V_{H}-CDR3 | SEQ ID NO: 12 |
| V_{L}-CDR1 | SEQ ID NO: 13 |
| V_{L}-CDR2 | SEQ ID NO: 14 |
| V_{L}-CDR3 | SEQ ID NO: 15 |

The anti-AGR2 monoclonal antibody was administered at a dose of 10 μg per mouse by intraperitoneal (IP) route, every two days, either (1) starting at day 0 and until euthanasia at day 42 for the preventive treatment, or (2) starting at day 18 until euthanasia at day 42 for the curative treatment.

### Clinical Examination

### Mortality and Clinical Signs

The effects of the anti-AGR2 antibody administered by intraperitoneal route were recorded on mortality every day and on body weight evolution every two days for the duration of the study (from day 0 to day 42) and compared to the effects recorded in DSS mice receiving only the vehicle.

### Disease Activity Index (DAI)

DAI was evaluated as described in Example 7 above.

### Clinical Score: Fibrosis Score

At euthanasia, the presence of fibrotic signs (Dilatation, thickness, stenosis and adhesions) were determined. The macroscopic score of fibrosis comprises 4 different parameters of the colon: the presence of adhesion, the thickness of the colon, the dilatation of the colon and the presence of stenosis. For each of these parameters a score from 0 to 3 is attributed (0→absence, 1→mild, 2→moderate, 3→severe).

### Assessment of Histological Colonic Lesion

Assessment of the level of inflammation was performed as described above in Example 7.

Evaluation of Gene Expression by qRT-PCR

Frozen colonic samples were homogenized, and the mRNA expression of the main pro-fibrotic mediators, such as TGF-β, α-SMA, Col1A1 and Fibronectin, were assessed by quantitative RT-PCR. Briefly, total RNA was extracted with a Nucleospin RNA kit (Macherey-Nagel, Hoerdt, France). After RNAse inactivation, the total RNA was cleaned of traces of genomic DNA via a DNAse treatment and eluted in RNAse-free, DEPC-free water. The purity of the RNA was evaluated by UV spectroscopy on a Nanodrop system from 220 to 350 nm. One ug of total RNA was used to perform a quantitative RT-PCR by using LightCycler FastStart DNA Master SYBR Green I from Roche Diagnostics (Indianapolis, IN) according to the manufacturer's protocol. Sequences and relative NCBI references for the primer set are listed in Table 10 below. For each reaction, a critical threshold cycle (Ct) value, indicating the cycle number at which the DNA amplification was determined. Relative gene expression value was calculated as E=2−ΔCt, where ΔCt is the difference in crossing points between GAPDH and each gene.

TABLE 10
Sequences of primers used for the qRT-PCR

| | | | SEQ |
|---|---|---|---|
| | Primer | Primer sequences | ID |
| Mouse gene | direction | (from 5′ to 3′) | NO |
| β-actin | Forward | gggTCAgAAggATTCCTATg | 50 |
| | Reverse | ggTCTCAAACATgATCTggg | 51 |
| Fibronectin | Forward | CgAAgCCgggAAgAgCAAg | 52 |
| | Reverse | cgTTCCCACTgCTgATTTA | 53 |
| | | TCTg | |
| α-SMA | Forward | CCTgACgggCAggTgATC | 54 |
| | Reverse | ATgAAAgATggCTggAAg | 55 |
| | | AgAgTCT | |
| Col1A1 | Forward | gAgTACTggATCgACCCT | 56 |
| | | AACCAA | |
| | Reverse | ACACAggTCTgACCTgTC | 57 |
| | | TCCAT | |
| TGF β | Forward | CCCgAAgCggACTACTAT | 58 |
| | | gCT | |
| | Reverse | gTTTTCTCATAgATggCg | 59 |
| | | TTgTTg | |

### Assessment of Colonic Fibrosis

Collagen deposition was evaluated by Picrosirius red staining of serial 4 μm sections for each colon sample, according to the manufacturer's protocol. Briefly, colonic tissues were collected and fixed in fresh 4% paraformaldehyde (PFA)/PBS solution, dehydrated, and embedded in paraffin according to the standard method. Sections of 4 μm of dried colon were stained with 0.1% Direct Red stain (Sigma Aldrich)/0.5% Picric Acid (Sigma Aldrich) for 60 minutes after deparaffinization and rehydration. Slides were analyzed using the web-based Image J software. A quantitative analysis of connective tissue deposition was performed by using the threshold detection method for the grayscale image.

### Alpha-Smooth Muscle Actin (Alpha-SMA) IHC

Alpha-smooth muscle actin (alpha-SMA) is the actin isoform that predominates within vascular smooth-muscle cells and plays an important role in fibrogenesis. To detect myofibroblasts activation, colon sections were deparaffinized and rehydrated through graded alcohols to water and, after 30 min of blocking with 5% Albumine Bovine Serum. Slides were incubated with a primary monoclonal antibody α-smooth muscle actin (α-SMA) (Abcam—ab5694). Detection of α-SMA-positive cells was performed by incubating each section with a green-fluorescent Alexa-Fluor conjugated secondary antibody (AlexaFluor 488, Thermo Fisher Scientific). Irrelevant isotype-matched antibodies were used as controls. Nuclei were visualized as blue fluorescence obtained by adding 4′,6-diamidino-2-phenylindole (DAPI, Thermo Fisher Scientific) to the mounting medium. In each section, α-SMA-positive cells were counted in three randomly chosen high-power fields at a magnification of 20-fold. A quantitative analysis of the percentage of the α-SMA-positive cells was performed by using a threshold detection method for the grayscale image employing the web-based Image J software.

### Blood and Tissue Sampling

Serum and samples were collected and processed as described in Example 7 above.

### Statistical Analysis

All comparisons were analyzed using the Permutation Test for two independent samples. Statistics have been calculated using the StatXact software (Cytel Inc, Cambridge, MA, USA). Differences were considered statistically significant if the p value was <0.05.

### Results

In order to evaluate the anti-fibrotic properties of the anti-AGR2 monoclonal antibody, anti-AGR2 monoclonal antibody was administered as preventive or curative treatment by intraperitoneal route, at a dose of 10 μg/mouse, in the model of chronic colitis induced by dextran sulfate sodium (DSS) in C57Bl/6 mice.

### Mortality

Mortality in each group of mice was monitored every day. Due to the severe body weight loss induced by DSS administration and the severe colitis, mice died in the different groups of mice at the end of the first cycle of DSS. Nine mice died in the group of DSS mice receiving the vehicle, six in the group of mice receiving the anti-AGR2 antibody by IP at the dosage of 10 μg in a preventive mode and 5 mice died in the group of DSS mice receiving the anti-AGR2 antibody by IP at the dosage of 10 μg in a curative treatment mode. Death can be attributed to the severe colitis induced by DSS inducing an important body weight loss up to 20% vs their initial body weight. No more death was recorded after the end of the first cycle until euthanasia after two other additional cycles of colitis induction. It is known in this model that animals surviving the first DSS cycle are more “resistant” to subsequent DSS cycles, leading to a body weight recovery higher after each new DSS cycle.

In addition, these results indicated that the intraperitoneal administration of anti-AGR2 antibody every two days in a preventive or curative treatment mode is safe even under inflammatory conditions.

### Disease Activity Index (DAI)

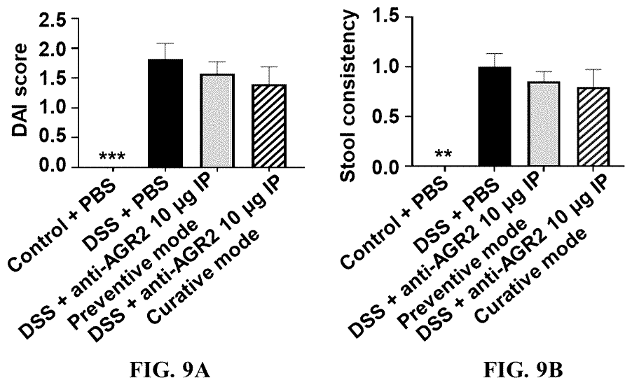

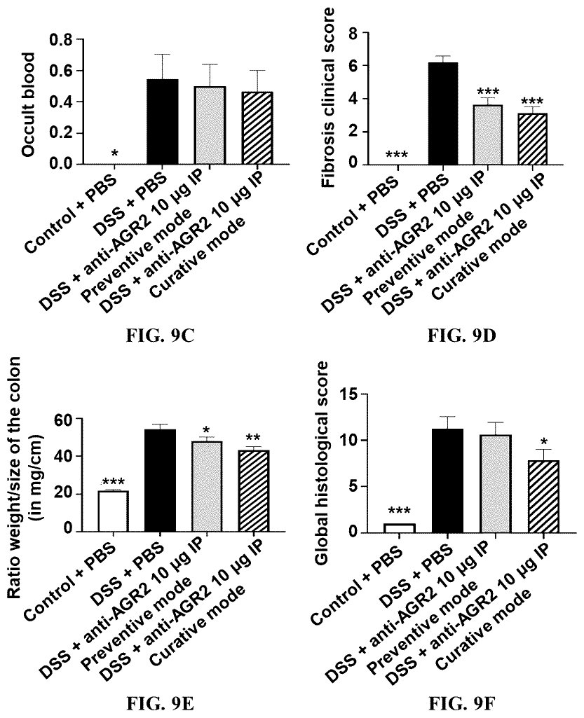

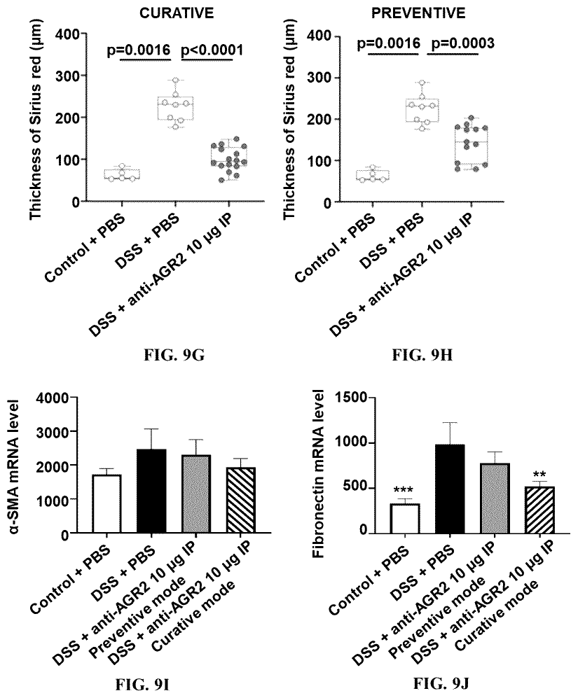

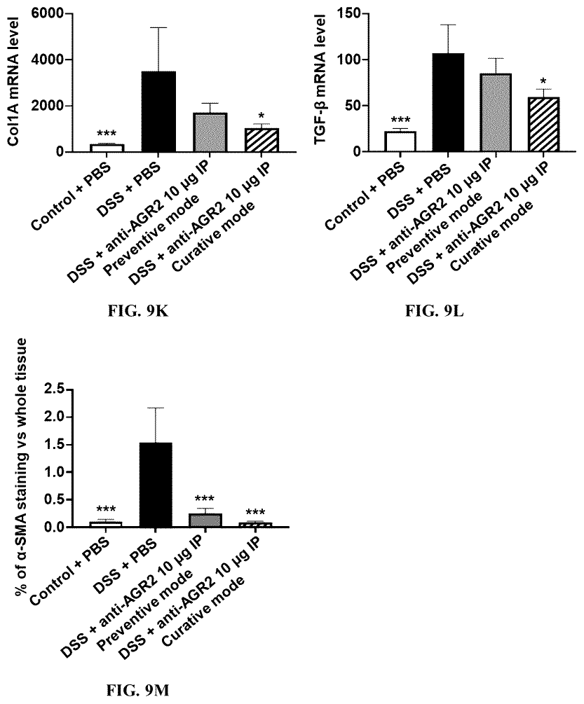

The DAI score was recorded the day of euthanasia at Day 42, 7 days after the last DSS administration of the third cycle of DSS corresponding to a wound-healing phase. The DAI score was significantly increased in the group of colitic mice receiving the vehicle compared to healthy control group (1.82±0.26 vs 0.00±0.00 respectively, p=0.00023) (FIG. 9A). This indicated that colitis remained at sustained level in the C57bl6 mice even if the inflammation was less severe after 3 cycles of DSS administration. The decrease of inflammation was due to the development of fibrosis, a direct consequence of the repeated inflammation induced by the 3 cycles of DSS.

A trend of improvement of the DAI score was observed in all the groups of DSS mice receiving the anti-AGR2 antibody intraperitoneally in a preventive or curative mode (FIG. 9A). A decrease of the clinical score of 16% for the preventive treatment and 25% for the curative treatment was observed compared to the DSS mice receiving only the vehicle (FIG. 9A). The curative treatment mode of anti-AGR2 antibody at the dosage of 10 μg/mouse exerted the highest improvement of the DAI score by decreasing by 25% the DAI score (1.40±0.30 vs 1.87±0.26, ns) 7 days after the third cycle of DSS (i.e., day 42 at euthanasia).

The improvement of the DAI score can be explained by analyzing independently the different parameters (i.e., evolution of body weight, stool consistency, and presence of blood in the feces).

### Stool Consistency

In colitic mice receiving the vehicle, a significant increase of the score of stool consistency was recorded indicating that mice suffered from a strong colitis inducing diarrhea illustrated by a score of 1.00±0.13 vs 0.00±0.00, p=0.0014 compared to healthy control mice without any sign of colitis and having normal feces consistency (score of 0) (FIG. 9B).

A similar trend to decrease the score of stool consistency was recorded in the groups of DSS mice receiving the anti-AGR2 antibody in a preventive or in a curative mode (FIG. 9B). Indeed, a decrease of around 14% was recorded in the group of DSS mice receiving the anti-AGR2 antibody in a preventive mode and 20% in the group of DSS mice having received the anti-AGR2 in a curative treatment mode (FIG. 9B).

### Presence of Blood in the Feces

At the wound-healing phase in colitic mice receiving the vehicle after 3 cycles of colitis induced by DSS, a moderate but significant increase of the score of presence of occult blood in feces was recorded indicating that mice have suffered from colitis compared to healthy control mice without any sign of colitis with respectively a score of 0.55±0.16 in DSS+vehicle vs 0.00±0.00 in control+vehicle, p=0.057 (FIG. 9C).

A trend to decrease the presence of occult blood, with a higher efficacy when anti-AGR2 antibody is administered in a curative treatment mode was observed (FIG. 9C). These results indicated that the anti-AGR2 antibody improved moderately the clinical parameters of the colitis. The lack of significant effects of the anti-AGR2 antibody could be due to the fact that in the model of chronic colitis induced by 3 cycles of DSS, the level of inflammation was lower after the third cycle of colitis induction by DSS because fibrosis started to settle in the colon of DSS mice.

### Fibrosis Clinical Score

Intestinal fibrosis is characterized by a shortening and thickening of the colon and by adherences. Measuring the weight/size ratio of the colon is also an indicator of the level of inflammation and fibrosis.

At euthanasia, before taking off the colon, the macroscopic score of fibrosis was evaluated. This score comprises 4 different parameters of the colon: the presence of adhesion, the thickness of the colon, the dilatation of the colon and the presence of stenosis. For each of these parameters a score from 0 to 3 is attributed (0=absence, 1=mild, 2=moderate, 3=severe).

The chronic inflammation induced by 3 cycles of DSS induced an important intestinal fibrosis compared to the group of healthy mice receiving only the vehicle with respectively a mean score of 6.18±0.40 vs 0.00±0.00, p=0.0002289 (FIG. 9D).

A significant decrease of the signs of fibrosis at the macroscopic level was recorded in the groups of DSS mice receiving the anti-AGR2 antibody in a preventive treatment mode compared to colitic mice receiving the vehicle with respectively a mean score of fibrosis of 3.64±0.40 vs 6.18±0.040, p=0.0003318 corresponding to a decrease of 41% of the score of fibrosis (FIG. 9D).

Similar results were observed in the group of DSS mice treated by anti-AGR2 antibody in a curative treatment mode. Indeed, a significant decrease of the macroscopic signs of fibrosis was observed compared to DSS mice receiving the vehicle with respectively a mean score of 3.13±0.40 vs 6.18±0.40, p<0.0001, corresponding to a decrease of 51% of the score of fibrosis (FIG. 9D).

### Ratio Weight/Size of the Colon

As intestinal fibrosis is characterized by a shortening and thickening of the colon and adherences, the measure of the weight/size ratio of the colon is another indicator of the level of inflammation and fibrosis.

At euthanasia, colons were carefully dissected and their size was recorded. The luminal content was then removed from the colon before weighting the colon. Indeed, a decrease in the size of the colon is induced by severe inflammation and an increase of the colon weight is also observed due to edema/inflammatory infiltrate caused by severe inflammation. In healthy mice, the mean size of the colon was 9.16±0.27 cm and the mean weight was 200.60±8.82 mg (FIG. 9E).

The ratio of the weight/size of the colon was significantly increased in the group of DSS mice receiving the vehicle when compared to the healthy control group without colitis with, respectively, a ratio weight/size of the ratio of 54.25±2.76 vs 21.87±0.49, p=0.0002289 (FIG. 9E). This result confirmed that colonic inflammation remained high 7 days after the last DSS administration in the C57BL/6 genetic background and that fibrosis was present following the repeated cycle of inflammation induced by DSS.

A significant decrease of the ratio weight/size of the colon was observed in both treated groups of mice receiving the anti-AGR2 antibody at the dosage of 10 μg/mouse either in preventive treatment mode or in curative treatment mode, compared to colitic mice receiving only the vehicle (FIG. 9E).

This result confirmed that the anti-AGR2 antibody had anti-inflammatory properties but also anti-fibrotic properties.

### Evaluation of Colonic Inflammation at the Histological Level

Evaluation of inflammation and colonic lesions at histological level was performed according to a validated score for DSS-induced colitis. Results are expressed as a mean±SEM score. As described above, May-Grunwald-Giemsa-stained sections of colonic tissue were evaluated for inflammation using a multiparametric scoring system (0-18). The system assessed the severity (0-1) and extent of inflammation (0-3), the regeneration level (0-4), crypt damage (0-4), and the percent involvement (extension) (1-4).

Seven days after the last DSS administration of the third cycle of DSS, a sustained and significant colonic inflammation was still recorded at the histological level (FIG. 9F) in the groups of DSS mice receiving the vehicle compared to the healthy control group without colitis (11.27±1.28 vs 1.00±0.00 p=0.0002289).

A significant decrease of the level of inflammation at the histological level was observed in colitic mice receiving the anti-AGR2 antibody administered at the dosage of 10 μg/mouse in a curative treatment mode compared to colitic mice receiving only the vehicle (score of 7.87±1.16 vs 11.27±1.28 respectively, p=0.0035), corresponding to an improvement of 30% of the inflammatory lesions (FIG. 9F).

No improvement of the inflammation at the histological level was observed in the DSS mice receiving the anti-AGR2 at the dosage of 10 μg/mouse in a preventive treatment mode (10.64±1.16 vs 11.27±1.28) (FIG. 9F).

In conclusion, the anti-AGR2 antibody administered at the dosage of 10 μg/mouse by IP in a curative mode exerted strong anti-inflammatory properties at the histological level.

### Histological Evaluation of Collagen Deposition

Deposition of collagen fibers was evaluated on mice colons by the Picrosirius Red histological staining. Quantification of collagen deposition was performed using the web-based Image J software. Three different fields for each section were analyzed to measure collagen deposition. Data are expressed as mean±sem.

Collagen deposition level was significantly increased in the group of colitic mice receiving the vehicle after 3 cycles of colitis induction by DSS compared to their respective healthy control group with, respectively, 226.38±10.67 vs 62.80±4.18, p=0.0016 (FIGS. 9G and 9H).

A significant decrease of the level of collagen deposition was observed in colitic mice receiving preventively the anti-AGR2 with a higher efficacy compared to the administration in a curative mode (100.21±5.99 vs 226.38±10.67, p<0.0001) corresponding to 56% of improvement of the fibrosis (FIGS. 9G and 9H).

Indeed, a significant decrease of the level of collagen deposition in the colon was also recorded in colitic mice receiving curatively the anti-AGR2 (mean level of 142.87±8.40 vs 226.38±10.67, p=0.0003) corresponding to a decrease of 37% of the deposition of collagen (FIG. 9G).

### Evaluation of Fibrotic Markers Gene Expression

Evaluation of gene expression of fibrosis markers (TGF-β, α-SMA, Fibronectin and Collagen I expression) was performed using qRT-PCR in colonic sections of mice. Data are expressed as mean±SEM.

Regarding α-SMA gene expression, a trend to increase the level of this gene was observed in DSS mice receiving vehicle compared to control healthy mice receiving the vehicle without any significance (mean level 2473.72±590.81 vs 1713.67±185.85 respectively, ns). No effect of the administration of anti-AGR2 in a preventive or curative treatment mode was recorded regarding this marker of fibrosis at the mRNA level

(FIG. 9I).

Regarding the 3 other studied fibrotic markers, i.e., Col1A, TGF-β and fibronectin, a significant increase of the gene expression level at the mRNA level was observed in the colon of colitic mice receiving the vehicle compared to healthy control mice after 3 cycles of DSS (FIGS. 9J, 9K and 9L).

Indeed, a significant increase of the level of expression was recorded in colitic mice vs healthy mice with respectively a mean level of 3509.06+1895.68 vs 333.42+45.08, p=0.0002289 for COL1A (FIG. 9K), 107.25±30.76 vs 22.71±2.73, p=0.0002289 for TGF-β (FIG. 9L) and 986.39±238.86 vs 333.54±50.60, p=0.0006868 for fibronectin (FIG. 9J).

No significant effect on the 4 studied fibrotic markers gene expression was measured in DSS mice receiving preventively the anti-AGR2 antibody compared to DSS mice receiving only the vehicle (FIGS. 9I to 9L).

On the other hand, a significant decrease of the level of gene expression of COL1A (FIG. 9K) (1041.50±176.83 vs 3509.06±1895.68, p=0.02367), TGF-β (FIG. 9K) (59.61±8.60 vs 107.25±30.76, p=0.0397) and Fibronectin (FIG. 9J) (519.05±57.47 vs 986.39±238.86, p=0.009682) was recorded in the colon of DSS mice receiving curatively the anti-AGR2 antibody.

These results confirmed that prolonged inflammation induced fibrosis and increased the level of expression of fibrotic markers. The anti-AGR2 antibody administered at the dosage of 10 μg by IP in a curative treatment mode was able to exert anti-fibrotic properties in the colon of DSS-mice.

### Evaluation of α-SMA Protein Expression by IHC Staining

Fibrosis is characterized by a proliferation of myofibroblasts, and α-SMA is a specific marker of activation of those cells. The expression of α-SMA in the different groups of treated mice was evaluated at the protein level by immunohistochemistry.

In the model of chronic colitis induced by 3 cycles of DSS, a significant increase of α-SMA at the protein level was measured in the colon of DSS mice receiving the vehicle compared to control healthy mice receiving the vehicle with respectively a mean level of 1.54±0.62 vs 0.10±0.04, p=0.0002289 (FIG. 9M).

A significant decrease of α-SMA protein level was recorded in the group of DSS mice receiving the anti-AGR2 antibody administered in a preventive treatment mode compared to colitic mice receiving the vehicle with respectively a mean quantity of 0.25±0.09 vs 1.54±0.62, p=0.0005981 corresponding to a decrease of 81% of the presence of the fibrotic marker (FIG. 9M).

A higher efficacy to decrease α-SMA at the protein level was observed in the colon of colitic mice receiving the anti-AGR2 antibody in a curative treatment mode compared to colitic mice receiving the vehicle reaching up to 99% of improvement with respectively a mean quantity of α-SMA of 0.09±0.02 vs 1.54±0.62, p<0.0001 (FIG. 9M).

This result confirmed that the anti-AGR2 antibody exerted strong anti-fibrotic properties and that the curative treatment mode exerted higher anti-fibrotic effects at the dosage of 10 μg in the model of chronic colitis induced by 3 cycles of DSS in C57bl6 mice.

### CONCLUSION

Efficacy was documented on clinical parameters, inflammation, fibrosis, histological parameters and at the molecular level at day 42, corresponding to fibrotic phase of the model.

All together these results demonstrated the important and significant anti-inflammatory, wound-healing and anti-fibrotic properties of the anti-AGR2 antibody in a curative treatment mode.

1-15. (canceled)

16. An isolated antibody, or binding fragment thereof, that specifically binds to Anterior Gradient 2 protein (AGR2), wherein said antibody, or binding fragment thereof, comprises:
a heavy chain variable region (VH) comprising
the following three complementary-
determining regions (CDRs):
CDR1:
(SEQ ID NO: 8)
RSWMN;
CDR2:
(SEQ ID NO: 9)
WIYPGDGDTNYNGKXKD
wherein X is F or V;
CDR3:
(SEQ ID NO: 12)
GGYDGSPWLSY;
and
a light chain variable region (VL)
comprising the following three CDRs:
CDR1:
(SEQ ID NO: 13)
KASQDINSYLS;
CDR2:
(SEQ ID NO: 14)
RANRLVD;
and
CDR3:
(SEQ ID NO: 15)
LQYDEFPFT.

17. The isolated antibody, or binding fragment thereof, according to claim 16, wherein said antibody, or binding fragment thereof, comprises:
a heavy chain variable region (VH) comprising framework regions sharing at least 80% sequence identity with the framework regions of SEQ ID NO: 16; and a light chain variable region (VL) comprising framework regions sharing at least 80% sequence identity with the framework regions of SEQ ID NO: 19; or
a heavy chain variable region (VH) comprising framework regions sharing at least 80% sequence identity with the framework regions of SEQ ID NO: 17; and a light chain variable region (VL) comprising framework regions sharing at least 80% sequence identity with the framework regions of SEQ ID NO: 19; or
a heavy chain variable region (VH) comprising framework regions sharing at least 80% sequence identity with the framework regions of SEQ ID NO: 16; and a light chain variable region (VL) comprising framework regions sharing at least 80% sequence identity with framework regions of SEQ ID NO: 20; or
a heavy chain variable region (VH) comprising framework regions sharing at least 80% sequence identity with the framework regions of SEQ ID NO: 17; and a light chain variable region (VL) comprising framework regions sharing at least 80% sequence identity with the framework regions of SEQ ID NO: 20; or
a heavy chain variable region (VH) comprising framework regions sharing at least 80% sequence identity with the framework regions of SEQ ID NO: 18; and a light chain variable region (VL) comprising framework regions sharing at least 80% sequence identity with the framework regions of SEQ ID NO: 21.

18. The isolated antibody, or binding fragment thereof, according to claim 16, wherein said antibody, or binding fragment thereof, comprises:
a heavy chain variable region (VH) having a sequence consisting of sequence SEQ ID NO: 16; and a light chain variable region (VL) having a sequence consisting of sequence SEQ ID NO: 19; or
a heavy chain variable region (VH) having a sequence consisting of sequence SEQ ID NO: 17; and a light chain variable region (VL) having a sequence consisting of sequence SEQ ID NO: 19; or
a heavy chain variable region (VH) having a sequence consisting of sequence SEQ ID NO: 16; and a light chain variable region (VL) having a sequence consisting of sequence SEQ ID NO: 20; or
a heavy chain variable region (VH) having a sequence consisting of sequence SEQ ID NO: 17; and a light chain variable region (VL) having a sequence consisting of sequence SEQ ID NO: 20; or
a heavy chain variable region (VH) having a sequence consisting of sequence SEQ ID NO: 18; and a light chain variable region (VL) having a sequence consisting of sequence SEQ ID NO: 21.

19. The isolated antibody, or binding fragment thereof, according to claim 16, wherein said antibody, or binding fragment thereof, is an immunoconjugate.

20. A nucleic acid encoding the antibody, or binding fragment thereof, according to claim 16.

21. An expression vector comprising the nucleic acid according to claim 20.

22. A cell comprising the nucleic acid according to claim 20 or an expression vector comprising said nucleic acid.

23. A pharmaceutical composition comprising the isolated antibody, or binding fragment thereof, according to claim 16, an nucleic acid encoding said antibody, or binding fragment thereof, an expression vector comprising a nucleic acid encoding said antibody, or binding fragment thereof, or a cell comprising a nucleic acid encoding said antibody, or binding fragment thereof, or comprising an expression vector comprising said nucleic acid, and at least one pharmaceutically acceptable excipient.

24. A method of treating a subject in need thereof, said method comprising administering to said subject the isolated antibody, or binding fragment thereof, according to claim 16, a nucleic acid encoding said antibody, or binding fragment thereof, an expression vector comprising a nucleic acid encoding said antibody, or binding fragment thereof, a cell comprising a nucleic acid encoding said antibody, or binding fragment thereof, or comprising an expression vector comprising said nucleic acid, or a pharmaceutical composition comprising said isolated antibody, or binding fragment thereof, said nucleic acid, said expression vector or said cell, and at least one pharmaceutically acceptable excipient.

25. A method of treating a mucosal inflammatory disease, or a cancer, in a subject in need thereof, said method comprising administering to said subject the isolated antibody, or binding fragment thereof, according to claim 16, a nucleic acid encoding said antibody, or binding fragment thereof, an expression vector comprising a nucleic acid encoding said antibody, or binding fragment thereof, a cell comprising a nucleic acid encoding said antibody, or binding fragment thereof, or comprising an expression vector comprising said nucleic acid, or a pharmaceutical composition comprising said isolated antibody, or binding fragment thereof, said nucleic acid, said expression vector or said cell, and at least one pharmaceutically acceptable excipient.

26. The method according to claim 25, wherein said isolated antibody, or binding fragment thereof, neutralizes the pro-inflammatory activity of eAGR2 and/or the pro-fibrotic activity of eAGR2.

27. The method according to claim 25, wherein the mucosal inflammatory disease is selected from the group consisting of Crohn's disease, ulcerative colitis, primary sclerosing cholangitis, chronic pancreatitis, microscopic colitis, inflammatory bowel disease (IBD), endometriosis, appendicitis, inflammatory bowel syndrome, idiopathic pulmonary fibrosis, systemic sclerosis, systemic sclerosis associated with interstitial lung disease, asthma and chronic obstructive pulmonary disease.

28. The method according to claim 25, wherein said cancer is selected from the group consisting of colon cancer, gastrointestinal cancer, prostate cancer, pancreatic cancer, oral cancer, breast cancer, lung cancer, ovarian cancer, thyroid cancer, cholangiocarcinoma, head and neck squamous cell carcinoma, brain glioblastoma, adrenocortical carcinoma, bladder cancer, kidney cancer, penile cancer, renal cancer, testicular cancer, urethral cancer, colorectal cancer, cervical cancer, uterine cancer, endometrial cancer, vaginal cancer, vulvar cancer, gestational trophoblastic disease (GTD), and primary peritoneal cancer.

29. An in vitro method for detecting or quantifying AGR2 expression in a biological sample, comprising contacting said biological sample with the isolated antibody, or binding fragment thereof, according to claim 16.

30. The in vitro method according to claim 29, wherein said method is for diagnosing or monitoring an AGR2-related disease in a subject, or for selecting a subject suffering from an AGR2-related disease for treatment targeting said disease.


SEQUENCE LISTING (59 sequences)

SEQ ID NO: 1 - protein, 175 aa, Homo sapiens
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 2 - protein, 175 aa, Mus musculus
MEKFSVSAILLLVAISGTLAKDTTVKSGAKKDPKDSRPKLPQTLSRGWGDQLIWTQTYEEALYRSKTSNRPLMVIHHLDECPHSQALKKVFAEHKEIQKLAEQFVLLNLVYETTDKHLSPDGQYVPRIVFVDPSLTVRADITGRYSNRLYAYEPSDTALLYDNMKKALKLLKTEL

SEQ ID NO: 3 - protein, 175 aa, Macaca mulatta
MEKISVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRILFVDPSLTVRADITGRYSNRLYAYEPADTALLLDNMKKALKLLKTEL

SEQ ID NO: 4 - protein, 175 aa, Pan troglodytes
MEKISVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 5 - protein, 175 aa, Rattus norvegicus
MEKFSVSAILLLVAISGTLAKDTTVKSGSKKDPKDSRPKLPQTLSRGWGDQLIWTQTYEEALYKSKTSNRPLMVIHHLDECPHSQALKKVFAENKEIQKLAEQFVLLNLIYETTDKHLSPDGQYVPRIVFVDPSLTVRADITGRYSNRLYAYEPSDTALLHDNMKKALKLLKTEL

SEQ ID NO: 6 - protein, 175 aa, Canis lupus
MEKISVSAFLLLVALSYTLAKDITVKSGAKKDTKDSAPKLPQTLSRGWGDQLIWTQTYEEALYKSKTSNKPLMIIHHLDECPHSQALKKVFAENKEIQKLAEQFVLLNLVYETTDKHLSPDGQYVPRIIFVDPSLTVRADITGRYSNRLYAYEPSDIALLLDNMKKALKLLKTEL

SEQ ID NO: 7 - protein, 175 aa, synthetic construct
MEKIPVSAFLLLVALSYTLARDTTVKPGAKKDTKDSRPKLPQTLSRGWGDQLIWTQTYEAALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 8 - protein, 5 aa, synthetic construct
RSWMN

SEQ ID NO: 9 - protein, 17 aa, synthetic construct
  VARIANT 15: note = X is F or V
WIYPGDGDTNYNGKXKD

SEQ ID NO: 10 - protein, 17 aa, synthetic construct
WIYPGDGDTNYNGKFKD

SEQ ID NO: 11 - protein, 17 aa, synthetic construct
WIYPGDGDTNYNGKVKD

SEQ ID NO: 12 - protein, 11 aa, synthetic construct
GGYDGSPWLSY

SEQ ID NO: 13 - protein, 11 aa, synthetic construct
KASQDINSYLS

SEQ ID NO: 14 - protein, 7 aa, synthetic construct
RANRLVD

SEQ ID NO: 15 - protein, 9 aa, synthetic construct
LQYDEFPFT

SEQ ID NO: 16 - protein, 120 aa, synthetic construct
QVQLVQSGAEVKKPGASVKVSCKTSGYVFSRSWMNWVRQAPGQGLEWIGWIYPGDGDTNYNGKFKDRVTLTADKSTSTVYMELSSLRSEDTAVYFCARGGYDGSPWLSYWGQGTLVTVSS

SEQ ID NO: 17 - protein, 120 aa, synthetic construct
QVQLVQSGSELKKPGASVKVSCKTSGYVFSRSWMNWVRQAPGQGLEWIGWIYPGDGDTNYNGKFKDRFVLSADKSVSTVYLQISSLKAEDTAVYFCARGGYDGSPWLSYWGQGTLVTVSS

SEQ ID NO: 18 - protein, 120 aa, synthetic construct
QVQLLESGGGLVKPGGSLRLSCATSGYVFSRSWMNWIRQAPGQGLEWIGWIYPGDGDTNYNGKVKDRFTLSADKAKNSVYLQMNSLRAEDTAVYFCARGGYDGSPWLSYWGQGTLVTVSS

SEQ ID NO: 19 - protein, 107 aa, synthetic construct
DIQMTQSPSSLSASVGDRVTITCKASQDINSYLSWFQQKPGKAPKTLIYRANRLVDGVPSRFSGSGSGQDFTLTISSLQPEDIATYYCLQYDEFPFTFGGGTKVEIK

SEQ ID NO: 20 - protein, 107 aa, synthetic construct
EIVLTQSPATLSLSPGERATLSCKASQDINSYLSWFQQKPGQAPRTLIYRANRLVDGIPARFSGSGSGQDFTLTISSLEPEDFAVYYCLQYDEFPFTFGGGTKVEIK

SEQ ID NO: 21 - protein, 107 aa, synthetic construct
EIVLTQSPDFQSVTPKEKVTITCKASQDINSYLSWFQQKPDQSPKTLIYRANRLVDGVPSRFSGSGSGQDFTLTINSLEAEDAATYYCLQYDEFPFTFGGGTKVEIK

SEQ ID NO: 22 - protein, 330 aa, Homo sapiens [sequence omitted]

SEQ ID NO: 23 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 107 aa, Homo sapiens [sequence omitted]

SEQ ID NO: 27 - protein, 450 aa, synthetic construct
QVQLVQSGAEVKKPGASVKVSCKTSGYVFSRSWMNWVRQAPGQGLEWIGWIYPGDGDTNYNGKFKDRVTLTADKSTSTVYMELSSLRSEDTAVYFCARGGYDGSPWLSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 450 aa, synthetic construct
QVQLVQSGSELKKPGASVKVSCKTSGYVFSRSWMNWVRQAPGQGLEWIGWIYPGDGDTNYNGKFKDRFVLSADKSVSTVYLQISSLKAEDTAVYFCARGGYDGSPWLSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 450 aa, synthetic construct
QVQLLESGGGLVKPGGSLRLSCATSGYVFSRSWMNWIRQAPGQGLEWIGWIYPGDGDTNYNGKVKDRFTLSADKAKNSVYLQMNSLRAEDTAVYFCARGGYDGSPWLSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 450 aa, synthetic construct
QVQLVQSGAEVKKPGASVKVSCKTSGYVFSRSWMNWVRQAPGQGLEWIGWIYPGDGDTNYNGKFKDRVTLTADKSTSTVYMELSSLRSEDTAVYFCARGGYDGSPWLSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 450 aa, synthetic construct
QVQLVQSGSELKKPGASVKVSCKTSGYVFSRSWMNWVRQAPGQGLEWIGWIYPGDGDTNYNGKFKDRFVLSADKSVSTVYLQISSLKAEDTAVYFCARGGYDGSPWLSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 450 aa, synthetic construct
QVQLLESGGGLVKPGGSLRLSCATSGYVFSRSWMNWIRQAPGQGLEWIGWIYPGDGDTNYNGKVKDRFTLSADKAKNSVYLQMNSLRAEDTAVYFCARGGYDGSPWLSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, synthetic construct
DIQMTQSPSSLSASVGDRVTITCKASQDINSYLSWFQQKPGKAPKTLIYRANRLVDGVPSRFSGSGSGQDFTLTISSLQPEDIATYYCLQYDEFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, synthetic construct
EIVLTQSPATLSLSPGERATLSCKASQDINSYLSWFQQKPGQAPRTLIYRANRLVDGIPARFSGSGSGQDFTLTISSLEPEDFAVYYCLQYDEFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 214 aa, synthetic construct
EIVLTQSPDFQSVTPKEKVTITCKASQDINSYLSWFQQKPDQSPKTLIYRANRLVDGVPSRFSGSGSGQDFTLTINSLEAEDAATYYCLQYDEFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 1410 bp, synthetic construct
  source 1..1410: mol_type = other DNA
atgggctggtcctgcatcatcctgttcctggtggctaccgccaccggcgtgcactcccaagtgcaactagtgcaaagtggtgcagaagtcaagaagcctggcgcttccgtgaaagtgtcctgcaagaccagcggatacgtgttctcccggtcctggatgaactgggtgcggcaggctcctggacagggcctggagtggatcggctggatctaccccggcgatggcgacaccaactacaacggcaagttcaaggacagagtgaccctgaccgccgacaagtccacctctaccgtgtacatggaactgtcttctctgagatccgaggatacagccgtgtatttttgtgccagaggcggctacgacggctctccttggctgtcctactggggccagggcacactggtgaccgtgtctagcgcaagtactaagggtccaagtgtgtttccactggctccctctagcaagtccacctctggcggcaccgccgccctgggctgcctggtcaaggactacttccccgagcccgtcaccgtgtcctggaactccggcgctctgacctccggcgtgcataccttccctgctgtgctgcagtcttctggcctgtactccctgtcctccgtcgtgacagtgccttctagctctctcggcacacagacatacatctgcaatgtgaaccacaagccttccaacaccaaagtggataagaaggtggaacctaagtcctgcgacaaaacccacacctgtcctccttgccctgctcctgaagccgctggcggaccttctgtgtttctgtttcccccaaagcccaaagacaccctgatgatctctcggacacctgaggtgacctgcgtggtggtggatgtgtctcacgaggaccccgaagtgaagttcaactggtacgtggacggcgtggaagtgcacaacgccaagaccaagccacgcgaagagcagtacaactctacctacagagtggtgtccgtgctcacagtgctgcaccaggactggctgaacggaaaagagtacaagtgcaaggtctctaacaaggctctgccagcccctatcgagaagaccatctccaaggccaagggccaacctagagagcctcaagtgtacaccctgcctcctagccgggacgagctgaccaagaaccaggtttccctgacctgtctggtgaaaggcttctacccctccgatatcgccgtggaatgggagtccaacggccagcctgagaacaactacaagaccacccctcctgtgctggactccgatggatccttcttcctgtatagcaagctgaccgtggacaagtccagatggcagcagggcaacgtgttcagctgttccgtgatgcacgaggccctgcataatcactacacccagaagtctctgagcctgtctcctggcaagtga

SEQ ID NO: 37 - DNA, 702 bp, synthetic construct
  source 1..702: mol_type = other DNA
atgggctggtcctgcatcatcctgttcctggtggctaccgccaccggcgtgcactccgatatccaaatgactcaaagtccaagtagtctctctgcctccgtgggcgacagagtgacaatcacctgtaaggcctcccaagatatcaactcctatctgtcctggttccagcagaagcctggcaaggctcctaagaccctgatctacagagccaaccggctggtcgatggcgtgcccagccggttctccggctctggatctggccaggacttcacactgaccatctccagcctgcagcctgaggacatcgccacctactactgcctgcagtacgacgagttcccatttaccttcggcggcggcaccaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 38 - DNA, 1410 bp, synthetic construct
  source 1..1410: mol_type = other DNA
atgggctggtcctgcatcatcctgttcctggtggctaccgccaccggcgtgcactcccaagtgcaactagtgcaaagtggtagtgaactgaaaaagcctggagcttctgtgaaggtgtcctgcaagacatctggctacgtgttctccagatcttggatgaactgggtgcggcaggctcctggccagggcctggaatggatcggctggatctaccccggagatggcgataccaactacaacggcaagttcaaggaccggttcgtgctgtctgccgacaagtccgtgtccaccgtgtacctgcagatctcctccctcaaggccgaggacaccgccgtgtatttttgtgccagaggcggctacgacggctctccttggctgtcctactggggccaaggcaccctggtgaccgtcagcagcgcaagtactaagggtccaagtgtgtttccactggctccctctagcaagtccacctctggcggcaccgccgccctgggctgcctggtcaaggactacttccccgagcccgtcaccgtgtcctggaactccggcgctctgacctccggcgtgcataccttccctgctgtgctgcagtcttctggcctgtactccctgtcctccgtcgtgacagtgccttctagctctctcggcacacagacatacatctgcaatgtgaaccacaagccttccaacaccaaagtggataagaaggtggaacctaagtcctgcgacaaaacccacacctgtcctccttgccctgctcctgaagccgctggcggaccttctgtgtttctgtttcccccaaagcccaaagacaccctgatgatctctcggacacctgaggtgacctgcgtggtggtggatgtgtctcacgaggaccccgaagtgaagttcaactggtacgtggacggcgtggaagtgcacaacgccaagaccaagccacgcgaagagcagtacaactctacctacagagtggtgtccgtgctcacagtgctgcaccaggactggctgaacggaaaagagtacaagtgcaaggtctctaacaaggctctgccagcccctatcgagaagaccatctccaaggccaagggccaacctagagagcctcaagtgtacaccctgcctcctagccgggacgagctgaccaagaaccaggtttccctgacctgtctggtgaaaggcttctacccctccgatatcgccgtggaatgggagtccaacggccagcctgagaacaactacaagaccacccctcctgtgctggactccgatggatccttcttcctgtatagcaagctgaccgtggacaagtccagatggcagcagggcaacgtgttcagctgttccgtgatgcacgaggccctgcataatcactacacccagaagtctctgagcctgtctcctggcaagtga

SEQ ID NO: 39 - DNA, 702 bp, synthetic construct
  source 1..702: mol_type = other DNA
atgggctggtcctgcatcatcctgttcctggtggctaccgccaccggcgtgcactccgatatccaaatgactcaaagtccaagtagtctgtctgccagcgtcggcgatagagtgacaatcacctgtaaggcctcccaagatatcaactcctatctgtcctggttccagcagaagcctggcaaggctcctaagaccctgatctacagagccaaccggctggtggacggcgtgccatctcggttctccggatctggctccggccaggacttcacactgaccatctccagcctgcagcctgaggacatcgccacctactactgcctgcagtacgacgagttcccctttaccttcggcggaggcaccaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 40 - DNA, 1410 bp, synthetic construct
  source 1..1410: mol_type = other DNA
atgggctggtcctgcatcatcctgttcctggtggctaccgccaccggcgtgcactcccaagtgcaactagtgcaaagtggtgcagaagtcaagaagcctggcgcttccgtgaaagtgtcctgcaagacctctggctacgtgttctcccggtcctggatgaactgggtgcggcaggcccctggacagggcctggagtggatcggctggatctaccccggcgatggcgacaccaactacaacggcaagttcaaggacagagtgacactgaccgccgacaagtccacatctaccgtgtacatggaactgtctagcctgagatccgaggataccgctgtgtatttttgtgccagaggcggatacgacggctctccttggctgtcctactggggccagggcaccctggtgaccgtgtctagcgcaagtactaagggtccaagtgtgtttccactggctccctctagcaagtccacctctggcggcaccgccgccctgggctgcctggtcaaggactacttccccgagcccgtcaccgtgtcctggaactccggcgctctgacctccggcgtgcataccttccctgctgtgctgcagtcttctggcctgtactccctgtcctccgtcgtgacagtgccttctagctctctcggcacacagacatacatctgcaatgtgaaccacaagccttccaacaccaaagtggataagaaggtggaacctaagtcctgcgacaaaacccacacctgtcctccttgccctgctcctgaagccgctggcggaccttctgtgtttctgtttcccccaaagcccaaagacaccctgatgatctctcggacacctgaggtgacctgcgtggtggtggatgtgtctcacgaggaccccgaagtgaagttcaactggtacgtggacggcgtggaagtgcacaacgccaagaccaagccacgcgaagagcagtacaactctacctacagagtggtgtccgtgctcacagtgctgcaccaggactggctgaacggaaaagagtacaagtgcaaggtctctaacaaggctctgccagcccctatcgagaagaccatctccaaggccaagggccaacctagagagcctcaagtgtacaccctgcctcctagccgggacgagctgaccaagaaccaggtttccctgacctgtctggtgaaaggcttctacccctccgatatcgccgtggaatgggagtccaacggccagcctgagaacaactacaagaccacccctcctgtgctggactccgatggatccttcttcctgtatagcaagctgaccgtggacaagtccagatggcagcagggcaacgtgttcagctgttccgtgatgcacgaggccctgcataatcactacacccagaagtctctgagcctgtctcctggcaagtga

SEQ ID NO: 41 - DNA, 702 bp, synthetic construct
  source 1..702: mol_type = other DNA
atgggctggtcctgcatcatcctgttcctggtggctaccgccaccggcgtgcactccgaaatcgtgctaactcaaagtccagcaactctcagcctgtctccaggcgagcgggctacactgagctgcaaggcctctcaggacatcaactcctacctgtcctggttccagcagaaacctggccaggctcctagaaccctgatctacagagccaaccggctggtggacggcatccctgccagattctccggctctggatctggccaagattttaccctgaccatctcctccctggaacctgaggacttcgccgtgtactattgtctgcagtacgacgagttccccttcaccttcggcggaggcaccaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 42 - DNA, 1410 bp, synthetic construct
  source 1..1410: mol_type = other DNA
atgggctggtcctgcatcatcctgtttctggtggccaccgctaccggagtgcactcccaagtgcaactagtgcaaagtggtgcagaagtcaagaagcctggcgcttccgtgaaagtgtcctgcaagacctccggctacgtgttctcccggtcttggatgaactgggtgcggcaggcccctggacagggcctggagtggatcggctggatctaccccggagatggcgataccaactacaacggcaagttcaaggacagagtgacactgaccgctgacaagtccacctctaccgtgtacatggaactgtctagcctgagatccgaggacaccgccgtgtatttttgtgccagaggcggctacgacggctctccttggctgtcctactggggccagggcaccctggtgacagtgtctagcgcctctacaaagggcccatctgtgtttcctctggccccttcttctaagtctacctccggcggaacagccgctctgggctgtctggtcaaggactacttccctgagcccgtgaccgtgtcctggaactctggcgctctgacctctggagtgcataccttccccgctgtgctgcagtcctctggcctgtactctctgtctagcgtcgtgaccgtgccttcttccagcctgggcacccagacctacatctgcaacgtgaaccacaagccttccaacaccaaagtggataagaaggtggaacctaagagctgcgacaagacacacacctgccctccttgtcctgctcctgaactgctgggcggccctagcgtgttcctgttcccacctaagcccaaggacaccctgatgatctctcggacccctgaggtcacctgtgtggtggtggacgtgtctcacgaggaccccgaggtgaagttcaactggtacgtggatggcgtggaagtgcacaacgccaagaccaagcctcgggaagagcagtacaactccacctacagagtggtctctgtcctgaccgtgctgcaccaggactggctgaacggcaaagagtacaagtgcaaggtgtccaacaaggccctgcctgcccctatcgagaagaccatctccaaggctaagggccagcctagagagcctcaagtgtacaccctgcctccaagccgggacgagctgaccaagaaccaggtgtctctgacctgcctggtgaaaggcttctacccctccgacatcgccgtggaatgggagtccaatggccaacccgagaacaactacaagaccacccctcctgtgctggactccgacggctccttcttcctgtactccaagctgacagtggacaagtccagatggcagcagggcaacgtgttctcctgctccgtgatgcacgaggccctgcacaatcactacacccagaagtccctgtccctctctcctggcaagtga

SEQ ID NO: 43 - DNA, 702 bp, synthetic construct
  source 1..702: mol_type = other DNA
atgggctggtcctgcatcatcctgttcctggtggctaccgccaccggcgtgcactccgaaatcgtgctaactcaaagtccagcaactctctctctgtctcctggcgagcgggctaccctgtcctgcaaggcctctcaggacatcaactcctacctgtcctggttccagcagaaaccaggacaagctcctagaaccctgatctacagagccaaccggctggtggacggcatccctgccagattctccggcagcggctctggccaggattttacactgaccatctccagcctggaacctgaggacttcgccgtgtactattgtctgcagtacgacgagttccccttcaccttcggcggaggcaccaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 44 - DNA, 1410 bp, synthetic construct
  source 1..1410: mol_type = other DNA
atgggctggtcctgcatcatcctgttcctggtggctaccgccaccggcgtgcactcccaagtgcaactagtgcaaagtggtagtgaactcaaaaagcctggcgcctctgtgaaggtgtcctgcaagacctctggctacgtgttctccagatcttggatgaactgggtgcggcaggctcctggacaaggcctggaatggatcggctggatctaccccggcgatggagataccaactacaacggcaagttcaaggaccggttcgtgctgtctgccgacaagtccgtgtccaccgtgtacctgcagatctcctccctgaaggccgaggacacagctgtctatttttgtgccagaggcggctacgacggctctccttggctgtcctactggggccagggcaccctggtgaccgttagcagcgcaagtactaagggtccaagtgtgtttccactggctccctctagcaagtccacctctggcggcaccgccgccctgggctgcctggtcaaggactacttccccgagcccgtcaccgtgtcctggaactccggcgctctgacctccggcgtgcataccttccctgctgtgctgcagtcttctggcctgtactccctgtcctccgtcgtgacagtgccttctagctctctcggcacacagacatacatctgcaatgtgaaccacaagccttccaacaccaaagtggataagaaggtggaacctaagtcctgcgacaaaacccacacctgtcctccttgccctgctcctgaagccgctggcggaccttctgtgtttctgtttcccccaaagcccaaagacaccctgatgatctctcggacacctgaggtgacctgcgtggtggtggatgtgtctcacgaggaccccgaagtgaagttcaactggtacgtggacggcgtggaagtgcacaacgccaagaccaagccacgcgaagagcagtacaactctacctacagagtggtgtccgtgctcacagtgctgcaccaggactggctgaacggaaaagagtacaagtgcaaggtctctaacaaggctctgccagcccctatcgagaagaccatctccaaggccaagggccaacctagagagcctcaagtgtacaccctgcctcctagccgggacgagctgaccaagaaccaggtttccctgacctgtctggtgaaaggcttctacccctccgatatcgccgtggaatgggagtccaacggccagcctgagaacaactacaagaccacccctcctgtgctggactccgatggatccttcttcctgtatagcaagctgaccgtggacaagtccagatggcagcagggcaacgtgttcagctgttccgtgatgcacgaggccctgcataatcactacacccagaagtctctgagcctgtctcctggcaagtga

SEQ ID NO: 45 - DNA, 702 bp, synthetic construct
  source 1..702: mol_type = other DNA
atgggctggtcctgcatcatcctgttcctggtggctaccgccaccggcgtgcactccgaaatcgtgctaactcaaagtccagcaactctcagcctgagccctggcgagcgggctaccctgtcctgcaaggcctctcaggacatcaactcctacctgtcttggttccagcagaaaccaggccaggctcctagaaccctgatctacagagccaaccggctggtggacggcatccctgccagattctccggctctggctccggacaagattttacactgaccatctcctctctggaacctgaggacttcgccgtgtactattgtctgcagtacgacgagttccccttcaccttcggcggaggcaccaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 46 - DNA, 1410 bp, synthetic construct
  source 1..1410: mol_type = other DNA
atgggctggtcctgcatcatcctgttcctggtggctaccgccaccggcgtgcactcccaagtgcaactactagaaagtggtggtggtctcgtcaaacccggtggctccctgcggctgagctgtgctacctctggctacgtgttctccagatcctggatgaactggatccgccaggctcctggccagggcctggaatggatcggctggatctaccctggagatggcgataccaactacaacggcaaggtgaaggaccggtttacactgtctgccgacaaggccaagaactccgtgtacctgcagatgaactccctgagagccgaggacaccgctgtgtacttctgcgccagaggcggatatgacggctctccttggctgtcctactggggccaaggcaccctggtgaccgtgtccagcgcaagtactaagggtccaagtgtgtttccactggctccctctagcaagtccacctctggcggcaccgccgccctgggctgcctggtcaaggactacttccccgagcccgtcaccgtgtcctggaactccggcgctctgacctccggcgtgcataccttccctgctgtgctgcagtcttctggcctgtactccctgtcctccgtcgtgacagtgccttctagctctctcggcacacagacatacatctgcaatgtgaaccacaagccttccaacaccaaagtggataagaaggtggaacctaagtcctgcgacaaaacccacacctgtcctccttgccctgctcctgaagccgctggcggaccttctgtgtttctgtttcccccaaagcccaaagacaccctgatgatctctcggacacctgaggtgacctgcgtggtggtggatgtgtctcacgaggaccccgaagtgaagttcaactggtacgtggacggcgtggaagtgcacaacgccaagaccaagccacgcgaagagcagtacaactctacctacagagtggtgtccgtgctcacagtgctgcaccaggactggctgaacggaaaagagtacaagtgcaaggtctctaacaaggctctgccagcccctatcgagaagaccatctccaaggccaagggccaacctagagagcctcaagtgtacaccctgcctcctagccgggacgagctgaccaagaaccaggtttccctgacctgtctggtgaaaggcttctacccctccgatatcgccgtggaatgggagtccaacggccagcctgagaacaactacaagaccacccctcctgtgctggactccgatggatccttcttcctgtatagcaagctgaccgtggacaagtccagatggcagcagggcaacgtgttcagctgttccgtgatgcacgaggccctgcataatcactacacccagaagtctctgagcctgtctcctggcaagtga

SEQ ID NO: 47 - DNA, 702 bp, synthetic construct
  source 1..702: mol_type = other DNA
atgggctggtcctgcatcatcctgttcctggtggctaccgccaccggcgtgcactccgaaatcgtgctaactcaaagtccagattttcaatccgtgacccctaaagagaaggtcacaatcacctgtaaggcctctcaggacatcaactcctacctgtcctggttccagcagaagcctgaccagtcccctaagaccctgatctatagagccaaccggctggtggacggcgtgccaagcagattctccggctctggatctggccaggattttacactgaccatcaacagcctggaagccgaggatgctgctacctactactgcctgcagtacgacgagttccccttcaccttcggcggcggcaccaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 48 - protein, 5 aa, synthetic construct
TTIYY

SEQ ID NO: 49 - protein, 4 aa, synthetic construct
KTEL

SEQ ID NO: 50 - DNA, 20 bp, synthetic construct
  source 1..20: mol_type = other DNA
gggtcagaaggattcctatg

SEQ ID NO: 51 - DNA, 20 bp, synthetic construct
  source 1..20: mol_type = other DNA
ggtctcaaacatgatctggg

SEQ ID NO: 52 - DNA, 19 bp, synthetic construct
  source 1..19: mol_type = other DNA
cgaagccgggaagagcaag

SEQ ID NO: 53 - DNA, 23 bp, synthetic construct
  source 1..23: mol_type = other DNA
cgttcccactgctgatttatctg

SEQ ID NO: 54 - DNA, 18 bp, synthetic construct
  source 1..18: mol_type = other DNA
cctgacgggcaggtgatc

SEQ ID NO: 55 - DNA, 25 bp, synthetic construct
  source 1..25: mol_type = other DNA
atgaaagatggctggaagagagtct

SEQ ID NO: 56 - DNA, 24 bp, synthetic construct
  source 1..24: mol_type = other DNA
gagtactggatcgaccctaaccaa

SEQ ID NO: 57 - DNA, 23 bp, synthetic construct
  source 1..23: mol_type = other DNA
acacaggtctgacctgtctccat

SEQ ID NO: 58 - DNA, 21 bp, synthetic construct
  source 1..21: mol_type = other DNA
cccgaagcggactactatgct

SEQ ID NO: 59 - DNA, 24 bp, synthetic construct
  source 1..24: mol_type = other DNA
gttttctcatagatggcgttgttg